(19) United States
(12) Patent Application Publication
(10) Pub. No.: US 2026/0191766 A1
(43) Pub. Date: Jul. 9, 2026

(54) USE OF CONDITIONED MEDIUM DERIVED FROM CULTIVATION OF MESENCHYMAL STEM CELLS OF THE UMBILICAL CORD FOR INDUCING, STIMULATING AND PROMOTING HAIR GROWTH AND REGENERATION

(71) Applicant: CELLRESEARCH CORPORATION PTE. LTD., Singapore (SG)

(72) Inventors: Toan Thang PHAN, Singapore (SG); Nicholas LANG, Singapore (SG)

(73) Assignee: CELLRESEARCH CORPORATION PTE. LTD., Singapore (SG)

(21) Appl. No.: 19/130,893

(22) PCT Filed: Nov. 17, 2023
(86) PCT No.: PCT/SG2023/050772

§ 371 Date: May 16, 2025

(30) Foreign Application Priority Data
Nov. 17, 2022 (WO) PCT/SG2022/050834

(51) Int. Cl. A61K 8/98; A61Q 7/00; C12N 5/0775

(52) U.S. Cl. CPC A61K 8/982; A61Q 7/00; C12N 5/0668; C12N 2500/40; C12N 2501/11; C12N 2501/33; C12N 2501/39; C12N 2501/395

(57) ABSTRACT

The present invention relates to a method of inducing, stimulating and/or promoting hair growth and/or hair regeneration, wherein the method comprises treating the hair of a subject with conditioned medium derived from cultivation of mesenchymal stem cells of the umbilical cord. The present invention also relates to a method of alleviating and/or reducing hair loss and/or hair thinning, wherein the method comprises treating the hair a subject with conditioned medium derived from cultivation of mesenchymal stem cells of the umbilical cord. The invention also relates to a method of producing a conditioned medium, the method comprising: a) cultivating mesenchymal stem cells of the umbilical cord in a culture medium comprising DMEM (Dulbecco's modified eagle medium), F12 (Ham's F12 Medium), M171 (Medium 171) and FBS (Fetal Bovine Serum); b) removing the mesenchymal stem cells of the umbilical cord from the culture medium; wherein the conditioned medium is obtained by collecting the cell culture medium. The invention also relates to a conditioned medium obtained or obtainable by the producing method, a composition thereof and uses thereof.

### CROSS-REFERENCE TO RELATED APPLICATIONS

The present application claims the benefit of priority of International Patent Application No. PCT/SG2022/050834, filed 17 Nov. 2022 the content of which is hereby incorporated by reference it its entirety for all purposes.

### FIELD OF THE INVENTION

The invention relates to a method of inducing, stimulating and/or promoting hair growth and/or hair regeneration, wherein the method comprises treating the hair of a subject with conditioned medium derived from cultivation of mesenchymal stem cells of the umbilical cord. Equally, the invention relates to a method of alleviating and/or reducing hair loss and/or hair thinning, wherein the method comprises treating the hair of a subject with conditioned medium derived from cultivation of mesenchymal stem cells of the umbilical cord. These methods can be non-therapeutic and cosmetic methods and can be used to treat both male and female hair loss and/or thinning.

The present invention is also directed to conditioned medium, uses thereof, compositions comprising the same, and methods of producing the same, wherein the conditioned medium is derived from cultivation of mesenchymal stem cells of the umbilical cord. Particularly, the method of producing a conditioned medium derived from mesenchymal stem cells of the umbilical cord, the method comprises a) cultivating mesenchymal stem cells of the umbilical cord in a culture medium comprising DMEM (Dulbecco's modified eagle medium), F12 (Ham's F12 Medium), M171 (Medium 171) and FBS (Fetal Bovine Serum); (b) removing the mesenchymal stem cells of the umbilical cord from the culture medium; wherein the conditioned medium is obtained by collecting the cell culture medium. Subsequent to step b) of the method of producing a conditioned medium, the method may comprise one or more further steps of cultivating the mesenchymal stem cells of the umbilical cord in a further culture medium, optionally wherein the second (further) cell culture medium comprises a water-soluble antioxidant. The second cell culture medium may not comprise a growth factor and/or serum. In addition, the cells may be cultivated in this second cell culture medium at a concentration of about 1 million cells per 1 ml. The cultivation of the mesenchymal stem cells of the umbilical cord of step c) may be performed in the same culture medium as in step a), i.e., a culture medium comprising DMEM (Dulbecco's modified eagle medium), F12 (Ham's F12 Medium), M171 (Medium 171) and FBS (Fetal Bovine Serum or in a different cell culture medium as in step (a). The present invention also refers to conditioned medium, uses thereof and a composition comprising the conditioned medium which is derived from cultivation of mesenchymal stem cells of the umbilical cord and obtained or obtainable by the producing method of the present invention.

The invention also refers to uses of the conditioned medium and a composition thereof for inducing, stimulating and/or promoting hair growth and/or hair regeneration. The invention also refers to uses of the conditioned medium and a composition thereof for alleviating and/or reducing hair loss and/or hair thinning of a subject.

### BACKGROUND OF THE INVENTION

Hair consists of a hair shaft and a hair root. The hair shaft is the visible part on the surface of the skin and the hair root lies below the surface of the skin. At the base of the hair follicle is the dermal papilla, a pear shaped structure formed by a group of fibroblast cells, which supplies the hair root with blood, carries nutrients to produce new hair and controls the regeneration of the hair follicle throughout its lifecycle.

Normal hair follicles undergo a regenerative cycle defined by a growth stage (anagen), a degenerative stage (catagen), a resting stage (telogen), and a shedding stage (exogen). Anagen is the phase of hair follicle growth and involves regrowth of the cycling part of the hair follicle. In the anagen growth phase, dermal papilla fibroblasts secrete numerous growth factors that maintain active proliferation and differentiation of keratinocytes of the proximal hair bulb that forms the hair fiber. At the end of the growth phase, the dermal papilla can be separated from the hair follicle so that the supply of blood is cut off and the hair fiber can be pulled out, leading to hair loss.

According to the American Hair Loss Association, two thirds of mean above the ages of 35 show signs of hair loss. Although more prominent in men, hair loss is a condition that affects women generally from their 50s and 60s. Thus, recently, not only male-type but also hair-loss in females, and young adults are gradually on the increase. For example, hair-loss can be caused by various reasons including, for example, intrinsic factors such as genetic traits and mental stress in daily life. In this context, with the progress of industrial development, environmental pollution, stress, and aging, the symptoms of hair-loss symptoms become more serious, and with the advent of well-being era, there are growing concerns on the quality of life and physical appearance. Further, hair loss can also be linked to other factors such as illness, hormonal changes or side effects of medication. They may influence and inhibit hair production by aberrant hair follicle cycling and changes in the hair follicle morphology, leading to the physical destruction of the hair follicle.

For example, Alopecia areata is considered an organ-specific autoimmune disease stemming from loss of the hair follicle's (HF) immune privilege, so therapies are mostly immunosuppressive. As another example, Androgenic Alopecia (AGA) also known as male and female pattern baldness is the most common cause of hair loss in the world today. It can affect up to 50% of Caucasian males. AGA has a number of causes and is widely agreed to be multi-factorial with hereditary, environmental, dietary & hormonal influences. Effectively, hair follicle growth cycles reduce in time and the hair produced with in these cycles becomes shorter and thinner. Over time, the follicle becomes completely inactive within the scalp. Moreover, hair loss can be induced after viral infections such as a COVID-19 infection or a dengue virus infection as recently reported, e.g. Hussain N, Agarwala P, Iqbal K, Omar HMS, et al. A systematic review of acute telogen effluvium, a harrowing post-COVID-19 manifestation. J Med Virol. 2022 April; 94 (4): 1391-1401. doi: 10.1002/jmv.27534 or Chu & Yang, Dengue-associated telogen effluvium: A report of 14 patients. Dermatologica Sinica, Volume 35, Issue 3, September 2017, Pages 124-126.

In order to remedy the hair-loss phenomena described above, many types of hair-growth agents have been available on the market. Drug therapies specifically approved by the Food and Drug Administration (FDA) are limited to minoxidil and finasteride both of which may however present numerous side effects. Moreover, while these two compounds have effects regarding the prevention of hair-loss, their effects on the growth of hairs is negligible. Thus, there is a need to develop a cost-effective and safe technology for prevention of hair-loss which not only prevents hair loss but is also capable of effectively promoting hair-growth and regeneration.

Thus, there is also still a need for finding new compounds and methods of promoting hair growth and regeneration. Accordingly, it is an object of the invention to provide such compounds and methods.

### SUMMARY OF THE INVENTION

This object is accomplished by the methods, the conditioned medium and uses thereof having the features of the independent claims.

In a first aspect, the present invention provides a method of inducing, stimulating and/or promoting hair growth and/or hair regeneration, wherein the method comprises treating the hair of a subject with conditioned medium derived from cultivation of mesenchymal stem cells of the umbilical cord. This method can be a non-therapeutic or a cosmetic method.

In a second aspect, the present invention provides a method of alleviating and/or reducing hair loss and/or hair thinning, wherein the method comprises treating the hair of a subject with conditioned medium derived from cultivation of mesenchymal stem cells of the umbilical cord. Also this method can be a non-therapeutic or a cosmetic method.

In a third aspect, the present invention provides a method of producing a conditioned medium, the method comprising (a) cultivating mesenchymal stem cells of the umbilical cord in a culture medium comprising DMEM (Dulbecco's modified eagle medium), F12 (Ham's F12 Medium), M171 (Medium 171) and FBS (Fetal Bovine Serum); (b) removing the mesenchymal stem cells of the umbilical cord from the culture medium; (c) optionally cultivating the mesenchymal stem cells of the umbilical cord in a further culture medium, wherein the further culture medium is preferably serum-free and wherein optionally the further cell culture medium comprises a water-soluble antioxidant, wherein the conditioned medium is obtained by collecting the cell culture medium. In step (a) of this method, the mesenchymal stem cells of the umbilical cord may be cultivated to reach a confluency of about 70%, of about 80%, of about 90%, of about 95%, or cultivated to reach even 100% confluency (full confluency). In the optional step (c) of this method the mesenchymal stem cells can be further cultivated for any suitable period of time. Such a suitable period of time may, for example, be a period of 1 day, 2 days, 3 days, 4 days, 5 days, 6 days or 7 days.

In a fourth aspect, the present invention provides a conditioned medium derived from mesenchymal stem cells of the umbilical cord obtained or obtainable by the method according of the present invention.

In a fifth aspect, the present invention provides a comprising the conditioned medium of the present invention.

In a sixth aspect, the present invention provides the use of conditioned medium derived from cultivation of mesenchymal stem cells of the umbilical cord for inducing, stimulating and/or promoting hair growth and/or hair regeneration; and/or for alleviating and/or reducing hair loss and/or hair thinning of a subject.

### BRIEF DESCRIPTION OF THE DRAWINGS

The invention will be better understood with reference to the detailed description when considered in conjunction with the non-limiting examples and the drawings, in which:

FIG. 1A and FIG. 1B show representative images of immunohistochemical staining of elastin in human dermal fibroblasts (HDF) after culture in DMEM/10% FCS (control) vs. Red Deer Cord Lining Mesenchymal Stem Cell Conditioned Medium (RD-CLMSC-CM) for 48 hours. To compare the effects of DMEM/10% FCS and RD-CLMSC-CM on the expression of elastin, HDF (n=14) with varying donor profiles (subject age from 23 to 73 years, skin from various sites: forehead, eyelid, cheek, neck, temple) were cultured in DMEM/10% FCS (control) and RD-CLMSC-CM for 48 hours before immunocytochemical staining for elastin. Optical density was measured using ImageJ. RD-CLMSC-CM upregulated HDF expression of elastin by 56% compared to HDF cultured in DMEM/10% FCS (control).

FIG. 2A and FIG. 2B show representative images of immunohistochemical staining of hyaluronic acid (HA) in HDF after culture in DMEM/10% FCS (control) vs. RD-CLMSC-CM for 48 hours. To compare the effects of DMEM/10% FCS and RD-CLMSC-CM on the expression of HA, HDF (n=14) with varying donor profiles (subject age from 23 to 73 years, skin from various sites: forehead, eyelid, cheek, neck, temple) were cultured in DMEM/10% FCS (control) and RD-CLMSC-CM for 48 hours before immunocytochemical staining for HA. Optical density was measured using ImageJ. RD-CLMSC-CM upregulated HDF expression of hyaluronic acid by 83% compared to HDF cultured in DMEM/10% FCS (control).

FIG. 3 depicts total cell counts that were performed to compare the proliferative effects of DMEM/10% FCS (Control), Human Foreskin Fibroblast conditioned medium (FSF-CM), Human Cord Lining Mesenchymal Stem Cell Conditioned Medium (H-CLMSC-CM) and Red Deer Cord Lining Mesenchymal Stem Cell Conditioned Medium (RD-CLMSC-CM) on aged skin HDF. Starting with the same seeding density and after 5 days incubation in test medium, average total cell counts were calculated for each group. In comparison to the control, the highest increase in average total cell counts was seen in the RD-CLMSC-CM (113%), followed by the H-CLMSC-CM group (112%), and then the FSF-CM group at (16%).

FIG. 4 shows in-vitro ‘scratch’ wound assay to compare the pro-migrative effects of Red Deer Cord Lining Mesenchymal Stem Cell Conditioned Medium (RD-CLMSC-CM) and Human Foreskin Fibroblast conditioned medium (FSF-CM). A ‘scratch’ wound was created on confluent human dermal fibroblast (HDF) monolayer with a p200 pipette tip and images taken on Day 0 and Day 5 to document the progress of migration. ‘Scratch’ wounds were completely filled by HDF cultured in RD-CLMSC-CM on Day 5 as compared to 80% in FSF-CM.

FIG. 5 shows superior views of scalp before (left) and after 6 weeks of treatments and 6 treatments later (right) with a composition comprising conditioned medium derived from the cultivation of mesenchymal stem cells of the umbilical cord.

FIG. 6A-G show superior view of scalp before and after treatment (FIG. 6B-G) or in a weekly (FIG. 6A, B) interval during treatment period with a composition comprising conditioned medium derived from the cultivation of mesenchymal stem cells of the amniotic membrane of the umbilical cord of red deer (Red Deer Cord Lining Mesenchymal Stem Cell Conditioned Medium).

FIG. 7 shows superior views of a scalp of a patient suffering from COVID-19 induced hair loss with scalp visibility, hair fragility, and poor hair density, wherein the upper photograph shows the sculp prior to treatment. The photograph in the middle shows a superior view of the patient's scalp 3 months posttreatment with less scalp visibility and commencement of regrowth of frontal hairline. The lower photograph shows results 6 months after commencement of five monthly stem cell serum applications with minimal scalp visibility and restoration of hair density.

FIG. 8 shows human follicle dermal papilla cells (HFDPC) before treatment, after treatment with a concentration of 2 μg/mL Minoxidil, 0.2 μg/mL Minoxidil, after treatment with conditioned medium at a 160fold dilution (legend “PC 160 times dilution) and at a 320fold dilution (legend “PC 320 times dilution”) compared to the negative control (NC) at a 160fold and a 320fold dilution (legend “NC 320 times dilution” and “PC 320 times dilution”).

FIG. 9 shows TNF-α release in Normal Human Epidermal Keratinocytes (NHEK) after exposure to UVB (UV+) and without exposure to UVB (UV−) as negative control. Inhibition of TNF-α production in the presence of conditioned medium diluted by a factor of 2, 4, 8, 16, 32 and 64 and in the presence of Vitamin D. FIG. 9 shows that in this experiment conditioned medium of the invention reduces the expression/production of TNF-α as a key inflammatory cytokine by 30×, comparable to Vitamin D.

FIG. 10 shows a technical information sheet of Lonza for Dulbecco's modified eagle medium, including the catalogue number of the DMEM used for the making of the illustrative example of a medium (PTT-6) in the Experimental Section.

FIG. 11 shows a technical information sheet of Lonza for Ham's F12 medium.

FIG. 12 shows a technical information sheet of Lonza for DMEM: F12 (1:1) medium, including the catalogue number of the DMEM: F12 (1:1) medium used for the making of the illustrative example of a medium (PTT-6) in the Experimental Section.

FIG. 13 shows a technical information sheet of Life Technologies Corporation for M171 medium, including the catalogue number of the M171 medium used for the making of the illustrative example of a medium (PTT-6) in the Experimental Section.

FIG. 14 shows a list of ingredients, including their commercial supplier and the catalogue number that have been used in the Experimental Section for making the medium PTT-6.

FIG. 15 shows the Norwood classification of patterned hair loss in males.

FIG. 16 shows the Ludwig classification of patterned hair loss in females.

FIG. 17 shows objective assessment (Hairmetrix®) of 10 patients who suffer from hair loss and that applied a composition containing condition medium of the invention on their capillitium, with FIG. 17A showing follicular units per square centimeter (FU per sqcm) over the treatment period of 12 weeks, FIG. 17B showing the intra-follicular distance (mm) over the treatment period of 12 weeks, FIG. 17C showing total hair count per square centimeter over the treatment period of 12 weeks and FIG. 17D showing the sum of hair width per square centimeter (in millions per sqcm) over the treatment period of 12 weeks.

FIG. 18 shows the patients' subjective assessment by standardized questionaires, with FIG. 18A showing the satisfaction of the patients with hair growth, FIG. 18B showing the likelihood of the patients for hiding hair and FIG. 18C showing the patients' quality of life affected by their hair loss.

FIG. 19 shows Table 6 with the results off an objective assessment (Hairmetrix®) of Capllitium by main outcome parameters (mean values with standard deviations (mean/SD)) and P values for linear regression model) over the treatment period of 12 weeks. The main outcome parameters shown in Table 6 are (from top to bottom) intrafollicular distance (mm), T: V ratio, folliucular units (FU) per sqcm, average hairs per FU, average hair width (micm), terminal hair count per sqcm, total hair count per sqcm (Total), sum of terminal hair width (micm) per sqcm and sum of total hair width (micm) per sqcm)

FIG. 20 shows Table 7 with results for the patients' subjective assessment by standardized questionnaires. Shown are main outcome parameters (mean values with standard deviations (mean, SD)) and P-values for linear regression model over the treatment period of 12 weeks. The main outcome parameters shown in Table 7 are (from top to bottom) satisfaction with hair growth, satisfaction with hair density, satisfaction with hair thickness, satisfaction with hail quality, satisfaction with result, comfortable with hair, confidence, friends/family addressing hair quality, thinking of how hair looks like, hiding hair, re-arranging hair and affection quality of life.

### DETAILED DESCRIPTION

The present invention is directed to methods that are all suitable for inducing, stimulating and/or promoting hair growth and/or hair regeneration by using conditioned medium derived from cultivation of mesenchymal stem cells of the amniotic membrane of the umbilical cord. Additionally, the methods of the present invention are all suitable for alleviating and/or reducing hair loss and/or hair thinning by using conditioned medium derived from cultivation of mesenchymal stem cells of the umbilical cord. As such, the present invention is based on the discovery of the fact that conditioned medium derived from the cultivation of mesenchymal stem cells of the umbilical cord secretes biological factors (e.g., proteins) which provide excellent hair-loss preventing and hair growth effects of the conditioned medium used for treating the hair, particularly the skin at the hair line, of a subject. In this regard, the inventors surprisingly identified that mesenchymal stem cells of the umbilical cord secrete biological factors effective on hair-growth, and also confirmed that conditioned medium obtained by cultivating mesenchymal stem cells of the umbilical cord exhibited highly effective hair-growth promoting effects.

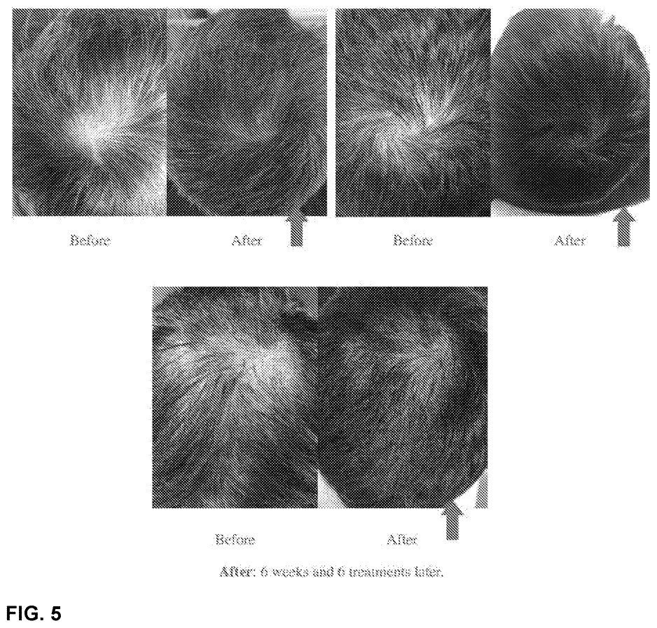

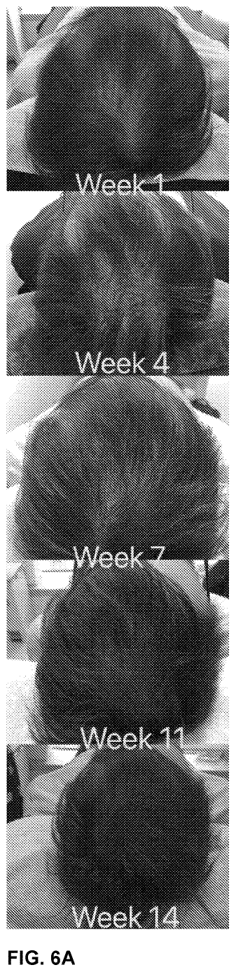

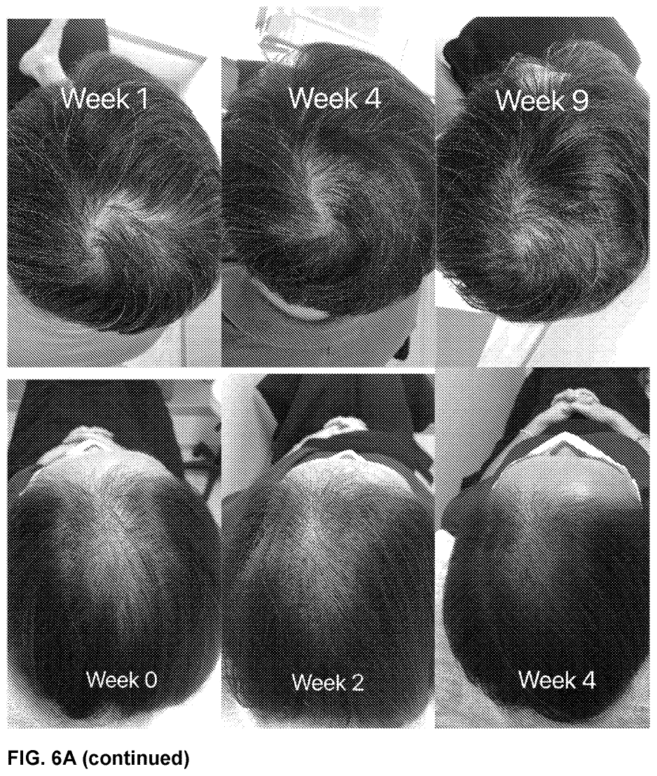

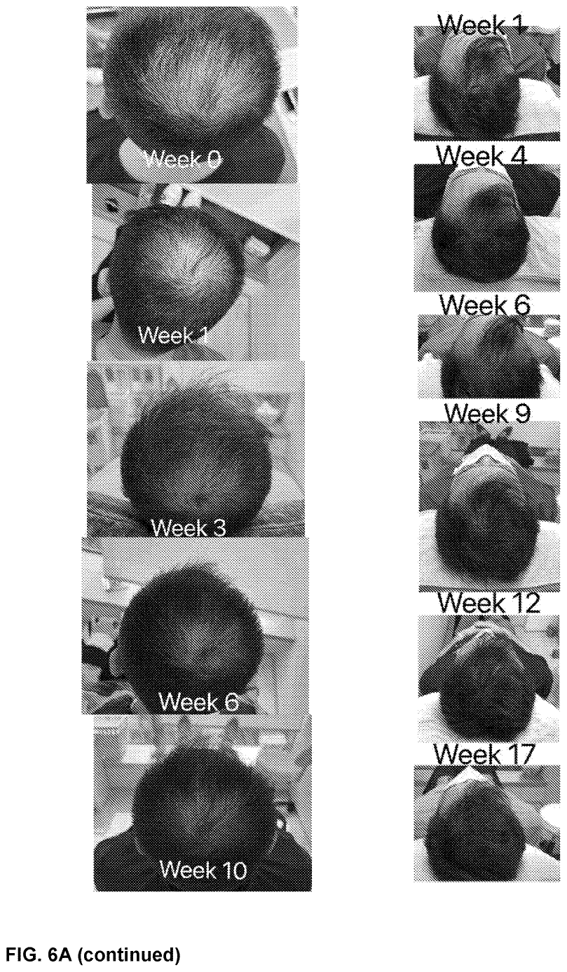

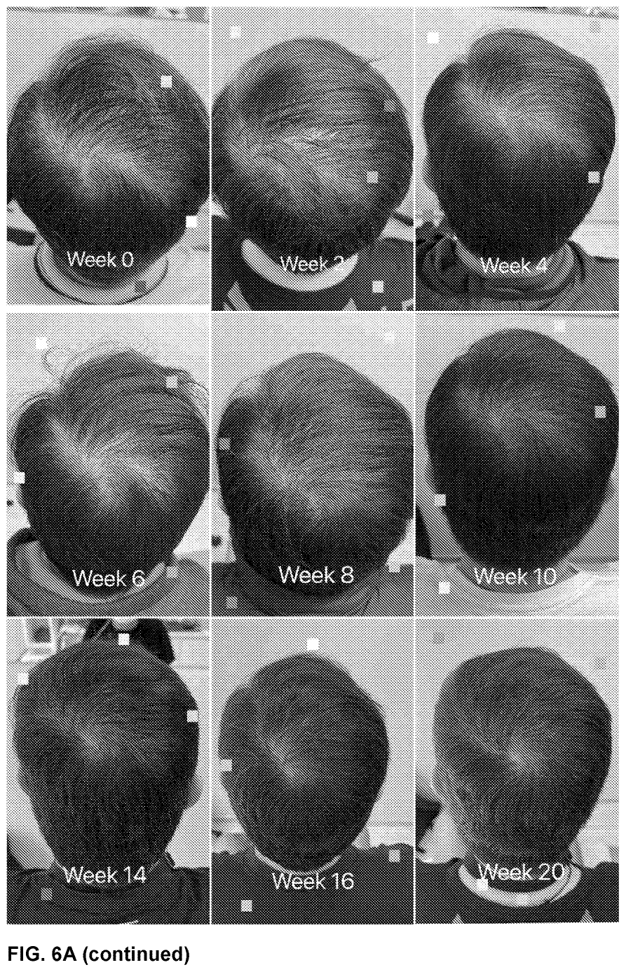

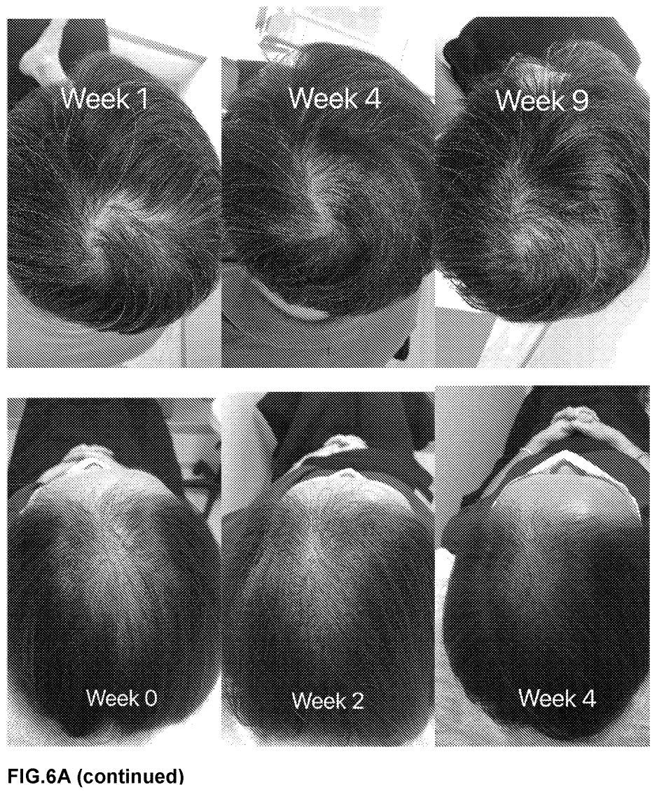

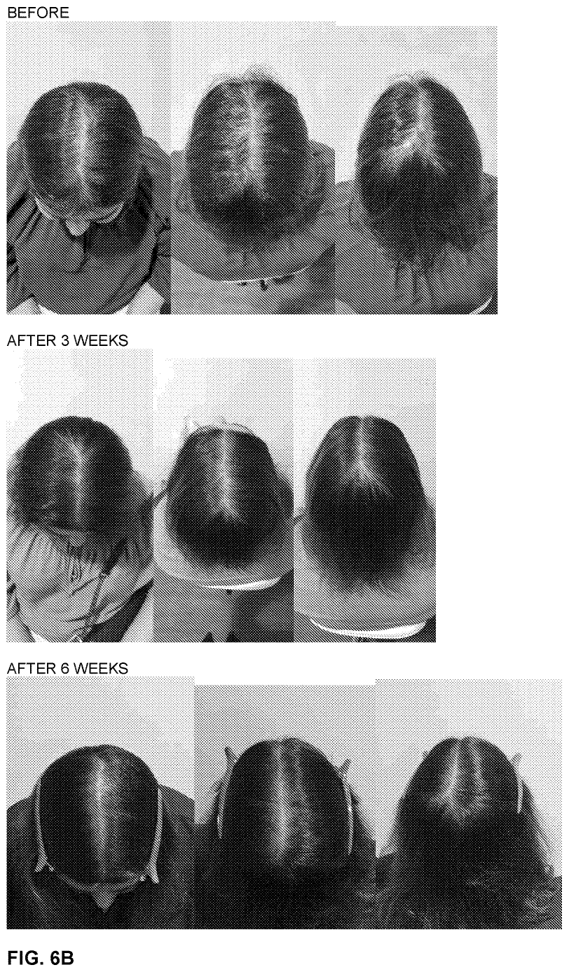

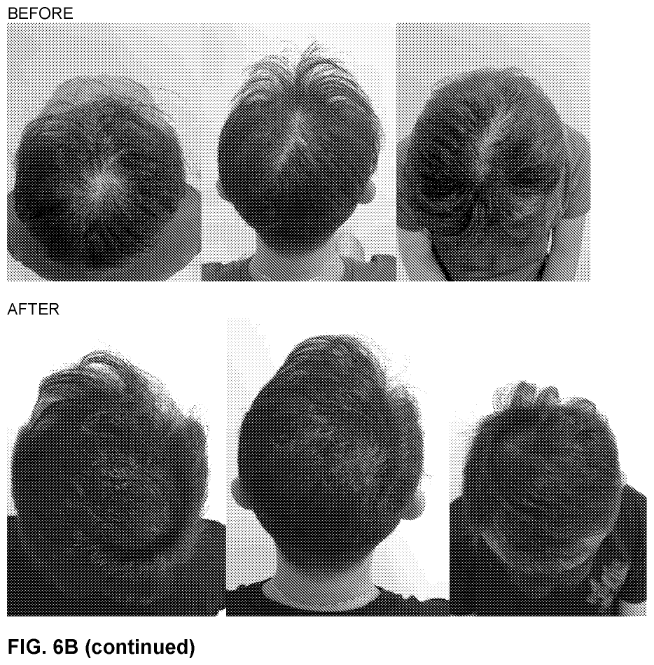

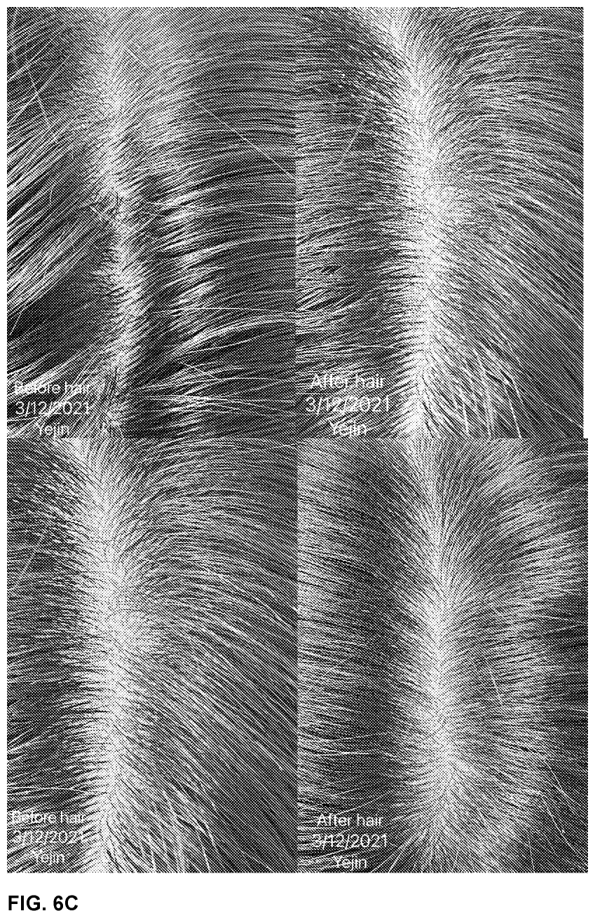

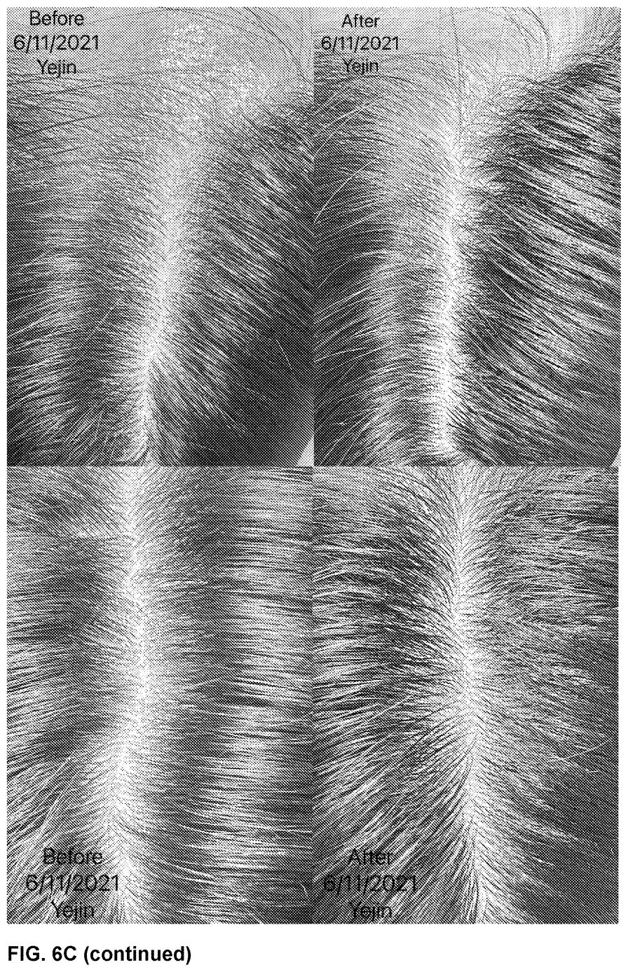

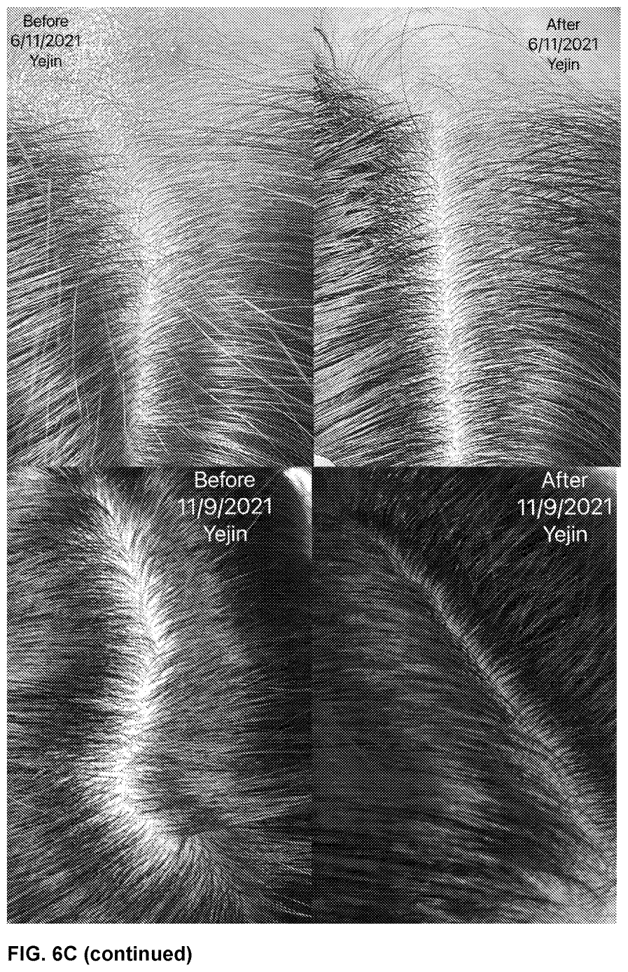

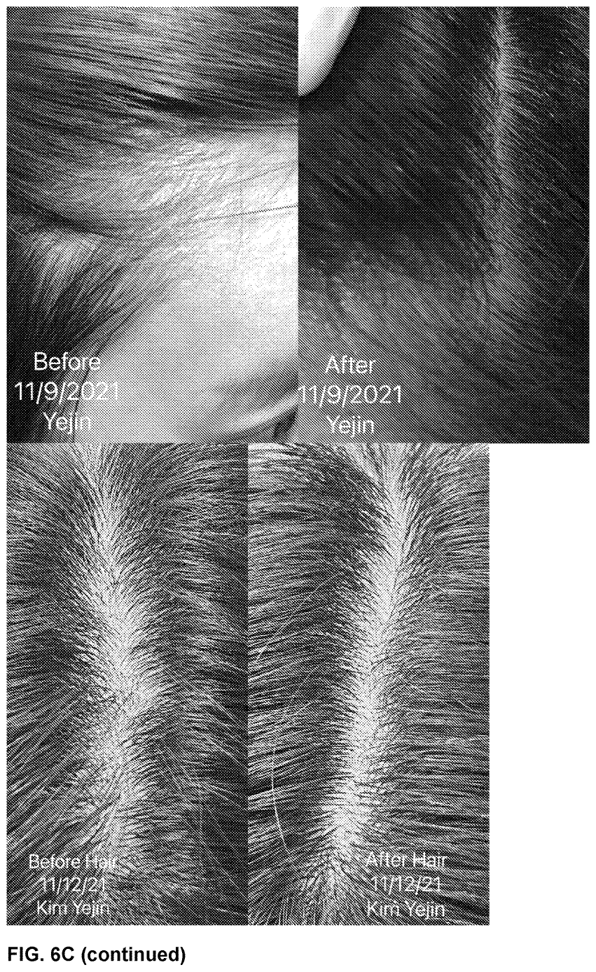

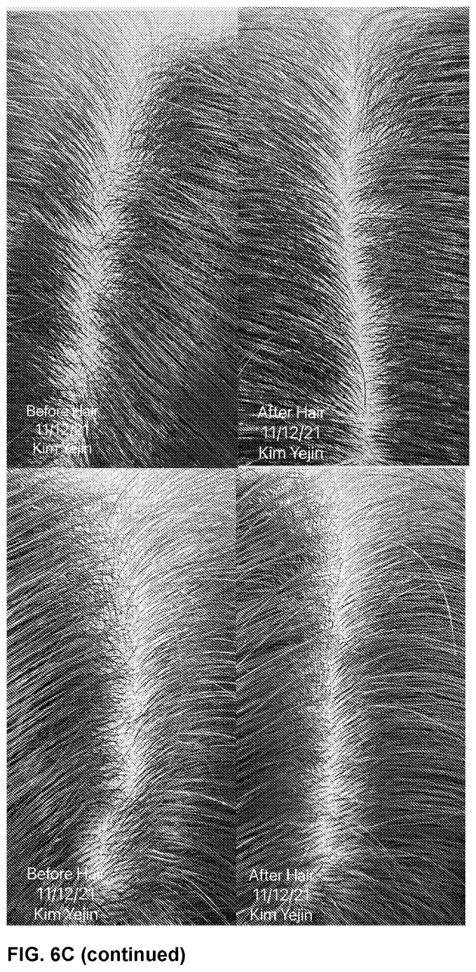

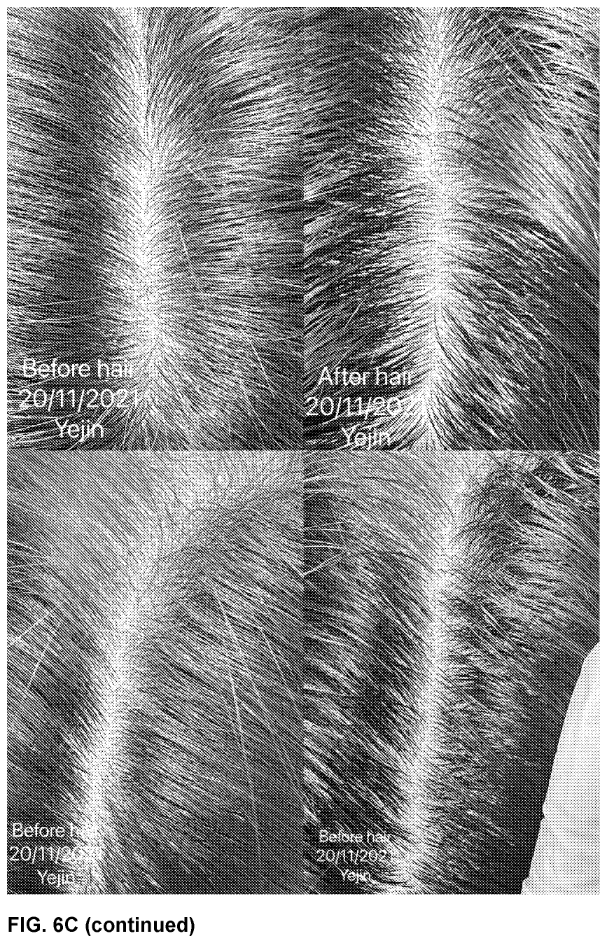

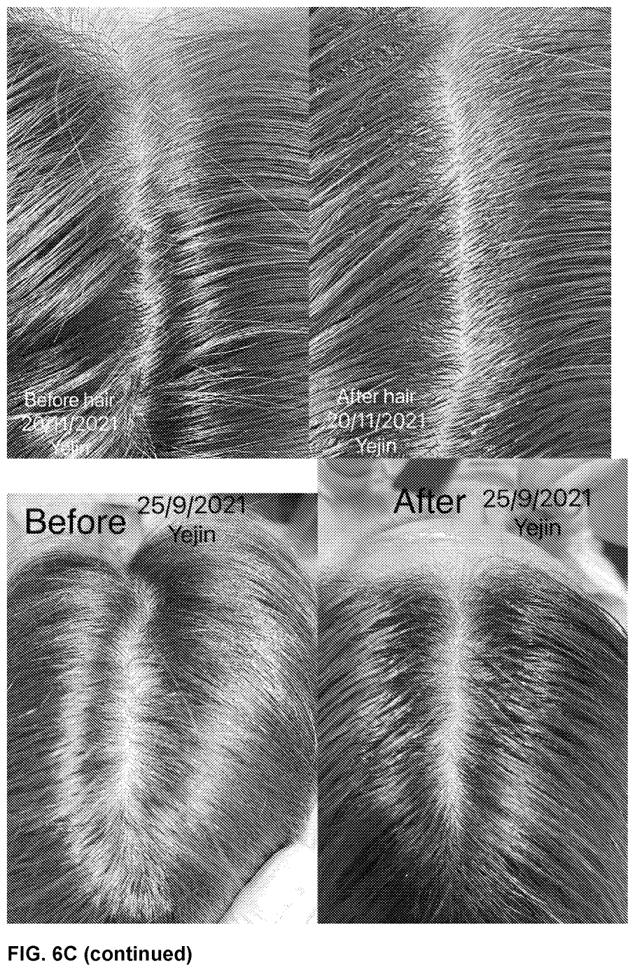

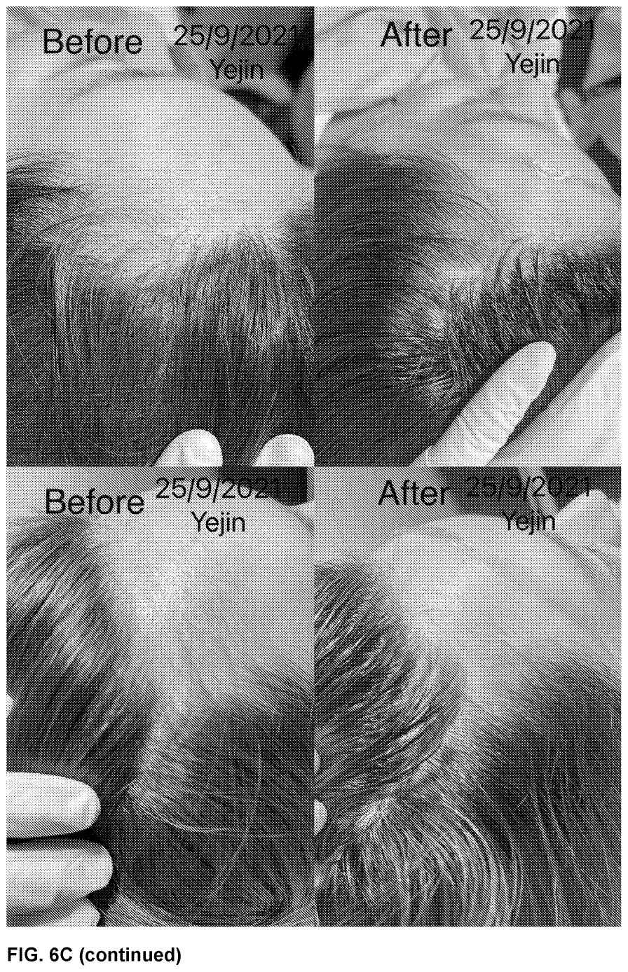

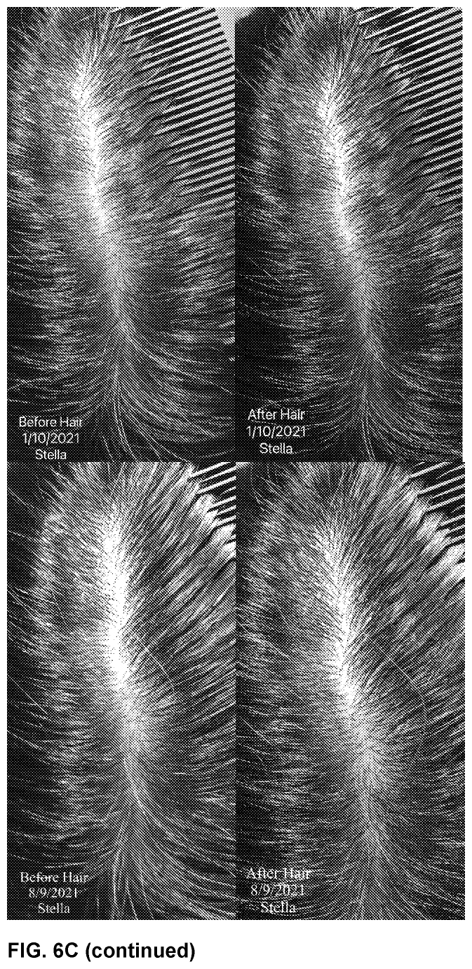

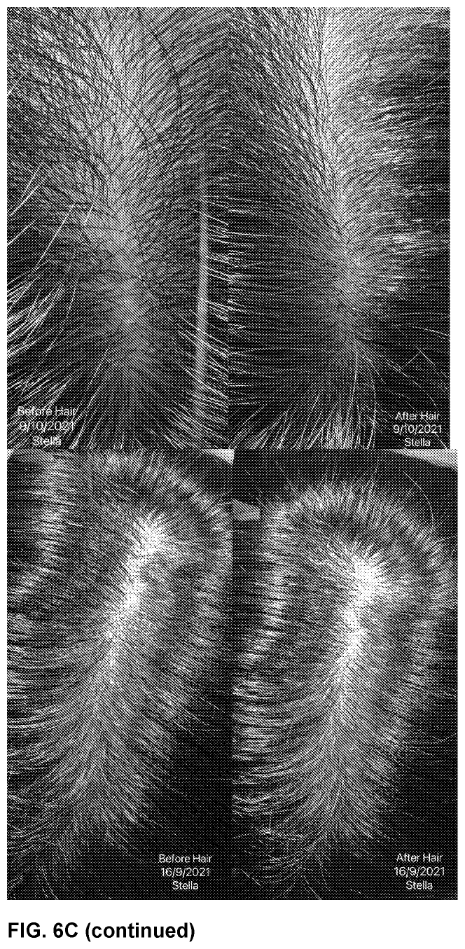

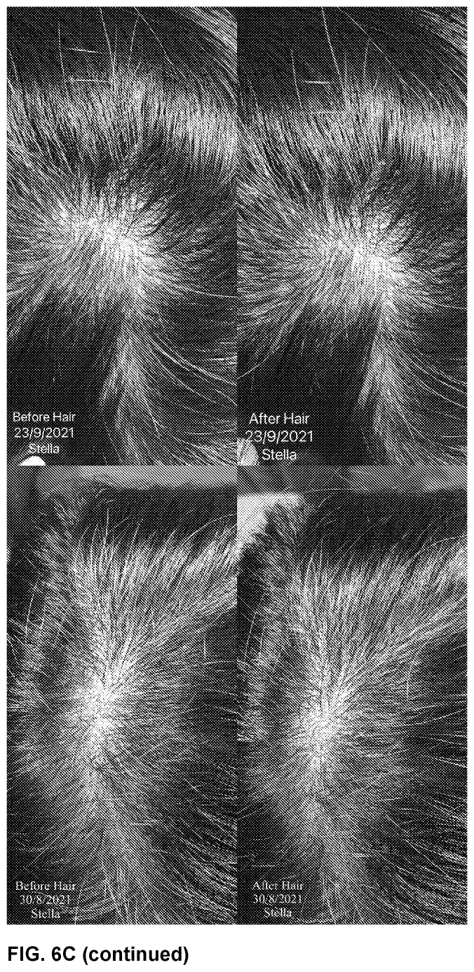

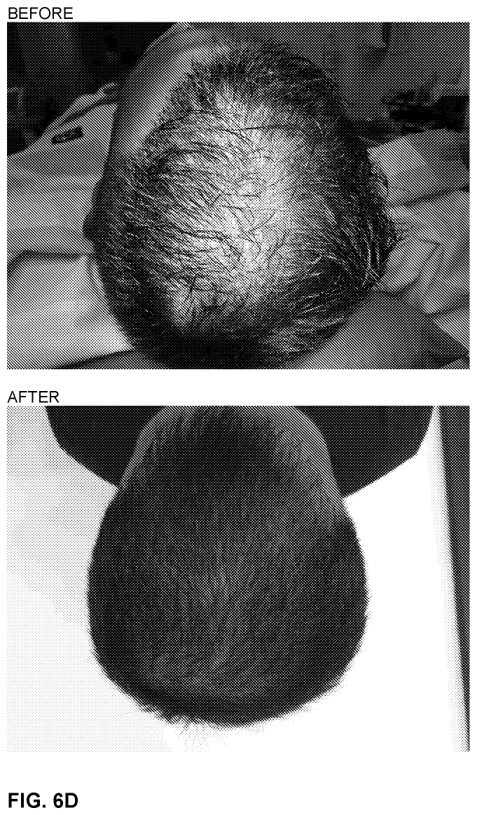

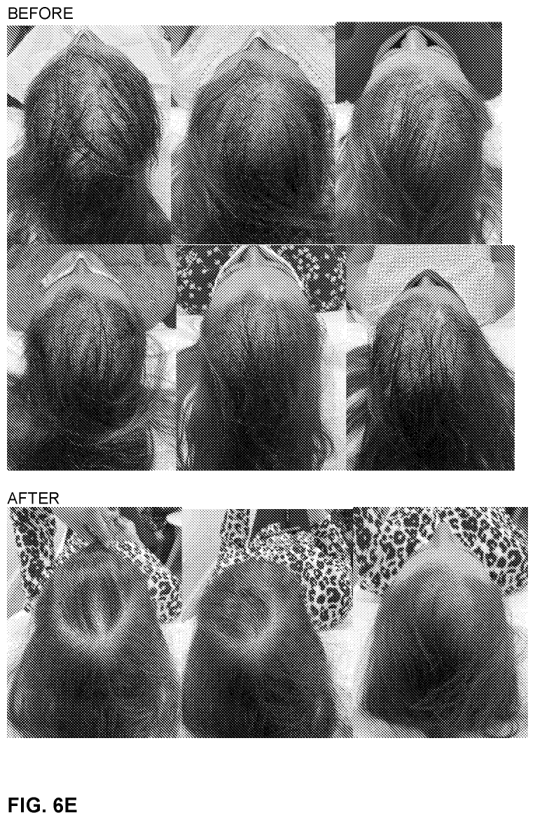

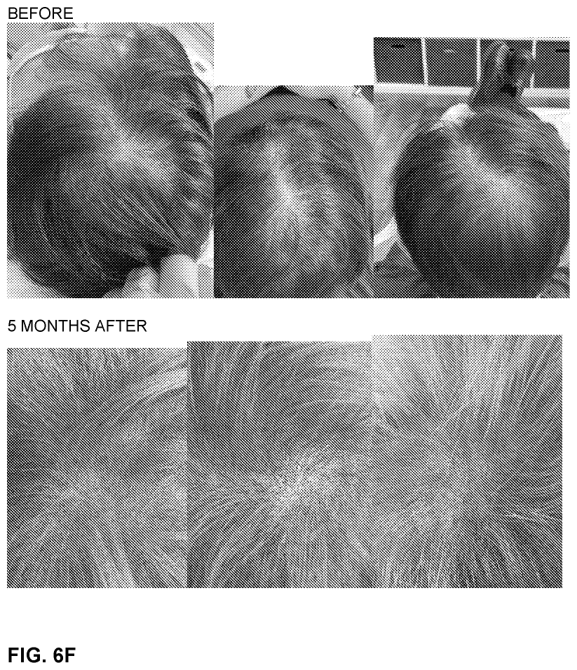

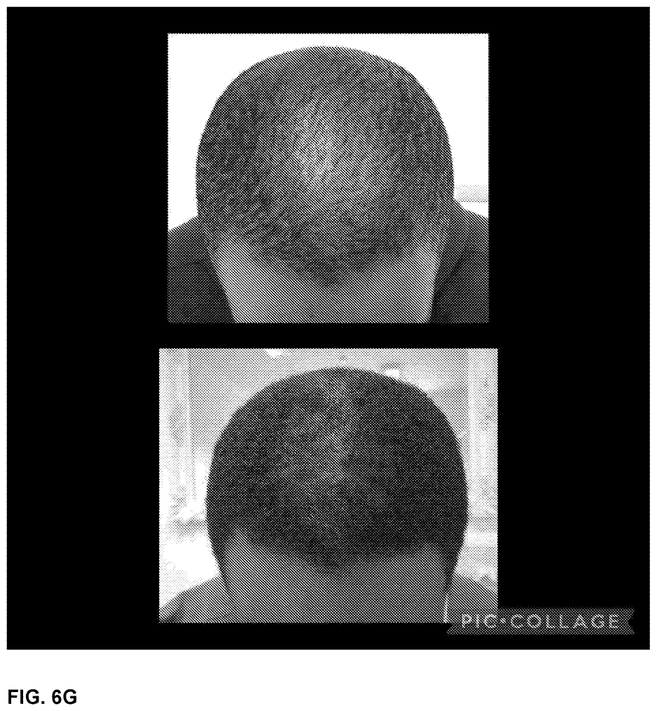

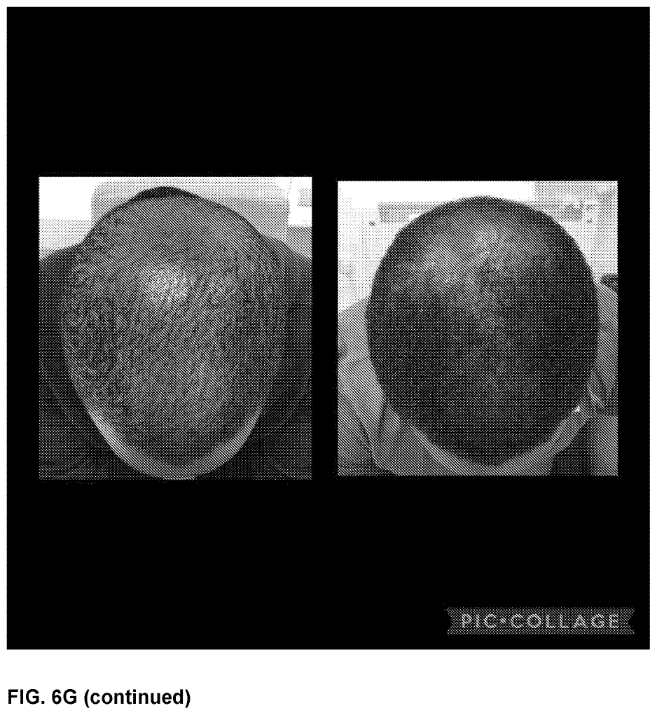

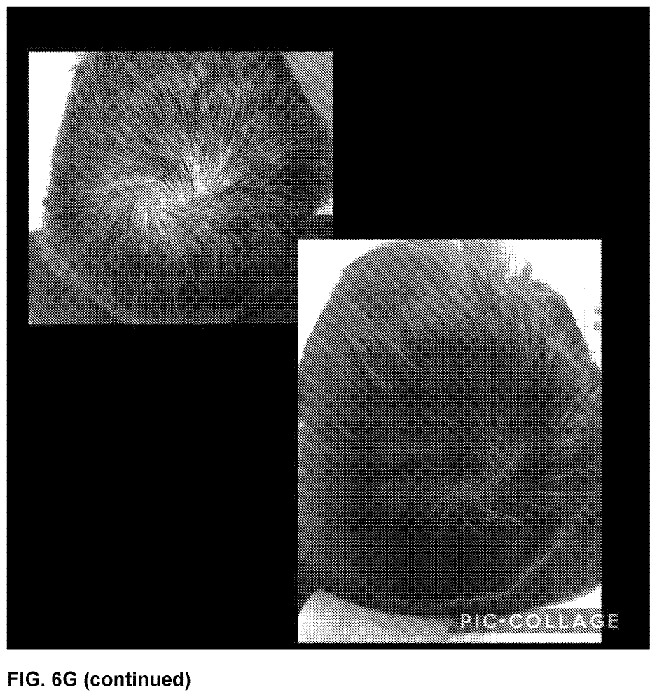

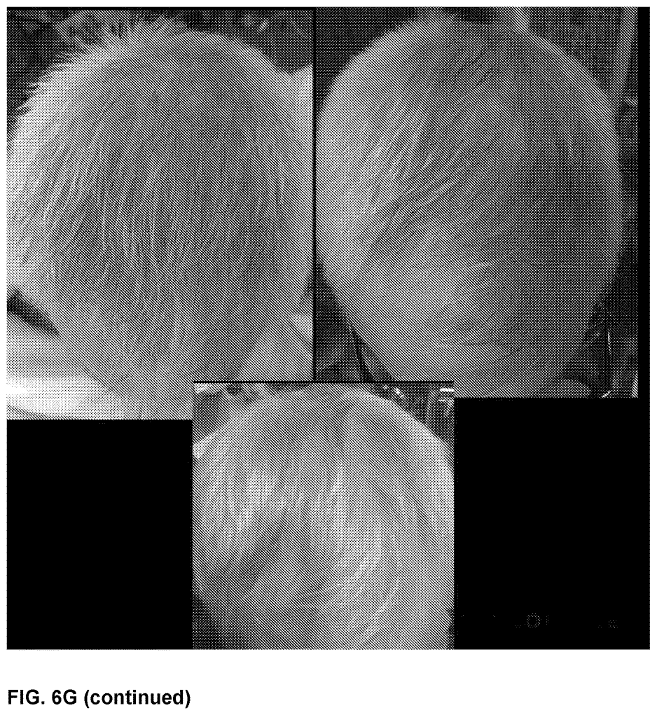

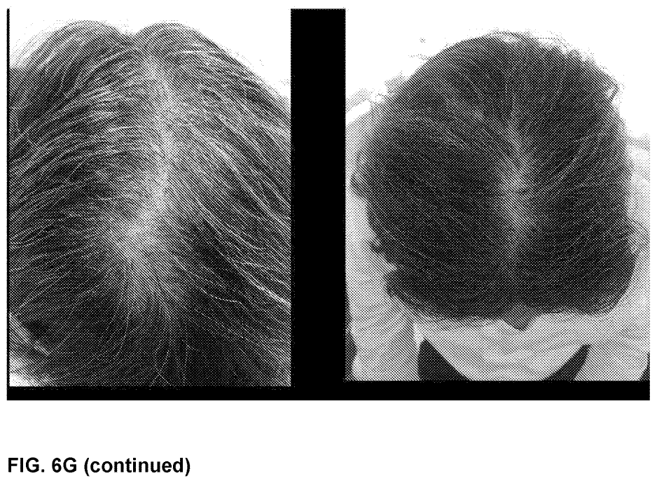

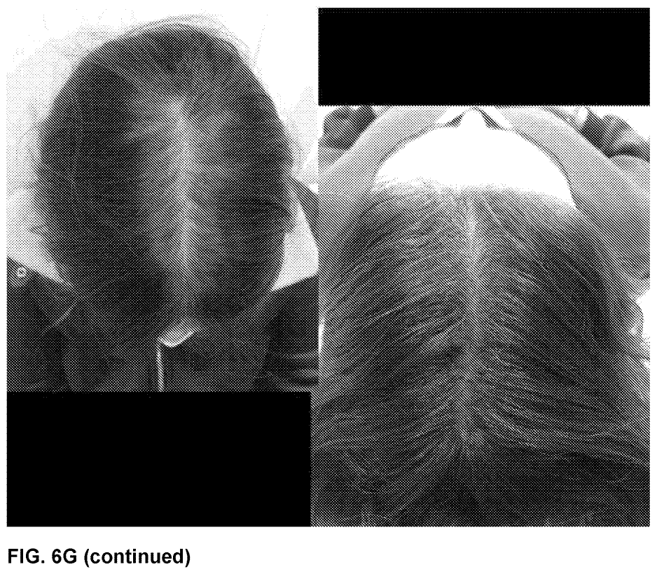

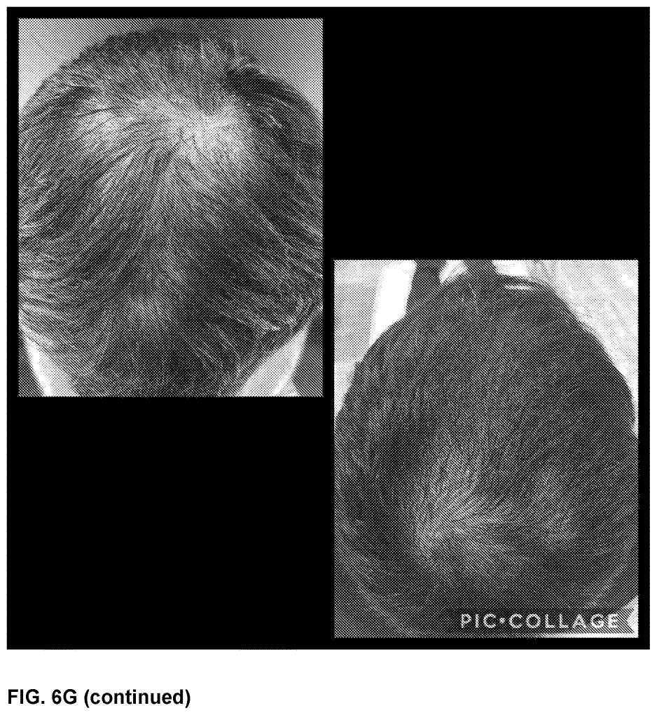

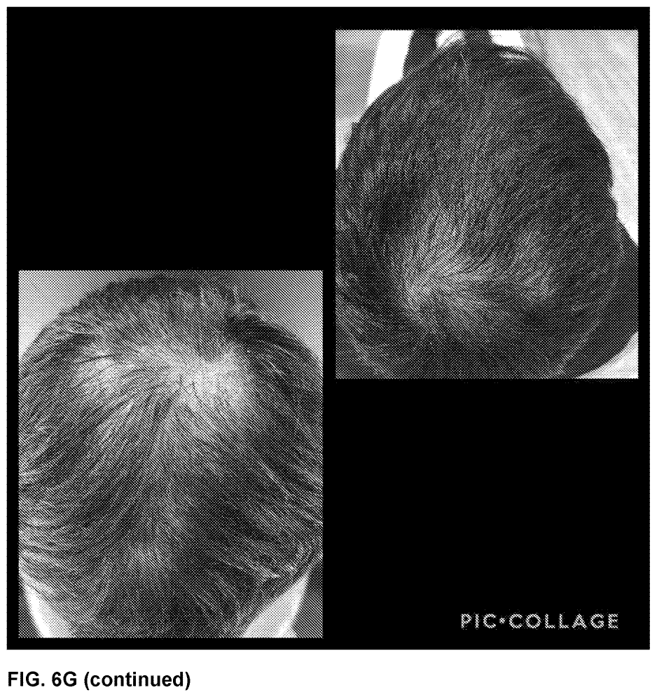

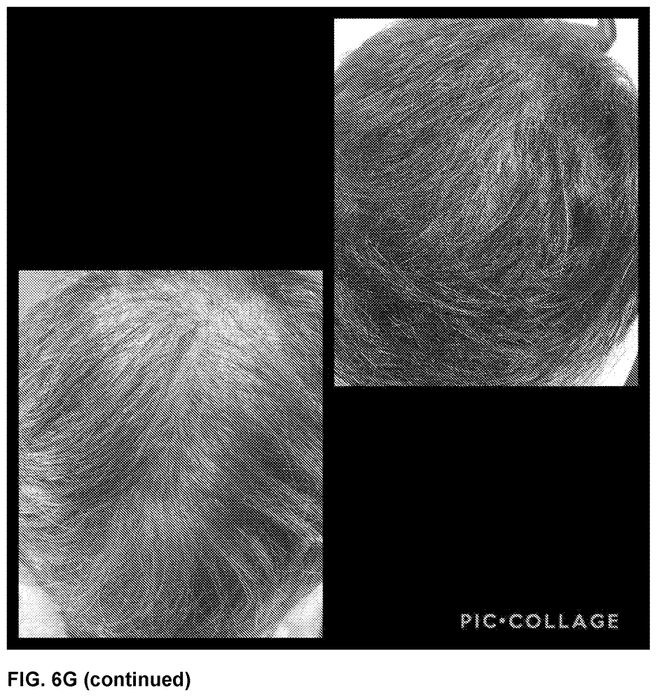

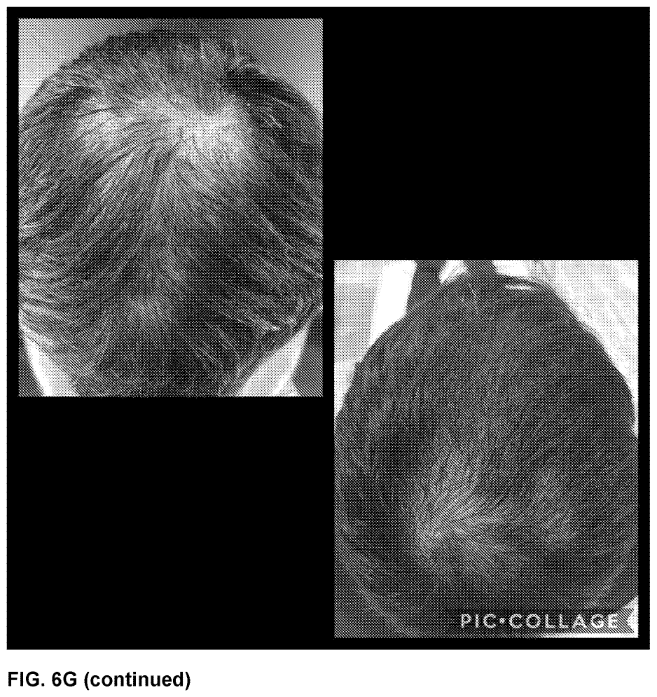

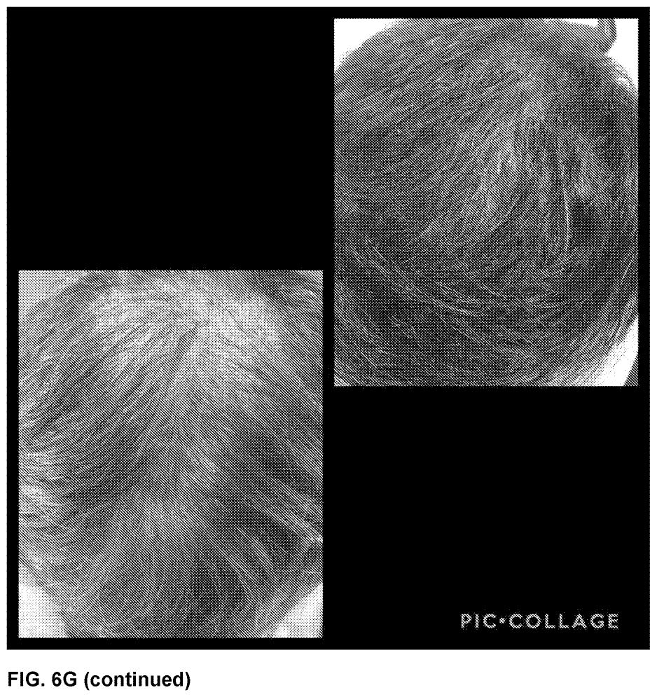

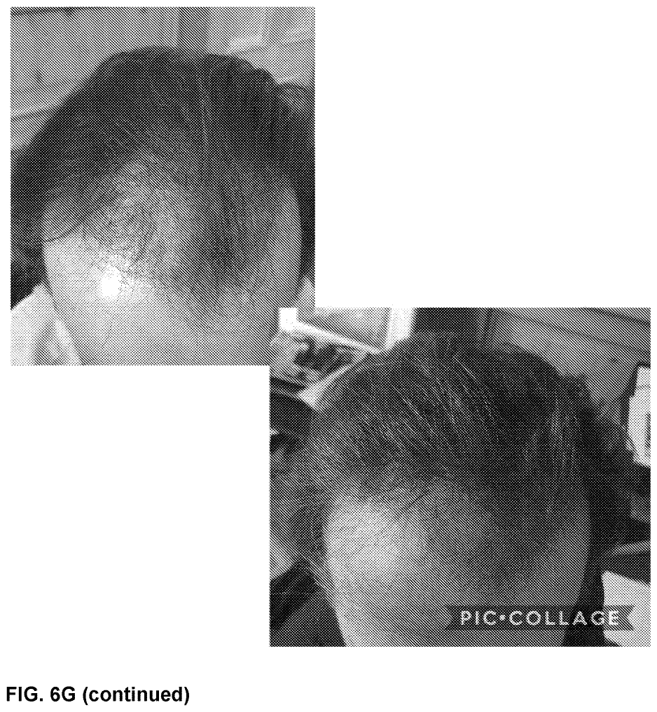

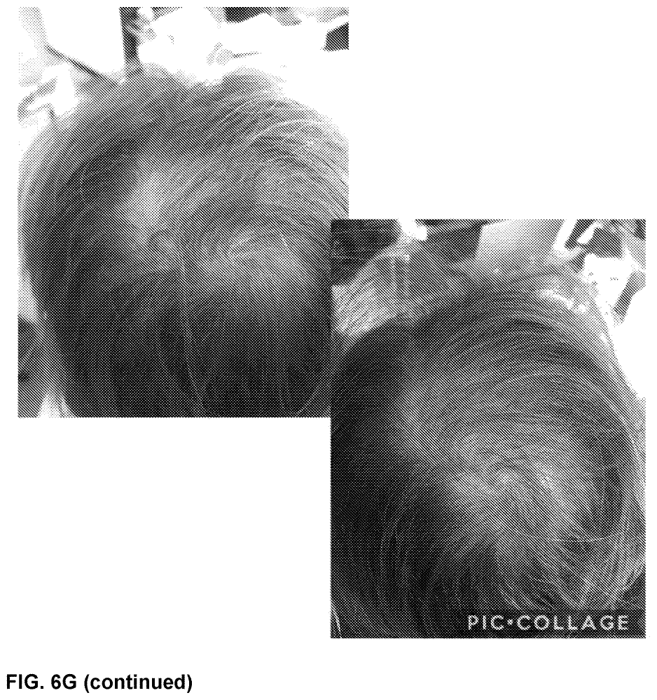

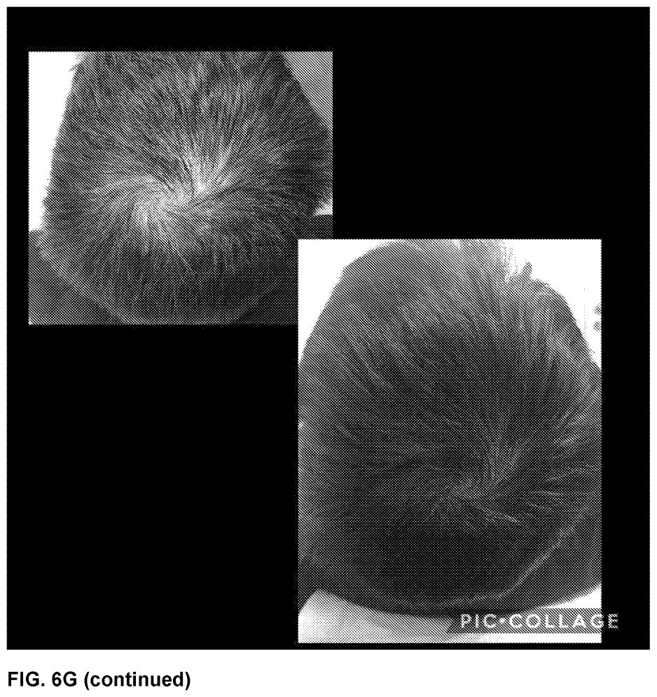

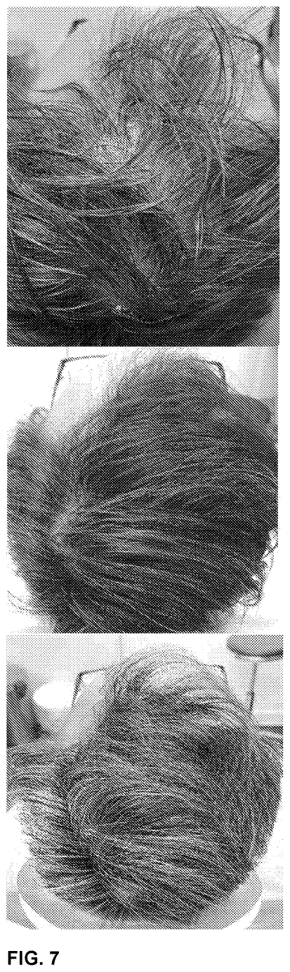

As shown in the experimental section, the present invention employs conditioned medium derived from cultivation of mesenchymal stem cells of the umbilical cord which is demonstrated to effectively and consistently improve hair quality, density and coverage from underactive or dormant hair follicles in patients suffering from hair thinning/hair loss. For example, as demonstrated in the experimental section, after 6 weeks of treatment with conditioned medium derived from the cultivation of mesenchymal stem cells of the umbilical cord (CALECIM®), patients with manifestation of hair thinning/hair loss had objective improvement in hair density and coverage (Example 6; FIGS. 6A-K). As another example, conditioned medium derived from the cultivation of mesenchymal stem cells of the umbilical cord (CALECIM®) stimulates hair growth in patients suffering from androgenic alopecia (AGA) (Example 7, FIG. 5)—which is also known as male and female pattern baldness and is the most common cause of hair loss in the world today. Moreover, a patient who developed telogen effluvium (TE) which refers to diffuse alopecia after a COVID-19 infection received five treatments of conditioned medium derived from the cultivation of mesenchymal stem cells of the umbilical cord (CALECIM®) monthly after not responding to conventional hair loss therapy and has progressive improvement in hair density with regrowth of frontal hairline and villus hair in the prefrontal region (Example 8, FIG. 7). In this context, the finding of the present invention is that conditioned medium derived from the cultivation of mesenchymal stem cells of the umbilical cord has excellent effects on hair growth.

Accordingly, the present invention relates to a medium which is conditioned by cultivation of mesenchymal stem cells of the umbilical cord. Thus, the conditioned medium is obtained by cultivation of mesenchymal stem cells of the umbilical cord in cell culture medium, as described herein, and separating the resulting medium from the cells, so that the conditioned medium of the present invention contains the secreted stem cell products (referred to as biological factors). Thus, the conditioned medium contains biological factors and substantially no or no stem. Biological factors that may be in the conditioned medium include but are not limited to growth factors, exosomes, hormones, cytokines, extracellular matrix, proteins, vesicles, antibodies, chemokines, receptors, inhibitor, and granules. Such conditioned medium, and combinations of any of the biological factors comprised therein, including but not limited to growth factors, may be used in the method of the present invention.

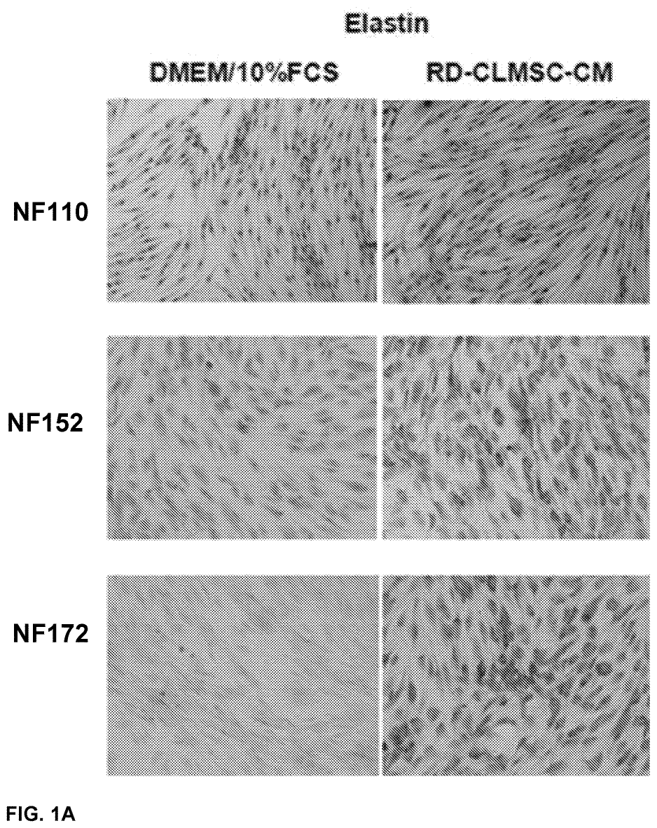

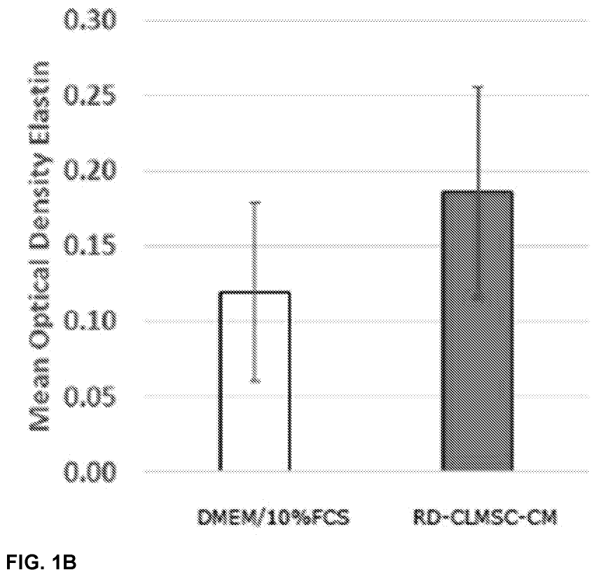

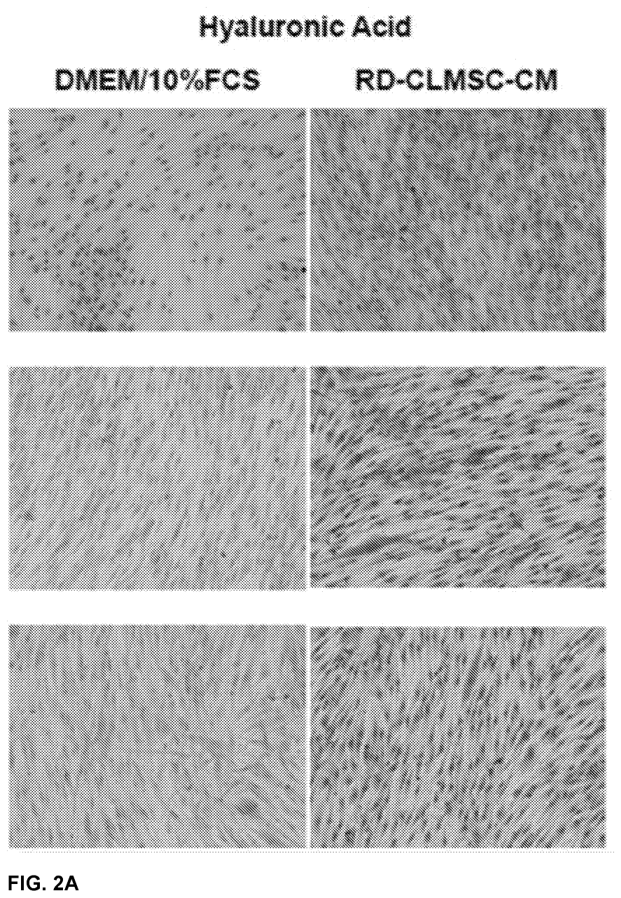

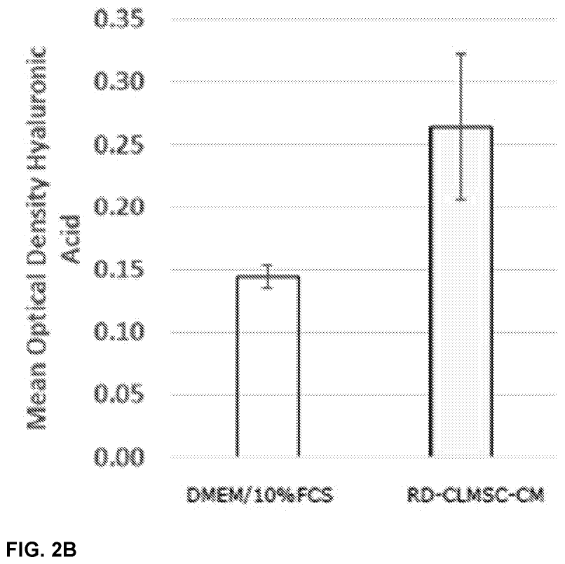

As shown here in the experimental examples, conditioned medium derived from cultivation of mesenchymal stem cells of the umbilical cord is particularly rich in growth factors, which are proposed to aid in reversing disrupted hair follicle activity in conditions of hair thinning/hair loss such as conventional alopecia and COVID-19 induced hair loss. For example, epidermal growth factor (EGF) is known to be positively implicated with hair growth since EGF promotes the anagen phase and controls the elongation of follicles and co-stimulates angiogenesis; see Mak KK, Kingston SY, Epidermal growth factor as a biologic switch in hair growth cycle. J. Invest Dermatol 2001: 117 (6): 1594-600). As another example, fibroblast growth factor 8FGF is known to be positively implicated with advancement of hair follicles. As shown in the experimental section herein (Example 2), conditioned medium-derived from cultivation of red deer mesenchymal stem cells of the umbilical cord (RD-CLMSC) in a medium comprising DMEM (Dulbecco's modified eagle medium), F12 (Ham's F12 Medium), M171 (Medium 171) and FBS (Fetal Bovine Serum)—when applied to human dermal fibroblasts (HDF) to assess the effect of conditioned medium on human skin, significantly upregulates the expression of elastin and hyaluronic acid compared to application of DMEM/10% FCS to HDS (Example 3, FIGS. 1 and 2). Without wishing to being bound by theory, the upregulation of elastin and hyaluronic acid expression present in RD-CLMSC-CM is due to presence of growth factors including transforming growth factor (TGF) beta1 and TGF beta2, insulin-like growth factor 1 (IGF-1) and platelet-derived growth factor (PDGF) and fibroblast growth factor 7 (FGF-7) present in RD-CLMSC-CM.

In this regard, the involvement of the presence of growth factors in inducing enhanced elastin and hyaluronic acid expression is known to the person skilled in the art. In humans, Transforming Growth FactorB1 (TGF-β1) plays a central role in the expression of tropoelastin (TE), the soluble form of elastin; see Kuang, P. P., et al., Activation of elastin transcription by transforming growth factor-beta in human lung fibroblasts. Am J Physiol Lung Cell Mol Physiol, 2007. 292 (4): p. L944-52. TGF-β1 also stabilizes tropoelastin mRNA transcripts; see Kähäri, V. M., et al., Transforming growth factor-beta up-regulates elastin gene expression in human skin fibroblasts. Evidence for post-transcriptional modulation. Lab Invest, 1992. 66 (5): p. 580-8; and Kucich, U., et al., Stabilization of elastin mRNA by TGF-beta: initial characterization of signaling pathway. Am J Respir Cell Mol Biol, 1997. 17 (1): p. 10-6. Interestingly, a combination of TGF-β1 and hyaluronic acid oligomers synergistically enhanced the levels of elastin in the extracellular matrix of cultured vascular smooth muscle cells; see Joddar, B. and A. Ramamurthi, Elastogenic effects of exogenous hyaluronan oligosaccharides on vascular smooth muscle cells. Biomaterials, 2006. 27 (33): p. 5698-707. TGF-β1 additionally reduced the degradation of elastin through reduction in the levels and activity of elastolytic proteases including matrix metalloproteinase (MMP)-2 and -9; see Dai, J., et al., Overexpression of transforming growth factor-beta1 stabilizes already-formed aortic aneurysms: a first approach to induction of functional healing by endovascular gene therapy. Circulation, 2005. 112 (7): p. 1008-15. Treatment of HDFs with various concentrations of TGF-β1 or TGF-β2 for 24 hours resulted in a dose-dependent elevation in the elastin mRNA steady-state levels, with a maximum 30-fold increase when dosed with 1 ng/ml; see Kähäri, V. M., et al., Transforming growth factor-beta up-regulates elastin gene expression in human skin fibroblasts. Evidence for post-transcriptional modulation. Lab Invest, 1992. 66 (5): p. 580-8. In mammals, 3 Hyaluronic Acid Synthase (HAS) enzymes: HAS-1, 2 and 3 synthesize HA chains of various lengths; see Weigel, P. H., V. C. Hascall, and M. Tammi, Hyaluronan synthases. J Biol Chem, 1997. 272 (22): p. 13997-4000; Itano, N., et al., Three isoforms of mammalian hyaluronan synthases have distinct enzymatic properties. J Biol Chem, 1999. 274 (35): p. 25085-92; and Itano, N. and K. Kimata, Mammalian hyaluronan synthases. IUBMB Life, 2002. 54 (4): p. 195-9. Gene expression of HAS-1 and HAS-2 in the dermis and epidermis is differentially upregulated by TGF-β1; see Stuhlmeier, K. M. and C. Pollaschek, Differential effect of transforming growth factor beta (TGF-beta) on the genes encoding hyaluronan synthases and utilization of the p38 MAPK pathway in TGF-beta-induced hyaluronan synthase 1 activation. J Biol Chem, 2004. 279 (10): p. 8753-60; and Stern, R. and H. I. Maibach, Hyaluronan in skin: aspects of aging and its pharmacologic modulation. Clin Dermatol, 2008. 26 (2): p. 106-22. The mRNA expression of HAS-2 and HAS-3 can be stimulated by Fibroblast Growth Factor (FGF)-7, which activates keratinocyte migration and stimulates wound healing, leading to the accumulation of intermediumte-sized HA in culture medium and within keratinocytes. TGF-β2 and Platelet Derived Growth Factor (PDGF)-BB have been shown to induce expression of HAS-2 as well as hyaluronan synthase by osteoblasts; see Nikitovic, D., et al., Transforming Growth Factor-β as a key molecule triggering the expression of versican isoforms v0 and v1, Hyaluronan Synthase-2 and synthesis of Hyaluronan in Malignant Osteosarcoma cells. IUBMB Life, 2006. 58 (1): p. 47-53.

Thus, the significant upregulation of elastin and hyaluronic acid expression observed in all exposed HDF can be attributed to the presence of TGF-βs, PDGF-BB and FGF-7 present in RD-CLMSC-CM. In addition to the known involvement of growth factors in enhancing elastin and hyaluronic acid expression, the positive implication of growth factors including but not limited to transforming growth factor (TGF) beta1 and TGF beta2, insulin-like growth factor 1 (IGF-1) and platelet-derived growth factor (PDGF, including such as PDGFaa, PDGFbb and/or PDGFab) in hair growth have been found here.

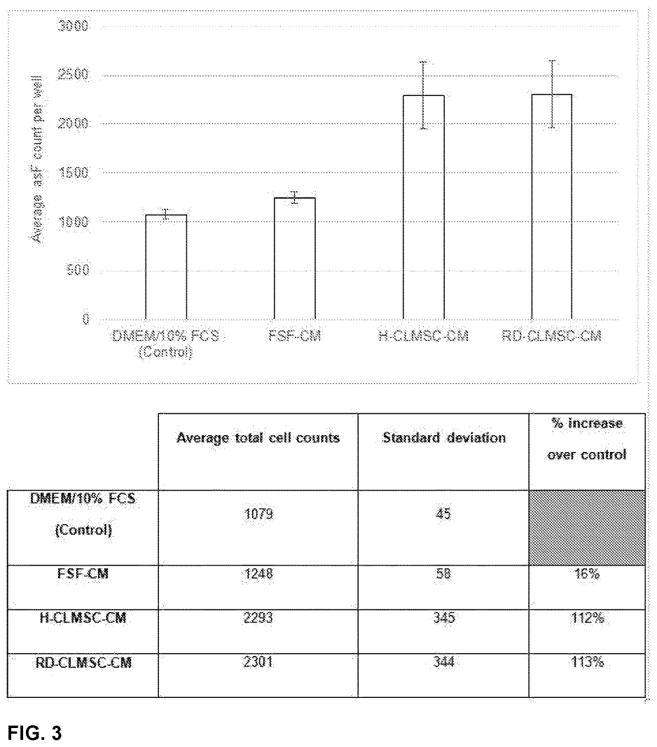

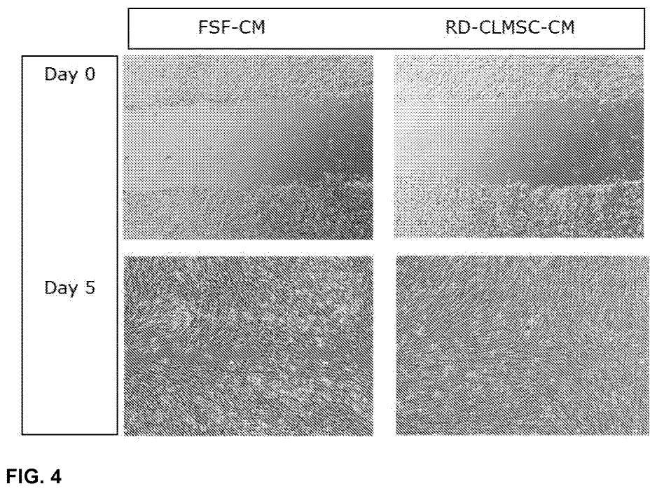

Moreover, it is shown in the Examples that conditioned medium from red deer and human mesenchymal stem cells of the amniotic membrane of umbilical cord applied to human dermal fibroblasts (HDF) significantly increased the proliferation capacity of HDF which could, for example, not achieved with DMEM/10% FCS (control) or Human Foreskin Fibroblast (FSF) (Example 4, FIG. 3). Further, the cellular mobility assessed by an in vitro “scratch” assay showed that the pro-migrative properties of conditioned medium derived from the cultivation of red deer mesenchymal stem cells of the amniotic membrane of umbilical cord are more potent than conditioned medium derived from the cultivation of Human Foreskin Fibroblast (FSF) (Example 5, FIG. 4). The superior proliferative and pro-migrative properties of conditioned medium derived from cultivation of mesenchymal stem cells of the amniotic membrane of umbilical cord (red deer and human) over DMEM/10% FCS (control) or Human Foreskin Fibroblast (FSF) suggests the presence of other proteins and/or growth factors present in such a conditioned medium which might be exerting a positive influence on HDF proliferation. For example, VEGF significantly increased the proliferation capacity of HDF and human lung fibroblasts (see, e.g., Bondarenko, N. A., et al., Effect of Vascular Endothelial Growth Factor and Erythropoietin on Functional Activity of Fibroblasts and Multipotent Mesenchymal Stromal Cells. Bulletin of Experimental Biology and Medicine, 2016. 160 (4): p. 498-501; Larsson-Callerfelt, A.-K., et al., VEGF induces ECM synthesis and fibroblast activity in human lung fibroblasts. European Respiratory Journal, 2017. 50 (suppl 61): p. PA1045) and HGF was shown to induce proliferation of human keratinocytes with the same potency as FGF-7 (see, e.g., Takami, Y., et al., Modulation of hepatocyte growth factor induction in human skin fibroblasts by retinoic acid. Biochimica et Biophysica Acta (BBA)—Molecular Cell Research, 2005. 1743 (1): p. 49-56.).

Thus, the significant enhancement of proliferation and increase in the mobility of HDFs cultured in RD-CLMSC-CM can be attributed to the proliferative and pro-migrative effects of VEGF, TGF-β1 and HGF. In addition to the expression of proliferative factors, conditioned medium derived from the cultivation of mesenchymal stem cells of the umbilical cord may contain anti-apoptotic and maintenance factors to retard cellular senescence and slow down growth arrest and reduce apoptosis. Further, in this context, stimulating hair growth could also be attributed to VEGF and HGF since these growth factors are also known to the skilled person to be positively implicated in hair growth. In this regard, VEGF is known to be involved in hair follicle growth and cycling, wherein hair growth, increase in follicles and hair size is promoted by improved vascularization particularly during the anagen phase of the hair cycle; see Yano et al. Control of hair growth and follicle size by VEGF. Journal of Clinical Investigation, 2001 Feb. 15: 107 (4): 409-417; Lachgar S, Moukadiri H, Jonca F, et al. Vascular endothelial growth factor is an autocrine growth factor for hair dermal papilla cells. Journal of Investigative Dermatology. 1996; 106 (1): 17-23; Kozlowska U, Blume-Peytavi U, Kodelja V, et al. Expression of vascular endothelial growth factor (VEGF) in various compartments of the human hair follicle. Archives of Dermatological Research. 1998; 290 (12): 661-668; Rinaldi F. et al: the role of up-stimulation of growth factors in hair transplantation improve the revascularisation of transplanted hair growth mediated by angiogenesis. Forum 2007:2.

A growth factor is an agent, such as a naturally occurring substance capable of stimulating cellular growth and/or proliferation and/or cellular differentiation. Typically, growth factors are proteins or steroid hormones. “Growth factor” and “factor” may be used interchangeably; however, as used herein, the term “biological factor” is not limited to a growth factor. For example, mesenchymal stem cells of the umbilical cord can produce one or more factors which are involved in, for example, cell proliferation and migration (e.g., Hepatocyte Growth Factor (HGF); Vascular Endothelial Growth Factor (VEGF); Platelet-derived growth factor AA (PDGF-AA); Basic Fibroblast Growth Factor (bFGF); Receptor for urokinase plasminogen activator (UPAR); Intercellular adhesion molecule-1 (ICAM1) and Insulin-Like Growth Factor 1 (IGF1); Interleukin-6 (IL-6); Interleukin-8 (IL-8)); promoting angiogenesis (e.g., Angiopoietin-1 (ANG-1); Hepatocyte Growth Factor (HGF); Vascular Endothelial Growth Factor (VEGF); anti-inflammation (e.g.; Transforming Growth Factor Beta-1 (TGF-β1); Soluble tumor necrosis factor (TNF) receptor 1); promoting elastin and/or hyaluronic acid expression (e.g., Transforming Growth Factor Beta-1 (TGF-β1); Basic Fibroblast Growth Factor (bFGF); Insulin-Like Growth Factor 1 (IGF1)) as well as other factors including Monocyte chemoattractant protein-1 (MCP-1); Tissue inhibitor matrix metalloproteinase 1 (TIMP1) and Tumor necrosis factor receptor superfamily member 10C (TR10C).

Conditioned medium of the invention may comprise at least one and, more typically, a combination of biological factors produced by mesenchymal stem cells of the umbilical cord (e.g., at least one and more typically, a combination of biological factors listed above). For example, they can comprise one or more (at least one) angiogenic cytokines and/or growth factors. They can comprise one or more selected from the group consisting of ANG-1, HGF, VEGF such as VEGFA and/or VEGFB, TGF such as TGF-β1, PDGF such as PDGF-AA or PDGF-BB, bFGF, MCP-1, IL-6, IL-8, TNF, TIMP1, TR10C, UPAR, ICAM1 and IGF1.

In this context, the finding of the present invention is that conditioned medium derived from the cultivation of mesenchymal stem cells of the umbilical cord, wherein the cells inter alia secrete growth factors and other biological factors into the conditioned medium, improves hair growth. By this means, conditioned medium of the present invention is believed to derive high concentration of growth factors and/or other biological factors, which can effectively aid in prolonging the anagen phase, inducing cell growth and cellular proliferation, particularly dermal papilla cell proliferation, as well as suppressing apoptotic cues. In this context, it is noted that cultivation of mesenchymal stem cells of the umbilical cord in the culture medium as described herein provides for the isolation of an extremely homogenous and well-defined mesenchymal stem cell population of umbilical cord tissue (for example, a mesenchymal stem cell population of the Wharton's Jelly or of the amniotic membrane of the umbilical cord) as disclosed in WO 2019/199234 A1 or WO 2018/067071 A1. Particularly, the isolation of a mesenchymal stem cell population of the umbilical cord results in more than 90%, or even 99% or more of the cells that are positive for the three mesenchymal stem cell markers CD73, CD90 and while at the same these stem cells lack expression of CD34, CD45 and HLA-DR (see, e.g., WO 2019/199234 A1 or WO2018/067071 A1), meaning that, e.g., 99% or even more cells of this population express the stem cell markers CD73, CD90 and CD105 while not expressing the markers CD34, CD45 and HLA-DR. In this context, such a mesenchymal stem cell population of the umbilical cord (tissue) fully meet the criteria generally accepted for human mesenchymal stem cells to be used for cellular therapy as defined, for example, by Dominici et al, “Minimal criteria for defining multipotent mesenchymal stromal cells. The International Society for Cellular Therapy position statement”, Cytotherapy (2006) Vol. 8, No. 4, 315-317, Sensebe et al. “Production of mesenchymal stromal/stem cells according to good manufacturing practices: a, review”, Stem Cell Research & Therapy 2013, 4:66), Vonk et al., Stem Cell Research & Therapy (2015) 6:94, or Kundrotas Acta Medica Lituanica. 2012. Vol. 19. No. 2. P. 75-79. Accordingly, this in turn means that conditioned medium which is derived from cultivation of such an extremely homogenous and well-defined cell population is ideal to derive highly homogenous and well-defined components, such as highly concentrated growth factors, in the conditioned medium. Moreover, the use of conditioned medium of the present invention in hair growth therapy has further several advantages over stem-cell based applications since donor-recipient matching problems by stem-cell based hair transplantation can be avoided and conditioned medium is easier to prepare and is cheaper compared to stem-cell based hair therapy.

Moreover, optimal conditions for hair growth therapy and hair transplantation can also be due to a healthy skin condition. In this context, conditioned medium derived from cultivation of mesenchymal stem cells of the umbilical cord has been reported to confer beneficial effects on skin quality and regeneration. For example, the use of conditioned medium showed to optimize the healing process in patients with moderate to severe photodamage (Hoss et al, Red Deer Umbilical Cord-Derived Stem Cell Conditioned Media Combined With Ablative Resurfacing of the Face, Journal of Drugs in Dermatology, Volume 19, Issue 11, November 2020 and to reduce pain and discomfort after laser therapy (Dr. Cheryl Effron, Reduction of Pain and Discomfort post CO2 Fractional Laser resurfacing after the application of umbilical cord lining extract: A Single-Blinded Split-Face Trial, available at https://calecimprofessional.com/pages/clinical-abstract-dr-cheryl-effron). Without wishing to being bound by theory, optimizing healing and reducing pain of damaged skin areas by using conditioned medium can be attributed to the presence of secreted products of mesenchymal stem cells of the umbilical cord contained in the conditioned medium. Such secreted compounds/products are, for example, disclosed in WO2019/199234A1, wherein increased expression and/or secretion of products of the mesenchymal stem cells of the umbilical cord into the medium can cause the effect of inducing or improving wound healing properties. Thus, products secreted by mesenchymal stem cells of the umbilical cord into the conditioned medium can contribute to healthy skin conditions which, as found herein, are also advantageous for an optimal hair growth and/or for healing processes after hair transplantation. The beneficial effect on skin condition by conditioned medium derived from cultivation of mesenchymal stem cells of the umbilical cord can be attributed inter alia to immunomodulatory properties by conditioned medium, such immunomodulatory properties are also known to be achieved by mesenchymal stem cells of the umbilical cord. In accordance with these findings, exposure of conditioned medium derived from the cultivation of mesenchymal stem cells of the umbilical cord under the scalp of a subject has been found herein to reduce inflammation. See Example 9 herein, wherein production of tumor necrosis factor-alpha (TNF-alpha), a key inflammatory cytokine produced by macrophages/monocytes during acute inflammation and responsible for various signaling pathways in cells such as those resulting in necrosis or apoptosis, was significantly downregulated. Thus, in addition to stimulating and/or promoting hair growth and/or hair regeneration, the invention has the added advantage that inflammation can be reduced by the conditioned medium and promote cell growth and cell proliferation.

The conditioned medium derived from cultivation of mesenchymal stem cells of the umbilical cord that is being rendered suitable for hair growth (e.g., by inducing stimulating hair growth in a patient suffering from hair thinning/hair loss) may be derived from cultivation of any suitable mesenchymal stem cell population of the umbilical cord (tissue) known in the art. For example, the mesenchymal stem cells of the umbilical cord may be derived from any mammalian tissue or compartment/body part known to contain mesenchymal stem cells of the umbilical cord. In illustrative examples, the mesenchymal stem cells may be mesenchymal stem cells of the amniotic membrane of umbilical cord. The effects of promoting hair growth and preventing hair-loss are achieved, e.g., because these stem cells secrete growth factors and other biological factors associated with hair growth, such as by producing dermal papilla cells and/or increasing the number and size of hair follicles. For example, biological factors that may be in the conditioned medium include but are not limited to growth factors, nucleic acids such as mRNA or DNA, exosomes, hormones, cytokines, extracellular matrix, proteins, vesicles, antibodies, chemokines, receptors, inhibitor, and granules. Such conditioned medium, and combinations of any of the biological factors comprised therein, including but not limited to growth factors, may be used in the method of the present invention.

The conditioned medium of the present invention used for treatment may, for example, be applied undiluted or diluted. When used in diluted form, the conditioned medium may be used at a final concentration of, for example, at about 10% to about 90% or about 10% to about 80% including at a final concentration of about 20%, about 25%, about 30%, about 35%, about 40%, about 45%, about 50%, about 55%, about 60%, about 65%, or about 70% (v/v of the total volume of the composition that contains the conditioned medium). For this purpose, the conditioned medium of the present invention can be diluted in (formulated with) any suitable diluent/carrier medium. The carrier medium may be a liquid, a gel or a cream-like formulation. For example, the conditioned medium may be diluted in a carrier medium such as a physiological buffer such as PBS or saline, water, an aqueous solution of an extracellular matrix component, a basal medium, or a cream formulation to name only a few suitable media. A basal medium as used herein refers to a mixture containing saccharides, amino acids, water, etc., required for the survival of a cell and include commercial prepared medium such as but not limited to Dulbecco's Modified Eagle's Medium (DMEM), Endothelial differentiation medium (EDM), Minimal Essential Medium (MEM), Basal Medium Eagle (BME), RPMI 1640, F-10, F-12, α-Minimal Essential Medium (α-MEM), Glasgow's Minimal Essential Medium (G-MEM), and Iscove's Modified Dulbecco's Medium. Examples of aqueous solutions of extracellular matrix components are aqueous hyaluronic acid, aqueous solutions of collagen or aqueous solutions of fibronectin, all of which are commercially available for use in cosmetics. An illustrative example of such a composition of the invention that can be used for inducing, stimulating and/or promoting hair growth and/or hair regeneration as described here is a solution (liquid composition) that comprises about 90 to about 10% (v/v) of conditioned medium and the remainder (i.e., about 10 to about 90% (v/v) being aqueous hyaluronic acid. In some other examples, the conditioned medium of the present invention may be comprised in a solution that comprises DMEM as basal medium. Also in such a solution, the conditioned medium be present, in a range of, for example, about 90% to about 10% (v/v) and the remainder being DMEM. The conditioned medium of the invention may further comprise an antioxidant such as a soluble antioxidant. The water-soluble antioxidant may be added to the medium to prevent oxidation damage. Examples of suitable water-soluble antioxidants include glutathione, uric acid, Trolox or Allicidin to mention only a few.

The conditioned medium of the present invention may also be formulated in a form that improves cutaneous absorption of the conditioned medium after topical administration to the skin/scalp of a subject. For this purpose, the conditioned medium may be encapsulated in a liposomal formulation. An illustrative example of such a liposomal formulation are liposomes that are made from a phospholipid mixture. Such a phospholipid mixture for cosmetic use is, for example, the liposomal delivery system named Pro-Lipo™ Neo of Lucas Meyer Cosmetics, which is a ready-to-use liposome delivery system that allow to encapsulate the conditioned medium for topical administration (see Example 12). A composition for topical administration as described herein may, for example, contain about 30 to 70% (v/v) conditioned medium and about 20% (v/v) of the Pro-Lipo™ Neo (Liposomal Mixture), with the remainder/balance being any suitable aqueous solution.

The conditioned medium of the invention may be derived, after cultivation of the mesenchymal stem cells in the medium that comprises DMEM, F12, M171 and FBS, from cultivation of the mesenchymal stem cells of the umbilical cord in the above-mentioned further culture medium. This further culture medium may be any (further) basic culture medium suitable for animal cell growth, and in particular a basal medium suitable for cultivation of mesenchymal stem cells. This further culture medium may be a serum-free medium. Non-limiting examples of such a serum-free medium include Minimal Essential Medium (MEM), Dulbecco modified Eagle Medium (DMEM), Roswell Park Memorial Institute Medium (RPMI), and Keratinocyte Medium (KM), KBM (Keratinocyte Basal medium), EpiLife KM (Keratinocyte-EpiLife medium) etc. The further culture medium may alternatively also contain serum. As an example of such a serum containing medium, the conditioned medium to be used may be one that is described and usually used for the isolation and cultivation of a mesenchymal stem cell population of the amniotic membrane of umbilical cord such as medium PTT-4. This medium PTT-4 consists of 90% (v/v) CMRL1066, and 10% (v/v) FBS) and has been used in US patent application US 2008/0248005 and the corresponding International patent application WO2007/046775 for the isolation and cultivation of a mesenchymal stem cell population of the amniotic membrane of umbilical cord which was shown to have excellent wound healing properties in US patent application US 2008/0248005 and International patent application WO2007/046775. In some examples, a cell culture medium as described herein such as a culture medium comprising DMEM (Dulbecco's modified eagle medium), F12 (Ham's F12 Medium), M171 (Medium 171) and FBS (Fetal Bovine Serum) may be used for cultivation of mesenchymal stem cells of the umbilical cord, wherein the cell culture medium is removed after cultivation, e.g., once the cells reach full confluency, and replaced by another culture medium/solution such as a culture medium solution comprising, for example, DMEM which may comprise an antioxidant ingredient. Generally, during the optional cultivation step in the further medium of the method of preparing a conditioned medium as described here, the mesenchymal stem cells can be cultivated for any suitable period of time. Such a suitable period of time may, for example, be a period of 1 day, 2 days, 3 days, 4 days, 5 days, 6 days, 7 days or even longer, for example, a period of up to 10 days or 14 days, if desired. In this context, it is noted for clarity, that both, the conditioned medium obtained in the first cultivation step and also the conditioned medium obtained in the second (further) cultivation step, can be used for inducing, stimulating and/or promoting hair growth and/or hair regeneration as described herein.

The mesenchymal stem cells of the umbilical cord (tissue) used herein for the preparation of conditioned medium may be (derived) from any compartment of umbilical cord tissue that contains mesenchymal stem cells. The mesenchymal stem cell population may be a mesenchymal stem cell population of the amnion (AM), a perivascular (PV) mesenchymal stem cell population, a mesenchymal stem cell population of Wharton's jelly (WJ), a mesenchymal stem cell population of the amniotic membrane of umbilical cord (also referred to as cord lining mesenchymal cells) but also a mixed mesenchymal stem cell population of the umbilical cord (MC), meaning a population of mesenchymal stem cells that includes stem cells of two or more of these compartments. In some examples, the mesenchymal stem cells are of the amniotic membrane of the umbilical cord. Mesenchymal stem cells of these compartments and the isolation therefrom are known to the person skilled in the art and are described, for example, by Subramanian et al “Comparative Characterization of Cells from the Various Compartments of the Human Umbilical Cord Shows that the Wharton's Jelly Compartment Provides the Best Source of Clinically Utilizable Mesenchymal Stem Cells”, PLOS ONE 10 (6): e0127992, 2015 and the references cited therein, Van Pham et al. “Isolation and proliferation of umbilical cord tissue derived mesenchymal stem cells for clinical applications”, Cell Tissue Bank (2016) 17:289-302, 2016. A mixed mesenchymal stem cell population of the umbilical cord can, for example, be obtained by removing the arteries and veins from the umbilical cord tissue, cutting the remaining tissue and the Wharton's jelly into piece and cultivating the umbilical cord tissue (by tissue explant) in a culture medium, such as PTT-6. A mixed mesenchymal stem cell population of the umbilical cord may also be obtained by culturing entire umbilical cord tissue with intact umbilical vessels as tissue explant under the conditions (cultivation in serum-supplemented DMEM with 10% fetal bovine serum, 10% horse serum, and 1% Penicillin/Streptomycin) as described by Schugar et al. “High harvest yield, high expansion, and phenotype stability of CD146 mesenchymal stromal cells from whole primitive human umbilical cord tissue. Journal of biomedicine & biotechnology. 2009; 2009:789526”. In this context, it is noted that a mesenchymal stem cell population of the cord-placenta junction can be isolated as described by Beeravolu et al. “Isolation and Characterization of Mesenchymal Stromal Cells from Human Umbilical Cord and Fetal Placenta.” J Vis Exp. 2017; (122): 55224.

In accordance with the above, it is noted here that conditioned medium is derived from cultivation of mesenchymal stem cells of umbilical cord tissue, wherein the stem cells can be cultivated in a culture medium comprising DMEM (Dulbecco's modified eagle medium), F12 (Ham's F12 Medium), M171 (Medium 171) and FBS (Fetal Bovine Serum) and which can be isolated from its natural environment prior to cultivation in a culture medium as described herein. Such an approach is in particular used for a mesenchymal stem cell population of the umbilical cord. Such a stem cell population, say a mesenchymal stem cell population of Wharton's jelly, may first be isolated as described above by Subramanian et al, 2015, PLOS ONE, supra or International Patent application WO 2004/072273 “Progenitor Cells From Wharton's Jelly Of Human Umbilical Cord” and then be subjected to cultivation of the isolated mesenchymal stem cell population in the culture medium that comprises DMEM (Dulbecco's modified eagle medium), F12 (Ham's F12 Medium), M171 (Medium 171) and FBS (Fetal Bovine Serum). Also a placental mesenchymal stem cell population may be isolated from placenta as described in European patent application EP1 288 293, Talwadekar et al, “Cultivation and Cryopreservation of Cord Tissue MSCs with Cord Blood AB Plasma” Biomed Res J 2014; 1 (2): 126-136, Talwadekar et al, “Placenta-derived mesenchymal stem cells possess better immunoregulatory properties compared to their cord-derived counterparts—a paired sample study” Scientific Reports 5:15784 (2015), or Beeravolu et al. “Isolation and Characterization of Mesenchymal Stromal Cells from Human Umbilical Cord and Fetal Placenta.” J Vis Exp. 2017; (122): 55224, for example, and subsequently cultivated in the culture medium as described herein.

In this context, it is noted that the culture medium as described herein therefore also allows the isolation of a mesenchymal stem cell population (also referred here as “mesenchymal stem cells”) from its natural environment. Accordingly, the culture medium as described herein allows also isolation of a mesenchymal stem cell population under conditions that allow cell proliferation of the mesenchymal stem/progenitor cells without differentiation of the mesenchymal stem/progenitor cells. In some examples, the cell culture medium(s) used for isolation and/or cultivation of mesenchymal stem cells of the umbilical cord may be the medium used for cultivation of the cells in order to produce the conditioned medium of the present invention.

In accordance with the above disclosure, it is noted here that such a mesenchymal stem cell population described herein can be isolated and cultivated (i.e., are derived) from any umbilical cord tissue as long as the umbilical cord tissue contains the amniotic membrane (which is also referred to as “cord lining”). Accordingly, the mesenchymal stem cell population can be isolated from (pieces of) the entire umbilical cord as described in the experimental section of the present application. This umbilical cord tissue may thus contain, in addition to the amniotic membrane, any other tissue/component of the umbilical cord. As shown, for example, in FIG. 16 of US patent application 2006/0078993 or International patent application WO2006/019357, the amniotic membrane of the umbilical cord is the outmost part of the umbilical cord, covering the cord. In addition, the umbilical cord contains one vein (which carries oxygenated, nutrient-rich blood to the fetus) and two arteries (which carry deoxygenated, nutrient-depleted blood away from the fetus). For protection and mechanical support these three blood vessels are embedded in the Wharton's jelly, a gelatinous substance made largely from mucopolysaccharides. Accordingly, the umbilical cord tissue used herein can also comprise this one vein, the two arteries and the Wharton's jelly. The use of such an entire (intact) section of the umbilical cord has the advantage that the amniotic membrane does not need to be separated from the other components of the umbilical cord. This reduces the isolation steps and thus makes the method of the present invention, simpler, faster, less error prone and more economical. The isolation of the mesenchymal stem cells can thus start by tissue explant, which may be followed by subsequent subculturing (cultivation) of the isolated mesenchymal stem cells if greater amounts of the mesenchymal stem cells are desired. Alternatively, it is also possible to first separate the amniotic membrane from the other components of the umbilical cord and isolate the mesenchymal cord lining stem cells from the amniotic membrane by cultivation of the amniotic membrane in a culture medium as described herein. This cultivation can also be carried out by tissue explant, optionally followed by subculturing of the isolated mesenchymal stem cells.

In this context, the term “tissue explant” or “tissue explant method” is used in its regular meaning in the art to refer a method in which a tissue (for example, umbilical cord tissue), once being harvested, or a piece of the tissue is being placed in a cell culture dish containing culture (growth) medium and by which over time, the stem cells migrate out of the tissue onto the surface of the dish. These primary stem cells can then be further expanded and transferred into fresh dishes through micropropagation (subculturing) as also described here. In this context, it is noted that in the first step of isolating/obtaining a mesenchymal stem cell population of the umbilical cord such as amniotic membrane or Wharton's jelly mesenchymal stem cells, a master cell bank of the isolated mesenchymal stem cells is obtained, while in the subsequent subculturing a working cell bank can be obtained. If conditioned medium derived from cultivation of mesenchymal stem cells of the umbilical cord of the invention is used for application in promoting hair growth, a cell population of the working cell bank will be typically used for this purpose. Both the stem cell population of the isolation step (which may make up the master cell bank) and the stem cell population of the subculturing step (which may make up the working cell bank) can, for example, be stored in cryopreserved form.

By “inducing, stimulating and/or promoting hair growth and/or hair regeneration” is meant herein the ability of the conditioned medium to increase and/or start (induce) hair growth from hair follicles, particularly in patients having underactive or dormant hair follicles and/or patients suffering from hair thinning/hair loss such as androgenic alopecia. The ability to increase and/or start hair growth can be attributed to the presence of growth factors, which, as explained above, are known to the skilled person to be involved in inducing, stimulating and/or promoting hair growth. In the experimental examples herein, the secretion of growth factors which are positively implicated with “Inducing, stimulating and/or promoting hair growth and/or hair regeneration” and are present in conditioned medium derived from mesenchymal stem cells of the umbilical cord are assessed relative to conditioned medium of Human Foreskin Fibroblast (FSF) and DMEM/10% FCS (control). In this sense, cultivating human dermal fibroblasts (HDF) in conditioned medium derived from cultivation of mesenchymal stem cells of the umbilical cord demonstrated superior proliferative and pro-migrative properties of human dermal fibroblasts compared to cultivation in DMEM/10% FCS (control) or Human Foreskin Fibroblast (FSF) which can be attributed to that the mesenchymal stem cells of the umbilical cord secrete a high amount (corresponding to a high secretion level or high concentration) of growth factors into the supernatant/culture medium compared to cultivation of FSF. The secretion of growth factors into the culture medium (and thus the growth factors contained in the conditioned medium of the present invention) can be measured/determined with any suitable method for example, by measuring the amount of growth factors by means of commercially available antibodies/immunoassays (cf., the Experimental Section). Such measurements can be made in an automated fashion, using, for example a system such as the FLEXMAP 3D system (Luminex Corporation, Austin, Texas, USA). The ability of the conditioned medium described herein to induce, stimulate and/or promote hair growth and/or hair regeneration can also be determined by cellular assays such as the examining the growth promotion effect of conditioned medium on cultured human follicle dermal papilla cells as described herein (see Example 9) or by administering the conditioned medium to patients being diagnosed with hair loss and examining, for example, its effect on the number of follicular units per square centimeter, the intra-follicular distance or the total hair count per square centimeter as described herein (see Example 11).

By “Norwood Classification or Scale” or “Norwood-Hamilton Classification or Scale” is meant the classification that O'Tar Norwood introduced to classify the stages of male pattern baldness. The Norwood classification/scale is the most widely used classification for hair loss in men and defines two major patterns and several less common types (see, for example, Gupta & Mysore, Classifications of Patterned Hair Loss: A Review, J Cutan Aesthet Surg. 2016 January-March; 9 (1): 3-12). Norwood observed that thinning starts in the temples as well as the crown/vertex and slowly progresses to encompass the entire top of the scalp. This classification is thus based on this pattern and is also shown in FIG. 15. The grades of hair loss according to the Norwood scale are the following (Roman and Arabic numerals are used interchangeably herein when referring to the Norwood scale):

Type I: There is minimal or no recession of the hairline.
Type II: There are triangular, usually symmetrical, areas of recession at the frontotemporal hairline.
Type III: This represents the minimal extent of hair loss sufficient to be considered as baldness according to Norwood. There are deep symmetrical recession at the temples that are bare or only sparsely covered by hair. In Type Ill vertex, the hair loss is primarily from the vertex with limited recession of the frontotemporal hairline that does not exceed the degree of recession seen in Type III.
Type IV: The frontotemporal recession is more severe than in Type Ill and there is sparse hair or no hair on the vertex. The two areas of hair loss are separated by a band of moderately dense hair that extends across the top. This band connects with the fully haired fringe on the sides of the scalp.
Type V: The vertex hair loss region is still separated from the frontotemporal region but it is less distinct. The band of hair across the crown is narrower and sparser and the vertex and frontotemporal regions of hair loss are bigger.
Type VI: The bridge of hair that crosses the crown is gone with only sparse hair remaining. The frontotemporal and vertex regions are joined together and the extent of hair loss is greater.
Type VII: The most severe form of hair loss and only a narrow band of hair in a horseshoe shape remains on the sides and back of the scalp. This hair is usually not dense and may be quite fine.
The Norwood scale also defines a Type A variant from his standard classification system, which is distinguished by two major features and two minor features.
The major features are: 1) the anterior border of the hairline progresses to the rear without leaving an island of hair in the mid-frontal region and 2) there is no simultaneous development of a bald area on the vertex. Instead, the frontal hairline recession keeps progressing to the rear of the scalp.
The minor features are: 1) there is a persistent sparse hair scattering in the area of hair loss and 2) the horseshoe-shaped fringe areas of hair that remain on the side and back of the scalp tend to be wider and reach higher on the head compared to Norwood's standard. The various Type A variants described by Norwood are as follows:
Type IIA: The hairline is anterior to the coronal plane 2 cm anterior to the external auditory meatus.
Type IIIA: The hairline has receded back to a point between the limit of Type IIA and the level of the external auditory meatus.
Type IVA: The hairline has receded beyond the external auditory meatus but has not reached the vertex.
Type VA: The area of denudation includes the vertex. Hair loss more severe than Type VA cannot be distinguished from Types VI or VII.
In this context, it is noted that it has been found herein that conditioned medium as described here can be preferably used to treat male hair loss, wherein the male has been diagnosed from hair loss of Type I, Type II, Type III, Type IV, Type V or Type VI hair loss according to the Norwood classification. Or in any words, hair loss of any of Type I, Type II, Type III, Type IV, Type V or Type VI according to the Norwood classification can be treated with conditioned medium of the present invention.

By “Ludwig classification or scale” is meant the classification that E. Ludwig introduced in 1979 to classify female pattern hair loss or baldness (androgenic alopecia). It ranges from Type/Grade I to Type/Grade III that are defined as follows (see also FIG. 16), with the terms “Type” and Grade” being used interchangeably herein. Roman and Arabic numerals are used interchangeably herein when referring to the Ludwig scale.

Grade I: Perceptible thinning of the hair on the crown, limited in the front by a line situated 1-3 cm behind the frontal hair line. As shown in FIG. 16, grade I is further classified into grade Ia, Ib, Ic and Id and grade II is further classified into grade IIa and IIb).
Grade II: Pronounced rarefaction of the hair on the crown within the area seen in Grade I.
Grade III: Full baldness (total denudation) within the area seen in Grades I and II.
Grade I begins with thinning on the top of the head. In grade II the scalp starts to show. All of the hair at the crown of the head may be lost when the hair loss progresses to grade III.
In this context, it is noted that it has been found herein that conditioned medium as described here can be preferably used to treat female hair loss, wherein the female has been diagnosed with hair loss of Type I (including Type Ia, Type Ib, Type Ic and Type Id), Type II (Type IIa and Type IIb) or Type III hair loss according to the Ludwig classification. Or in any words, hair loss of any of Type I, (including Type Ia, Type Ib, Type Ic and Type Id), Type II (Type IIa and Type IIb) or Type III hair loss according to the Ludwig classification can be treated with conditioned medium of the present invention.

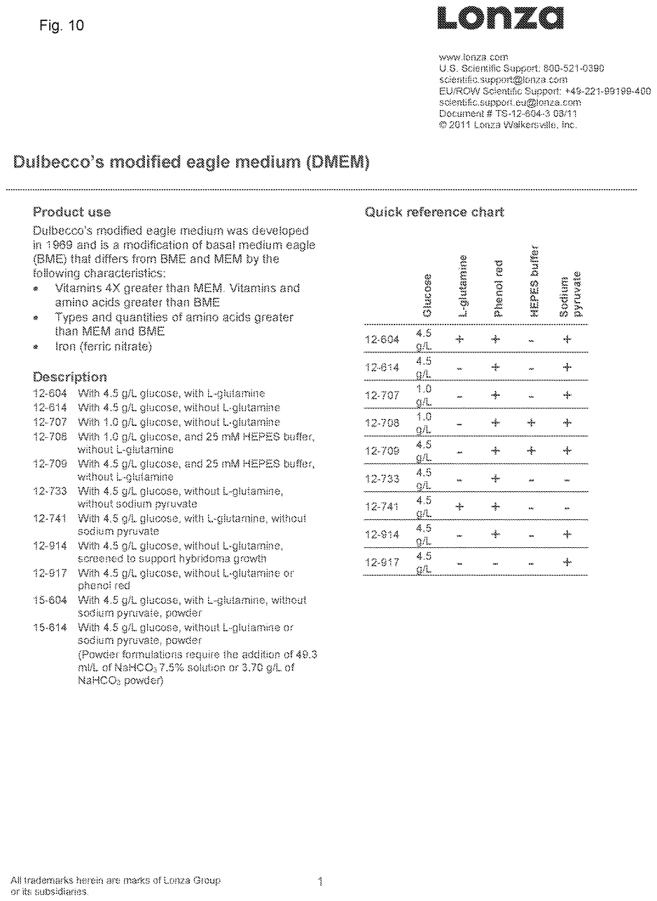

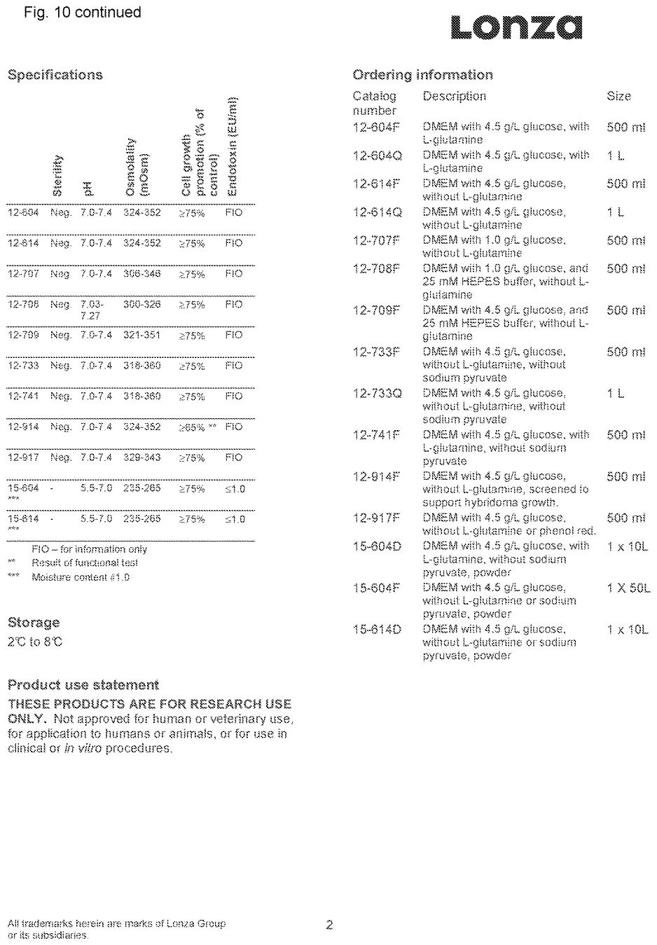

By “DMEM” is meant Dulbecco's modified eagle medium which was developed in 1969 and is a modification of basal medium eagle (BME) (cf. FIG. 10 showing the data sheet of DMEM available from Lonza). The original DMEM formula contains 1000 mg/L of glucose and was first reported for culturing embryonic mouse cells. DMEM has since then become a standard medium for cell culture that is commercially available from various sources such as ThermoFisher Scientific (catalogue number 11965-084), Sigma Aldrich (catalogue number D5546) or Lonza, to name only a few suppliers. Thus, any commercially available DMEM can be used in the present invention. In preferred embodiments, the DMEM used herein is the DMEM medium available from Lonza under catalog number 12-604F. This medium is DMEM supplemented with 4.5 g/L glucose and L-glutamine). In another preferred embodiment the DMEM used herein is the DMEM medium of Sigma Aldrich catalogue number D5546 that contains 1000 mg/L glucose, and sodium bicarbonate but is without L-glutamine.

By “FI2” medium is meant Ham's F12 medium. This medium is also a standard cell culture medium and is a nutrient mixture initially designed to cultivate a wide variety of mammalian and hybridoma cells when used with serum in combination with hormones and transferrin (cf. FIG. 11, showing the data sheet of Ham's F12 medium from Lonza). Any commercially available Ham's F12 medium (for example, from ThermoFisher Scientific (catalogue number 11765-054), Sigma Aldrich (catalogue number N4888) or Lonza, to new only a few suppliers) can be used in the present invention. In preferred embodiments, Ham's F12 medium from Lonza is used.

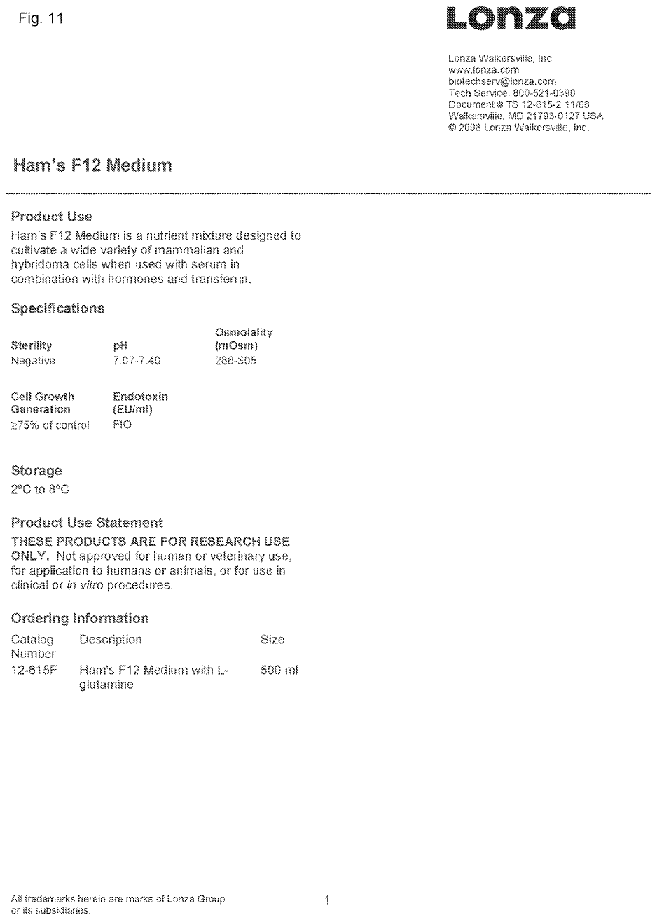

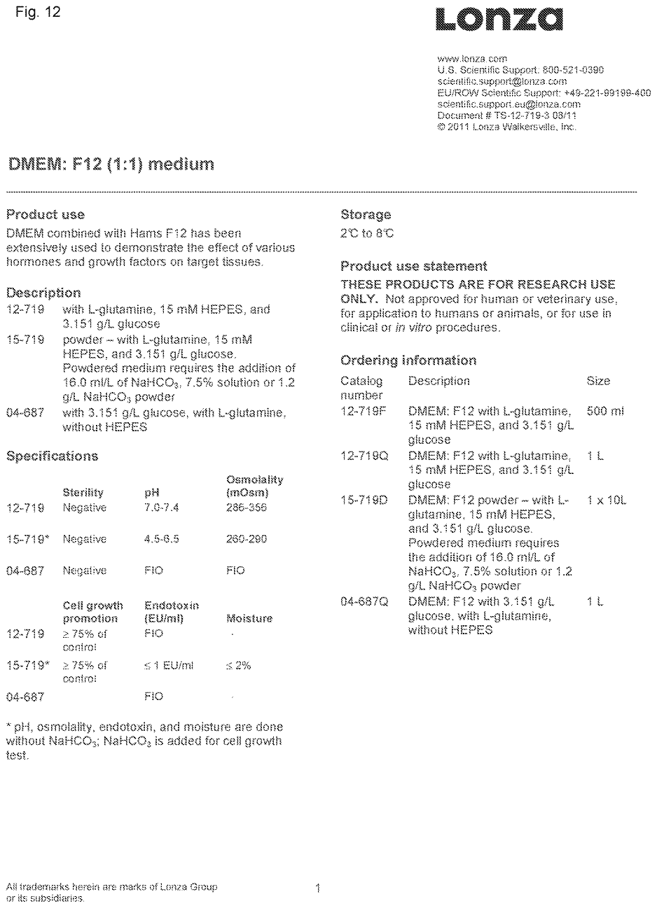

By “DMEM/F12” or “DMEM: F12” is meant a 1:1 mixture of DMEM with Ham's F12 culture medium (cf. FIG. 12 showing the data sheet for DMEM: F12 (1:1) medium from Lonza). Also DMEM/F12 (1:1) medium is a widely used basal medium for supporting the growth of many different mammalian cells and is commercially available from various supplier such as ThermoFisher Scientific (catalogue number 11330057), Sigma Aldrich (catalogue number D6421) or Lonza. Any commercially available DMEM: F12 medium can be used in the present invention. In preferred embodiments, the DMEM: F12 medium used herein is the DMEM/F12 (1:1) medium available from Lonza under catalog number 12-719F (which is DMEM: F12 with L-glutamine, 15 mM HEPES, and 3.151 g/L glucose).

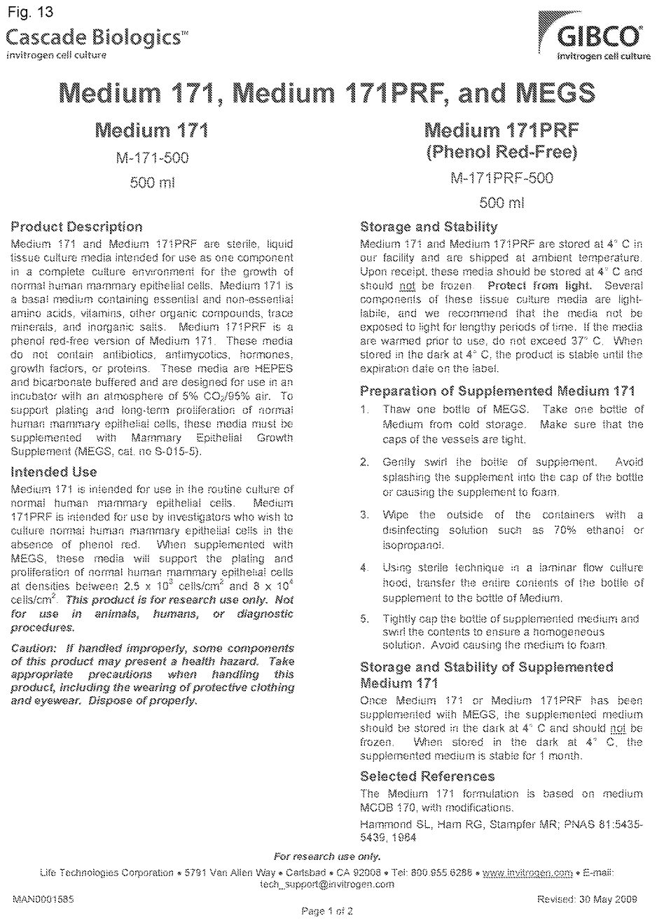

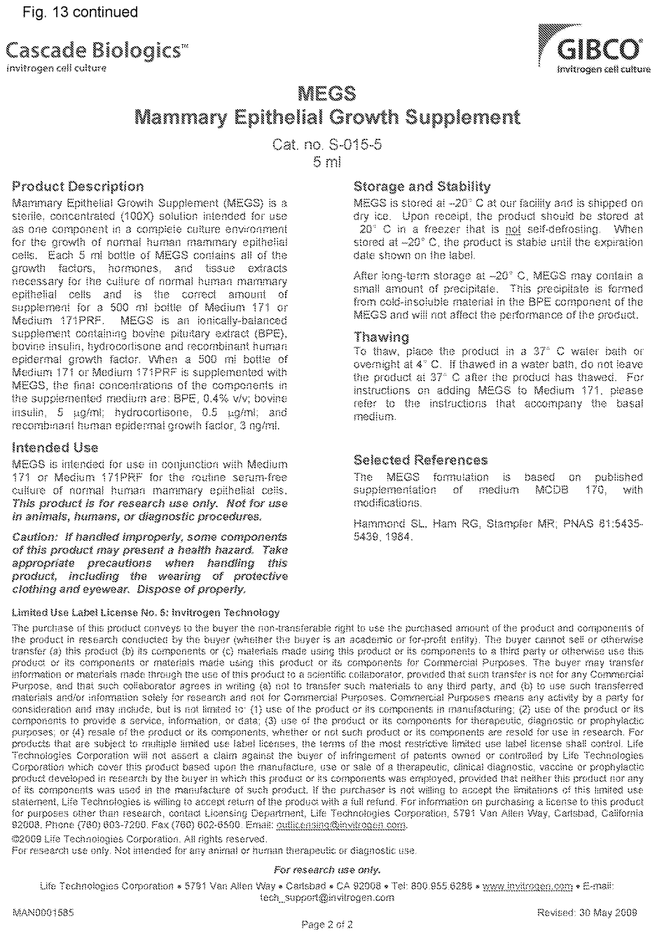

By “M171” is meant culture medium 171, which has been developed as basal medium for the culture of for the growth of normal human mammary epithelial cells (cf. FIG. 13 showing the data sheet for M171 medium from Life Technologies Corporation). Also this basal medium is widely used and is commercially available from supplier such as ThermoFisher Scientific or Life Technologies Corporation (catalogue number M171500), for example. Any commercially available M171 medium can be used in the present invention. In preferred embodiments, the M171 medium used herein is the M171 medium available from Life Technologies Corporation under catalogue number M171500.

By “FBS” is meant fetal bovine serum (that is also referred to as “fetal calf serum”), i.e., the blood fraction that remains after the natural coagulation of blood, followed by centrifugation to remove any remaining red blood cells. Fetal bovine serum is the most widely used serum-supplement for in vitro cell culture of eukaryotic cells because it has a very low level of antibodies and contains more growth factors, allowing for versatility in many different cell culture applications. The FBS is preferably obtained from a member of the International Serum Industry Association (ISIA) whose primary focus is the safety and safe use of serum and animal derived products through proper origin traceability, truth in labeling, and appropriate standardization and oversight. Suppliers of FBS that are ISIA members include Abattoir Basics Company, Animal Technologies Inc., Biomin Biotechnologia LTDA, GE Healthcare, Gibco by Thermo Fisher Scientific and Life Science Production, to mention only a few. In currently preferred embodiments, the FBS is obtained from GE Healthcare under catalogue number A15-151.

The culture medium as described herein may comprise for deriving the conditioned medium from cultivation of the mesenchymal stem cells of the umbilical cord DMEM in a final concentration of about 55 to 65% (v/v), F12 in a final concentration of about 5 to 15% (v/v), M171 in a final concentration of about 15 to 30% (v/v) and FBS in a final concentration of about 1 to 8% (v/v). The value of “% (v/v)” as used herein refers to the volume of the individual component relative to the final volume of the culture medium. This means, if DMEM is, for example, present in the culture medium a final concentration of about 55 to 65% (v/v), 1 liter of culture medium contains about 550 to 650 ml DMEM. In other embodiments, the culture medium may comprise DMEM in a final concentration of about 57.5 to 62.5% (v/v), F12 in a final concentration of about 7.5 to 12.5% (v/v), M171 in a final concentration of about 17.5 to 25.0% (v/v) and FBS in a final concentration of about 1.75 to 3.5% (v/v). In further embodiments, the culture medium may comprise DMEM in a final concentration of about 61.8% (v/v), F12 in a final concentration of about 11.8% (v/v), M171 in a final concentration of about 23.6% (v/v) and FBS in a final concentration of about 2.5% (v/v).

In addition to the above-mentioned components, the culture medium may comprise supplements that are advantageous for cultivation of the mesenchymal stem cells of the umbilical cord and for deriving conditioned medium for inducing, stimulating and/or promoting hair growth and/or hair regeneration. The culture medium as described herein may, for example, comprises Epidermal Growth Factor (EGF). If present, EGF may be present in the culture medium in a final concentration of about 1 ng/ml to about 20 ng/ml. In some of these embodiments, the culture medium may comprise EGF in a final concentration of about 10 ng/ml.

The culture medium as described herein may also comprise insulin. If present, insulin may be present in a final concentration of about 1 μg/ml to 10 μg/ml. In some of these embodiments, the culture medium may comprise Insulin in a final concentration of about 5 μg/ml.

The culture medium may further comprise at least one of the following supplements: adenine, hydrocortisone, and 3,3′,5-Triiodo-L-thyronine sodium salt (T3). In such embodiments, the culture medium may comprise all three of adenine, hydrocortisone, and 3,3′,5-Triiodo-L-thyronine sodium salt (T3). In these embodiments, the culture medium may comprise adenine in a final concentration of about 0.05 to about 0.1 μg/ml adenine, hydrocortisone in a final concentration of about 1 to about 10 μg/ml hydrocortisone and/or 3,3′,5-Triiodo-L-thyronine sodium salt (T3) in a final concentration of about 0.5 to about 5 ng/ml.

As described herein, umbilical cord tissue may be cultured till a suitable number of (primary) mesenchymal stem cells such as cord lining stem cells, Wharton's Jelly or placental stem cells have outgrown from the tissue. Typically, the umbilical cord tissue is cultivated until cell outgrowth of the mesenchymal stem cells of the respective tissue reaches about 70 to about 80% confluency. It is noted here that the term “confluency” or “confluence” is used in its regular meaning in the art of cell culture and is meant as an estimate/indicator of the number of adherent cells in a culture dish or a flask, referring to the proportion of the surface which is covered by cells. For example, 50 percent confluence means roughly half of the surface is covered and there is still room for cells to grow. 100 percent confluence means the surface is completely covered by the cells, and no more room is left for the cells to grow as a monolayer.

Once a suitable number of primary cells (mesenchymal stem cells) have been obtained from the respective tissue by tissue explant, the mesenchymal stem cells are removed from the cultivation container used for the cultivation. By so doing, a master cell bank containing the (primary) isolated mesenchymal stem cells of for example, the umbilical cord or the placenta can be obtained. Typically, since such mesenchymal stem cells are adherent cells, harvesting the cells is carried out using standard enzymatic treatment. For example, the enzymatic treatment may comprise trypsination as described in International US patent application 2006/0078993, International patent application WO2006/019357 or International patent application WO2007/046775, meaning outgrowing cells can be harvested by trypsinization (0.125% trypsin/0.05% EDTA) for further expansion. If the harvested mesenchymal stem cells are, for example, used for generating a master cell bank, the cells can also be cryo-preserved and stored for further use as explained herein below.

Once being harvested, the mesenchymal stem cells can be transferred to a cultivation container for subculturing. Subculturing or culturing (both terms are used interchangeable hereinafter) will be also be carried out if a mesenchymal stem cell population of the umbilical cord is employed that has been isolated from its natural environment earlier. The subculturing can also be started from frozen primary cells, i.e., from the master cell bank. For subculturing any suitable amount of cells can be seeded in a cultivation container such as cell culture plate. The mesenchymal cells can, for this purpose, be suspended in a suitable medium (most conveniently, the culture medium as described herein) for subculturing at a concentration of, for example, about 0.5×10⁶ cells/ml to about 5.0×10⁶ cells/ml. For example, the cells are suspended for subcultivation at a concentration of about 1.0×10⁶ cells/ml. The subculturing can be carried by cultivation either in simple culture flasks but also, for example, in a multilayer system such as CellStacks (Corning, Corning, NY, USA) or Cellfactory (Nunc, part of Thermo Fisher Scientific Inc., Waltham, MA, USA) that can be stacked in incubators. Alternatively, the subculturing can also be carried out in a closed self-contained system such as a bioreactor. Different designs of bioreactors are known to the person skilled in the art, for example, parallel-plate, hollow-fiber, or micro-fluidic bioreactors. See, for example, Sensebe et al.” Production of mesenchymal stromal/stem cells according to good manufacturing practices: a review “, supra. An illustrative example of a commercially available hollow-fiber bioreactor is the Quantum® Cell Expansion System (Terumo BCT, Inc). that has, for example, been used for the expansion of bone marrow mesenchymal stem cells for clinical trials (cf., Hanley et al, Efficient Manufacturing of Therapeutic Mesenchymal Stromal Cells Using the Quantum Cell Expansion System, Cytotherapy. 2014 August; 16 (8): 1048-1058). Another example of commercially available bioreactors that can be used for the subculturing of the mesenchymal stem cell population as described herein is the Xuri Cell Expansion System available from GE Heathcare. The cultivation of the mesenchymal stem cell population in an automated system such as the Quantum® Cell Expansion System is of particular benefit if a working cell bank is to be produced under GMP conditions and a high number of cells is wanted.

The subculturing of the mesenchymal stem cells of the invention can take place in a culture medium as described herein. Accordingly, the culture medium can be used both for the isolation of the mesenchymal stem cell population, for example, from the amniotic membrane or from Wharton's jelly of umbilical cord and the subsequent cultivation of the isolated primary cells by cultivation. Also for the cultivation, the mesenchymal stem cells can be cultured till a suitable amount of cells have grown. In some examples, the mesenchymal stem cells are cultured till the mesenchymal stem cells reach about 70% confluency, about 80% confluency, about 90% confluency, about 95% confluency, to about 96% or even 100% confluency.

The isolation/cultivation of the population of mesenchymal stem cell population can be carried out under standard condition for the cultivation of mammalian cells. Typically, the method of isolating the population of the mesenchymal stem cells is carried out under conditions (temperature, atmosphere) that are normally used for cultivation of cells of the species of which the cells are derived. For example, human umbilical cord tissue and the mesenchymal cord lining stem cells, respectively, are usually cultivated at 37° C. in normal atmosphere with 5% CO2. In this context, it is noted that the mesenchymal cell population as described herein may be derived of any mammalian species, such as human, red deer, mouse, rat, guinea pig, pig, rabbit, goat, horse, dog, cat, sheep, or monkey. In illustrative embodiments, mesenchymal stem cells of the umbilical cord are of red deer or human.

Once a desired/suitable number of mesenchymal stem cells have been obtained from the culture or subculture, the mesenchymal stem cells are harvested by removing them from the cultivation container used for the cultivation. The harvesting of the mesenchymal stem cells is typically again carried out by enzymatic treatment, including comprises trypsination of the cells. The isolated mesenchymal stem cells are subsequently collected and are either be directedly used or preserved for further use. Typically, preserving is carried out by cryo-preservation. The term “cryo-preservation” is used herein in its regular meaning to describe a process where the mesenchymal stem cells are preserved by cooling to low sub-zero temperatures, such as (typically) −80° C. or −196° C. (the boiling point of liquid nitrogen). Cryo-preservation can be carried out as known to the person skilled in the art and can include the use of cryo-protectors such as dimethylsulfoxide (DMSO) or glycerol, which slow down the formation of ice-crystals in the cells of the umbilical cord.

The isolated population of the mesenchymal stem cells that is obtained by the cultivation and/or isolation method as described herein is highly defined and homogenous. Such a highly homogenous population of mesenchymal stem cells derived from tissue such as the amniotic membrane or Wharton's Jelly of the umbilical cord has been reported in WO 2018/067071 A1 and WO 2019/199234 A1. For example, as disclosed and shown in the experimental section in WO 2019/199234 A1A1, at least about 90%, at least about 91% or more, about 92% or more, about 93% or more, about 94% or more, about 95% or more, about 96% or more, about 97% or more, about 98% or more about 99% or more cells of the mesenchymal stem cell population of the umbilical cord (for example, isolated from Wharton's jelly or the amniotic membrane of the umbilical cord) express each of the following markers: CD73, CD90 and CD105 and lack expression of CD34, CD45 and HLA-DR, meaning that this percentage of the isolate cell population express each of CD73, CD90 and CD105 and lack expression of the following markers: CD34, CD45 and HLA-DR. Such a highly homogenous population of mesenchymal stem cells derived from the amniotic membrane of the umbilical cord meets the criteria for mesenchymal stem cells to be used for cellular therapy (also cf. the Experimental Section in WO 2019/199234 A1 and, for example, Sensebe et al. “Production of mesenchymal stromal/stem cells according to good manufacturing practices: a review”, supra). It is noted in this context that this mesenchymal stem cell population can be obtained by either the isolating method as described in WO 2019/199234 A1 but also by a different method such as cell sorting, if wanted.

In a further aspect the invention is directed to a method of inducing, stimulating and/or promoting hair growth and/or hair regeneration, wherein the method comprises treating the hair of a subject with conditioned medium derived from cultivation of mesenchymal stem cells of the umbilical cord in a culture medium as described herein, wherein the culture medium may further comprise mixing to obtain a final volume of 500 ml culture medium:
i. 250 ml of DMEM
ii. 118 ml M171
iii. 118 ml DMEM/F12
iv. 12.5 ml Fetal Bovine Serum (FBS) to reach a final concentration of 2.5% (v/v).

As explained above, DMEM/F12 medium is a 1:1 mixture of DMEM and Ham's F12 medium. Thus, 118 ml DMEM/F12 medium contain 59 ml DMEM and 59 ml F12. Accordingly, when using this method of making a culture medium, the final concentrations (v/v) with 500 ml total volume are as follows:
DMEM: 250 ml+59 ml=309 ml, corresponds to 309/500=61.8% (v/v)
M171: 118 ml, corresponds to 118/500=23.6% (v/v)
F12: 59 ml, corresponds to 59/500=11.8% (v/v).

The culture medium as described herein may further comprise adding
v. 1 ml EGF stock solution (5 μg/ml) to achieve a final EGF concentration of 10 ng/ml, and
vi. Insulin 0.175 ml stock solution (14.28 mg/ml) to achieve a final insulin concentration of 5 μg/ml.

It is noted here that the above-mentioned volumes of these components i. to vi when mixed result in a final volume of 499.675 ml culture medium. If no further components are added to the culture medium, the remaining 0.325 ml (to add up to a volume of 500 ml) can, for example, be any of components i. to iv, that means either DMEM, M171, DMEM/F12 or FBS. Alternatively, the concentration of the stock solution of EGF or Insulin can of course be adjusted such that the total volume of the culture medium is 500 ml. In addition, it is also noted that components i. to iv. do not necessarily have to be added in the order in which they are listed but it is of course also possible to use any order to mix these components to arrive at the culture medium as described herein. This means, that for example, M171 and DMEM/F12 can be mixed together and then combined with DMEM and FBS to reach final concentrations as described here, i.e., a final concentration of DMEM of about 55 to 65% (v/v), a final concentration of F12 of about 5 to 15% (v/v), a final concentration of M171 of about 15 to 30% (v/v) and a final concentration of FBS of about 1 to 8% (v/v).

In other embodiments, the method further comprises adding to DMEM a volume of 0.325 ml of one or more of the following supplements: adenine, hydrocortisone, 3,3′,5-Triiodo-L-thyronine sodium salt (T3), thereby reaching a total volume of 500 ml culture medium. In this embodiment, the final concentration of these supplements in DMEM may be as follows:
about 0.05 to 0.1 μg/ml adenine, for example about 0.025 μg/ml adenine,
about 1 to 10 μg/ml hydrocortisone,
about 0.5 to 5 ng/ml 3,3′,5-Triiodo-L-thyronine sodium salt (T3), for example 1.36 ng/ml 3,3′,5-Triiodo-L-thyronine sodium salt (T3).

In some embodiments, conditioned medium of the present invention is produced by culturing mesenchymal stem cells of the umbilical cord in a cell culture medium as described herein, such as in a culture medium comprising DMEM (Dulbecco's modified eagle medium), F12 (Ham's F12 Medium), M171 (Medium 171) and FBS (Fetal Bovine Serum), removing the mesenchymal stem cells of the umbilical cord from the culture medium, wherein the conditioned medium is obtained by collecting the cell culture medium. Additionally, in some examples, the culture medium in which the stem cells have been cultivated may be replaced by and the cells may be cultivated in a second cell culture medium, which may be the same or a different cell culture medium than the first one. In some examples, the second cell culture medium may be a growth factor-free medium and/or a serum-free medium. Alternatively, mesenchymal stem cells of the umbilical cord that have been previously cultured (e.g., cultured stem cells that have been stored) can be further cultivated in growth factor-free and/or serum-free medium. In either case, the resultant medium is referred to as conditioned medium. The resultant conditioned medium is harvested, and may be further processed to produce a composition comprising conditioned medium of the present invention.

In some embodiments, conditioned medium of the present invention or a composition comprising the conditioned medium of the present invention is diluted in carrier medium and has a final concentration of, for example, about 10% to about 80% (v/v) or for example, a final concentration of about 25% to about 50% (v/v). Conditioned medium of the present invention or a composition comprising the conditioned medium of the present invention can be diluted in any suitable diluent/carrier medium as long as the carrier medium does not affect function and safety of the conditioned medium when topically applied on the skin/scalp of a subject. For example, conditioned medium may be diluted in PBS, water, a basal medium or a cream formulation. A basal medium as used herein refers to a mixture containing saccharides, amino acids, water, etc., required for the survival of a cell and include commercial prepared medium such as but not limited to Dulbecco's Modified Eagle's Medium (DMEM), Endothelial differentiation medium (EDM), Minimal Essential Medium (MEM), Basal Medium Eagle (BME), RPMI 1640, F-10, F-12, α-Minimal Essential Medium (α-MEM), Glasgow's Minimal Essential Medium (G-MEM), and Iscove's Modified Dulbecco's Medium. Additionally, the conditioned medium to be used may be any basic culture medium suitable for animal cell growth, and non-limiting examples of the medium may include Minimal Essential Medium (MEM), Dulbecco modified Eagle Medium (DMEM), Roswell Park Memorial Institute Medium (RPMI), and Keratinocyte Medium (KM), KBM (Keratinocyte Basal medium), EpiLife KM (Keratinocyte-EpiLife medium) etc. In some examples, conditioned medium comprises a cell culture medium as described herein, such as a culture medium comprising DMEM (Dulbecco's modified eagle medium), F12 (Ham's F12 Medium), M171 (Medium 171) and FBS (Fetal Bovine Serum).

In accordance with the above, conditioned medium derived from cultivation of mesenchymal stem cells of the umbilical cord of the present invention can also be comprised in a composition. Conditioned medium or a composition comprising the conditioned medium as described herein and in accordance of the present invention can be applied to the scalp, face and other areas of the skin in which hair growth can occur, particularly at the hair line. As used herein, the hair line is to be understood as the base of the hair on the surface of the skin of a subject and is not restricted to the edge of a subject's hair such as along the top of the forehead but also includes the base of the hair at any surface of the skin of a subject, particularly the scalp. The area for applying conditioned medium or a composition comprising the conditioned medium as described herein and in accordance of the present invention includes not only the scalp but any part of the skin of the body that requires hair-growth. The skin to be treated by conditioned medium of the present invention can be any skin area where hair growth is possible and, for example, can be an area where hairs or other body hairs are damaged, e.g., due to a scar caused by an injury, or areas which require an effect of beauty care such as a wide forehead or M-type forehead, eyelashes or eyebrows. Thus, the skilled person knows and is able to identify a skin area which is capable to grow hair and which is therefore suitable for a method of inducing, stimulating and/or promoting hair growth and/or hair regeneration by using conditioned medium of the present invention.

In some embodiments, the composition as described herein is a pharmaceutical or cosmetic composition for inhibiting hair loss and enhancing hair growth. The composition may be prepared in any formulation conventionally manufactured in the art, and usually comprises conditioned medium of the present invention together with a cosmetically or pharmaceutically suitable acceptable carrier/recipient. The compositions of the invention may also contain additives and adjuvants which are conventional in the cosmetic, pharmaceutical or dermatological field, such as hydrophilic or lipophilic gelling agents, hydrophilic or lipophilic active agents, preservatives, antioxidants, solvents, fragrances, fillers, bactericides, odor absorbers and dyestuffs or colorants. Typically, the composition may be used via dermal application by directly applying it on the skin, particularly on the scalp, or hair such as a hair transplant prior to hair transplantation. Preferably, a composition comprising conditioned medium of the present invention is a cosmetic composition. In some embodiments, the composition is adapted for topical application. A composition adapted for topical application may be in liquid or viscous form. Examples thereof include an ointment, a cream, and a lotion and the like. The preparation of such compositions is within the knowledge of the person skilled in the art and described in Gennaro, A. L. and Gennaro, A. R. (2000) Remington: The Science and Practice of Pharmacy, 20th Ed., Lippincott Williams & Wilkins, Philadelphia, PA, for example. In this context, a composition comprising conditioned medium of the present invention for topical application is to be understood as a formulation that is able to be absorbed into the hair follicle structures in a targeted manner, particularly wherein the active ingredients of the conditioned medium of the present invention or the composition comprising the conditioned medium of the present invention are able to penetrate the layers of the epidermis and dermis sufficiently to reach the hair follicles and the hair follicle bulb matrices, but not to be absorbed into the systemic circulation (see the Example Section in this regard). In some examples, the conditioned medium of the present invention or the composition comprising the conditioned medium of the present invention crosses the surface of the skin and travels to the hair follicles by trans-epidermal diffusion or by transdermal diffusion. In some examples, the conditioned medium of the present invention is injected superficially under the scalp without being absorbed into the systemic circulation. The method of applying conditioned medium or a composition comprising conditioned medium of the present invention may include any method disclosed in the art and the frequency of its application may vary according to the skin condition. Additionally, the skin of a subject may be treated by microneedles prior to the application of conditioned medium or a composition comprising conditioned medium of the present invention. Any skin-penetrating component may be used prior to the application of conditioned medium or a composition comprising conditioned medium of the present invention. As shown in the experimental section, the skin of a subject can be treated with a Dermapen which is a micro-needling device for penetrating the skin using microneedles of 1-3 mm in length.

As another example, conditioned medium derived from cultivation of mesenchymal stem cells of the umbilical cord of the present invention can be used for preparation of a hair transplant. As is generally known to the person skilled in the art, conventional hair transplantation procedures involve removing hair from a region of a patient's scalp which contains permanently-growing hair, and implanting the hair into a balding region of the patient's scalp. In some examples of the present invention, conditioned medium or composition comprising conditioned medium of the present invention for treating hair loss/hair thinning can be for use in preparing of a hair transplant before transplanting hair into a bald area of a patient for treating hair loss/hair thinning, wherein hair has been removed from a subject and incubated in conditioned medium of the present invention until hair follicle growth is induced before implanting the hair into a balding region of the patient's skin such as the scalp.

Accordingly, a method of treating a subject with conditioned medium derived from cultivation of mesenchymal stem cells of the umbilical cord is described. This method comprises administering to the hair of the subject or isolated hair for preparation of a hair transplant an effective amount of the conditioned medium of the present invention. Equally, the present invention relates to conditioned medium and a composition comprising the conditioned medium for use in inducing, stimulating and/or promoting hair growth and/or hair regeneration. Alternatively, or additionally, the present invention relates to conditioned medium and a composition comprising the conditioned medium for use in alleviating and/or reducing hair loss and/or hair thinning. According to the present invention, the use comprises treating the hair of a subject with conditioned medium derived from cultivation of mesenchymal stem cells of the umbilical cord. As illustrated in the Example Section (see Examples 6 to 8, for instance), an “effective amount of the conditioned medium” may be an amount such as 1 ml, 2.5 ml or 5 ml of conditioned medium (either undiluted or diluted to a concentration of, for example, 50% (v/v) in a carrier medium) that is applied per treatment to the skin/scalp of a subject. A treatment course can include repeated administration of such an effective amount over a period of time as described here.

In principle, any subject is suitable for being treated by means of conditioned medium of the present invention. For example, the subject to be treated can suffer from hair loss and/or hair thinning. Finally, the invention also provides method of treating a non-human mammal or a human subject, particularly a subject or a patient suffering from hair loss and/or hair thinning, the method comprising administering to the hair of the subject an effective amount of conditioned medium or a composition comprising conditioned medium of the present invention.

The hair loss and/or hair thinning can be male or female hair loss and can be caused any disease or conditioned, in particular any disease or condition associated with hair loss/hair thinning and in which re-growing of hair is wanted/required. The hair loss may be hormone-related hair loss such as menopausal related hair loss or postpartum related hair loss. The subject may suffer from hair loss that is caused by a disease including inflammatory-induced diseases such as alopecia such as androgenetic alopecia, or alopecia areata; or telogen effluvium such as virally inducted telogen effluvium, including, for example, COVID-19 induced telogen effluvium or dengue virus induced telogen effluvium. Examples of alopecia areata that can be treated with the conditioned medium as described here include alopecia areata monolocularis, alopecia areata multilocularis, ophiasis, alopecia totalis or alopecia universalis. The hair loss may also be caused by a condition such as hair loss due to systemic medical problems, e.g., thyroid disease, adverse drug effects or effects of drug treatment such as cancer chemotherapy, and nutritional deficiency states. In this regard, the hair loss may be chronic or acute. In some examples, the hair loss may be chronic. In some embodiments, the subject suffers from COVID-19 induced hair loss, particularly hair loss that has commenced after an infection of COVID-19. For example, as described in the Experimental Section herein, the patient suffers from COVID-19 induced telogen effluvium (TE) (which refers to diffuse scalp alopecia). In some other embodiments, the subject suffers from androgenic alopecia (AGA).

The invention will be further illustrated by the following non-limiting Experimental Examples.

Sequences as used herein are depicted in below Table 1.

TABLE 1
Sequences as used herein.

| SEQ ID | | |
|---|---|---|
| NO. | What | Sequence |
| 1 | ANGP1 | MTVFLSFAFLAAILTHIGCSNQRRSPENSGRRYNRIQHGQCAYT |
| | (ANG-1) | FILPEHDGNCRESTTDQYNTNALQRDAPHVEPDFSSQKLQHLE |
| | human | HVMENYTQWLQKLENYIVENMKSEMAQIQQNAVQNHTATMLEI |
| | Uniprot no: | GTSLLSQTAEQTRKLTDVETQVLNQTSRLEIQLLENSLSTYKLE |
| | Q15389 | KQLLQQTNEILKIHEKNSLLEHKILEMEGKHKEELDTLKEEKENL |
| | of Apr. 3, | QGLVTRQTYIIQELEKQLNRATTNNSVLQKQQLELMDTVHNLVN |
| | 2013 | LCTKEGVLLKGGKREEEKPFRDCADVYQAGFNKSGIYTIYINNM |
| | | PEPKKVFCNMDVNGGGWTVIQHREDGSLDFQRGWKEYKMGF |
| | | GNPSGEYWLGNEFIFAITSQRQYMLRIELMDWEGNRAYSQYD |
| | | RFHIGNEKQNYRLYLKGHTGTAGKQSSLILHGADFSTKDADND |
| | | NCMCKCALMLTGGWWFDACGPSNLNGMFYTAGQNHGKLNGI |
| | | KWHYFKGPSYSLRSTTMMIRPLDF |
| 2 | HGF | MWVTKLLPALLLQHVLLHLLLLPIAIPYAEGQRKRRNTIHEFKKS |
| | human | AKTTLIKIDPALKIKTKKVNTADQCANRCTRNKGLPFTCKAFVFD |
| | Uniprot no: | KARKQCLWFPFNSMSSGVKKEFGHEFDLYENKDYIRNCIIGKG |
| | P14210 | RSYKGTVSITKSGIKCQPWSSMIPHEHSFLPSSYRGKDLQENY |
| | of Dec. | CRNPRGEEGGPWCFTSNPEVRYEVCDIPQCSEVECMTONGE |
| | 16, 2008 | SYRGLMDHTESGKICQRWDHQTPHRHKFLPERYPDKGFDDNY |
| | | CRNPDGQPRPWCYTLDPHTRWEYCAIKTCADNTMNDTDVPLE |
| | | TTECIQGQGEGYRGTVNTIWNGIPCQRWDSQYPHEHDMTPEN |
| | | FKCKDLRENYCRNPDGSESPWCFTTDPNIRVGYCSQIPNCDM |
| | | SHGQDCYRGNGKNYMGNLSQTRSGLTCSMWDKNMEDLHRHI |
| | | FWEPDASKLNENYCRNPDDDAHGPWCYTGNPLIPWDYCPISR |
| | | CEGDTTPTIVNLDHPVISCAKTKQLRVVNGIPTRTNIGWMVSLR |
| | | YRNKHICGGSLIKESWVLTARQCFPSRDLKDYEAWLGIHDVHG |
| | | RGDEKCKQVLNVSQLVYGPEGSDLVLMKLARPAVLDDFVSTID |
| | | LPNYGCTIPEKTSCSVYGWGYTGLINYDGLLRVAHLYIMGNEKC |
| | | SQHHRGKVTLNESEICAGAEKIGSGPCEGDYGGPLVCEQHKM |
| | | RMVLGVIVPGRGCAIPNRPGIFVRVAYYAKWIHKIILTYKVPQS |
| 3 | VEGFA | MNFLLSWVHWSLALLLYLHHAKWSQAAPMAEGGGQNHHEVV |
| | human | KFMDVYQRSYCHPIETLVDIFQEYPDEIEYIFKPSCVPLMRCGG |
| | Uniprot no: | CCNDEGLECVPTEESNITMQIMRIKPHQGQHIGEMSFLQHNKC |
| | P15692 | ECRPKKDRARQEKKSVRGKGKGQKRKRKKSRYKSWSVYVGA |
| | of Apr. 16, | RCCLMPWSLPGPHPCGPCSERRKHLFVQDPQTCKCSCKNTD |
| | 2014 | SRCKARQLELNERTCRCDKPRR |
| 4 | VEGFB | MSPLLRRLLLAALLQLAPAQAPVSQPDAPGHQRKVVSWIDVYT |
| | Human | RATCQPREVVVPLTVELMGTVAKQLVPSCVTVQRCGGCCPDD |
| | Uniprot no: | GLECVPTGQHQVRMQILMIRYPSSQLGEMSLEEHSQCECRPK |
| | P49765 | KKDSAVKPDRAATPHHRPQPRSVPGWDSAPGAPSPADITHPT |
| | of Nov. | PAPGPSAHAAPSTTSALTPGPAAAAADAAASSVAKGGA |
| | 16, 2001 | |
| 5 | TGFB1 | MPPSGLRLLLLLLPLLWLLVLTPGRPAAGLSTCKTIDMELVKRK |
| | (TGF-β1) | RIEAIRGQILSKLRLASPPSQGEVPPGPLPEAVLALYNSTRDRVA |
| | human | GESAEPEPEPEADYYAKEVTRVLMVETHNEIYDKFKQSTHSIYM |
| | Uniprot no: | FFNTSELREAVPEPVLLSRAELRLLRLKLKVEQHVELYQKYSNN |
| | P01137 | SWRYLSNRLLAPSDSPEWLSFDVTGVVRQWLSRGGEIEGFRL |
| | of Oct. | SAHCSCDSRDNTLQVDINGFTTGRRGDLATIHGMNRPFLLLMA |
| | 13, 2009 | TPLERAQHLQSSRHRRALDTNYCFSSTEKNCCVRQLYIDFRKD |
| | | LGWKWIHEPKGYHANFCLGPCPYIWSLDTQYSKVLALYNQHNP |
| | | GASAAPCCVPQALEPLPIVYYVGRKPKVEQLSNMIVRSCKCS |
| 6 | PDGFA | MRTLACLLLLGCGYLAHVLAEEAEIPREVIERLARSQIHSIRDLQ |
| | (PDGF-AA) | RLLEIDSVGSEDSLDTSLRAHGVHATKHVPEKRPLPIRRKRSIEE |
| | human | AVPAVCKTRTVIYEIPRSQVDPTSANFLIWPPCVEVKRCTGCCN |
| | Uniprot no: | TSSVKCQPSRVHHRSVKVAKVEYVRKKPKLKEVQVRLEEHLEC |
| | P04085, | ACATTSLNPDYREEDTGRPRESGKKRKRKRLKPT |
| | of Feb. | |
| | 10, 2009 | |
| 7 | PDGFB | MNRCWALFLSLCCYLRLVSAEGDPIPEELYEMLSDHSIRSFDDL |
| | human | QRLLHGDPGEEDGAELDLNMTRSHSGGELESLARGRRSLGSL |
| | Uniprot no: | TIAEPAMIAECKTRTEVFEISRRLIDRTNANFLVWPPCVEVQRCS |
| | P01127 | GCCNNRNVQCRPTQVQLRPVQVRKIEIVRKKPIFKKATVTLEDH |
| | of Feb. | LACKCETVAAARPVTRSPGGSQEQRAKTPQTRVTIRTVRVRRP |
| | 06, 2013 | PKGKHRKFKHTHDKTALKETLGA |
| 8 | FGF2 (bFGF) | MGDRGRGRALPGGRLGGRGRGRAPERVGGRGRGRGTAAPR |
| | human | AAPAARGSRPGPAGTMAAGSITTLPALPEDGGSGAFPPGHFKD |
| | Uniprot no: | PKRLYCKNGGFFLRIHPDGRVDGVREKSDPHIKLQLQAEERGV |
| | P09038 Jul. | VSIKGVCANRYLAMKEDGRLLASKCVTDECFFFERLESNNYNT |
| | 28, 2009 | YRSRKYTSWYVALKRTGQYKLGSKTGPGQKAILFLPMSAKS |
| 9 | CCL2 | MKVSAALLCLLLIAATFIPQGLAQPDAINAPVTCCYNFTNRKISV |
| | (MCP-1) | QRLASYRRITSSKCPKEAVIFKTIVAKEICADPKQKWVQDSMDH |
| | human | LDKQTQTPKT |
| | Uniprot no: | |
| | P13500 | |
| | Oct. 13, | |
| | 2009 | |
| 10 | IL6 | MNSFSTSAFGPVAFSLGLLLVLPAAFPAPVPPGEDSKDVAAPH |
| | (IL-6) | RQPLTSSERIDKQIRYILDGISALRKETCNKSNMCESSKEALAEN |
| | human | NLNLPKMAEKDGCFQSGFNEETCLVKIITGLLEFEVYLEYLQNR |
| | Uniprot no: | FESSEEQARAVQMSTKVLIQFLQKKAKNLDAITTPDPTTNASLLT |
| | P05231 | KLQAQNQWLQDMTTHLILRSFKEFLQSSLRALRQM |
| | of Jan. | |
| | 10, 2003 | |
| 11 | IL8 | MTSKLAVALLAAFLISAALCEGAVLPRSAKELRCQCIKTYSKPFH |
| | (IL-8) | PKFIKELRVIESGPHCANTEIIVKLSDGRELCLDPKENWVQRVVE |
| | human | KFLKRAENS |
| | Uniprot no: | |
| | P10145 | |
| | of May 5, | |
| | 2009 | |
| 12 | TNR1A (TNF | MGLSTVPDLLLPLVLLELLVGIYPSGVIGLVPHLGDREKRDSVCP |
| | receptor 1) | QGKYIHPQNNSICCTKCHKGTYLYNDCPGPGQDTDCRECESG |
| | human | SFTASENHLRHCLSCSKCRKEMGQVEISSCTVDRDTVCGCRK |
| | Uniprot no: | NQYRHYWSENLFQCFNCSLCLNGTVHLSCQEKQNTVCTCHAG |
| | P19438 | FFLRENECVSCSNCKKSLECTKLCLPQIENVKGTEDSGTTVLLP |
| | of Jul. 24, | LVIFFGLCLLSLLFIGLMYRYQRWKSKLYSIVCGKSTPEKEGELE |
| | 2013 | GTTTKPLAPNPSFSPTPGFTPTLGFSPVPSSTFTSSSTYTPGDC |
| | | PNFAAPRREVAPPYQGADPILATALASDPIPNPLQKWEDSAHK |
| | | PQSLDTDDPATLYAVVENVPPLRWKEFVRRLGLSDHEIDRLEL |
| | | QNGRCLREAQYSMLATWRRRTPRREATLELLGRVLRDMDLLG |
| | | CLEDIEEALCGPAALPPAPSLLR |
| 13 | TIMP1 | MAPFEPLASGILLLLWLIAPSRACTCVPPHPQTAFCNSDLVIRAK |
| | human | FVGTPEVNQTTLYQRYEIKMTKMYKGFQALGDAADIRFVYTPA |
| | Uniprot no: | MESVCGYFHRSHNRSEEFLIAGKLQDGLLHITTCSFVAPWNSL |
| | P01033 | SLAQRRGFTKTYTVGCEECTVFPCLSIPCKLQSGTHCLWTDQL |
| | of Jun. 20, | LQGSEKGFQSRHLACLPREPGLCTWQSLRSQIA |
| | 2002 | |
| 14 | TR10C | MARIPKTLKFVVVIVAVLLPVLAYSATTARQEEVPQQTVAPQQQ |
| | human | RHSFKGEECPAGSHRSEHTGACNPCTEGVDYTNASNNEPSCF |
| | Uniprot no: | PCTVCKSDQKHKSSCTMTRDTVCQCKEGTFRNENSPEMCRK |
| | 014798 of | CSRCPSGEVQVSNCTSWDDIQCVEEFGANATVETPAAEETMN |
| | Sept. | TSPGTPAPAAEETMNTSPGTPAPAAEETMTTSPGTPAPAAEET |
| | 23, 2008 | MTTSPGTPAPAAEETMTTSPGTPASSHYLSCTIVGIIVLIVLLIVF |
| | | V |
| 15 | UPAR | MGHPPLLPLLLLLHTCVPASWGLRCMQCKTNGDCRVEECALG |
| | human | QDLCRTTIVRLWEEGEELELVEKSCTHSEKTNRTLSYRTGLKIT |
| | Uniprot no: | SLTEVVCGLDLCNQGNSGRAVTYSRSRYLECISCGSSDMSCE |
| | Q03405 | RGRHQSLQCRSPEEQCLDVVTHWIQEGEEGRPKDDRHLRGC |
| | version | GYLPGCPGSNGFHNNDTFHFLKCCNTTKCNEGPILELENLPQN |
| | number 1 as | GRQCYSCKGNSTHGCSSEETFLIDCRGPMNQCLVATGTHEPK |
| | of May 5, | NQSYMVRGCATASMCQHAHLGDAFSMNHIDVSCCTKSGCNH |
| | 2009 | PDLDVQYRSGAAPQPGPAHLSLTITLLMTARLWGGTLLWT |
| 16 | ICAM1 | MAPSSPRPALPALLVLLGALFPGPGNAQTSVSPSKVILPRGGSV |
| | human | LVTCSTSCDQPKLLGIETPLPKKELLLPGNNRKVYELSNVQEDS |
| | Uniprot no: | QPMCYSNCPDGQSTAKTFLTVYWTPERVELAPLPSWQPVGKN |
| | P05362 | LTLRCQVEGGAPRANLTVVLLRGEKELKREPAVGEPAEVTTTV |
| | of Oct. | LVRRDHHGANFSCRTELDLRPQGLELFENTSAPYQLQTFVLPA |
| | 13, 2009 | TPPQLVSPRVLEVDTQGTVVCSLDGLFPVSEAQVHLALGDQRL |
| | | NPTVTYGNDSFSAKASVSVTAEDEGTQRLTCAVILGNQSQETL |
| | | QTVTIYSFPAPNVILTKPEVSEGTEVTVKCEAHPRAKVTLNGVP |
| | | AQPLGPRAQLLLKATPEDNGRSFSCSATLEVAGQLIHKNQTRE |
| | | LRVLYGPRLDERDCPGNWTWPENSQQTPMCQAWGNPLPELK |
| | | CLKDGTFPLPIGESVTVTRDLEGTYLCRARSTQGEVTRKVTVVL |
| | | SPRYEIVIITVVAAAVIMGTAGLSTYLYNRQRKIKKYRLQQAQKG |
| | | TPMKPNTQATPP |
| 17 | IGF1 | MGKISSLPTQLFKCCFCDFLKVKMHTMSSSHLFYLALCLLTFTS |
| | human | SATAGPETLCGAELVDALQFVCGDRGFYFNKPTGYGSSSRRA |
| | Uniprot no: | PQTGIVDECCFRSCDLRRLEMYCAPLKPAKSARSVRAQRHTD |
| | P05019 | MPKTQKYQPPSTNKNTKSQRRKGWPKTHPGGEQKEGTEASL |
| | of Jun. 26, | QIRGKKKEQRREIGSRNAECRGKKGK |
| | 2013 | |

### EXPERIMENTAL EXAMPLES

### Example 1: Isolation and Cultivation of Mesenchymal Stem Cells of the Umbilical Cord (CLMSCs)

### 1. Preparation of Medium for Processing CLMSCs

a. To make 500 ml PTT-6 (culture/growth medium) add the following in the order listed:
i. DMEM, 250 ml
ii. M171 118 ml
iii. DMEMF12 118 ml
iv. FBS 12.5 ml (final concentration of 2.5%)
v. EGF 1 ml (final concentration of 10 ng/ml)
vi. Insulin 0.175 ml (final concentration of 5 μg/ml)

The above-mentioned volumes of components i. to vi when result in a final volume of 499.675 ml culture medium. If no further components are added to the culture medium, the remaining 0.325 ml (to add up to a volume of 500 ml) can, for example, be any of components i. to iv, that means either DMEM, M171, DMEM/F12 or FBS. Alternatively, the concentration of the stock solution of EGF or Insulin can of course be adjusted such that the total volume of the culture medium is 500 ml. Alternatively, a stock solution of an antibiotic such as Penicillin-Streptomycin-Amphotericin can be added to result in a final volume of 500 ml. It is also possible to add to the culture medium a volume of 0.325 ml of one or more of the following supplements: adenine, hydrocortisone, 3,3′,5-Triiodo-Fthyronine sodium salt (T3), thereby reaching a total volume of 500 ml culture medium.

vii. Label the bottle “PTT-6” with date medium was prepared, initial of the operator, and the phrase “expires on” followed by the expiration date. Expiration date is the earliest expiration date of any of the component or 1 month from the preparation date, whichever comes first.

b. To make the rinse medium (Hank's Buffered Salt Solution (HBSS) without Calcium or Magnesium and with 5% FBS), add 2.5 ml FBS to 47.5 ml of HBSS in a 50 ml centrifuge tube. Label the tube “Rinse Medium” with operator initials and date the medium is made.

c. All medium will be tested for sterility using Bactec Fytic/10 Dickinson & Company) and Bactec Pluc+Aerobic/F (Becton Dickinson & Company). Inject 20 ml of prepared medium into each bottle.

### 2. Collection and Isolation of Human and Red Deer (RD) Umbilical Cord Tissue

Red deer (RD) umbilical cords were collected under specific handling instructions from a farm in New Zealand that rears the animal for collection of horn velvet. RD-CLMSC were isolated from umbilical cords according to the protocol described in International Patent Application WO 2006/019357 A1. Human Umbilical cord tissue (the umbilical cords were donated with informed consent of the mother) was processed for the subsequent isolation of the mesenchymal stem cells from the umbilical cord as described in International Patent Application or WO2018/067071 A1 or WO 2019/199234 A1.

In brief, the umbilical cords of red deer and human are rinsed clean and immediately transferred into a 500 ml sterile glass bottle containing culture transport medium (L-15 medium supplemented with 50 IU/ml penicillin, 50 μg/ml streptomycin, 250 μg/ml fungizone, 50 μg/ml gentamicin; all reagents purchased from Invitrogen) prior to transport to the laboratory. In the laboratory, stem cell extraction is conducted in a laminar flow hood under sterile conditions. The umbilical cords are first transferred to a sterile stainless steel tray. All remaining blood in the cord vessels is removed by multiple syringing washes using warm phosphate-buffered saline (PBS) supplemented with 5 IU/ml heparin (from Sigma). Plain PBS without heparin is used in the final washes. The umbilical cord tissue specimen is then cut into pieces 2 cm in length and transferred into 10 cm diameter cell culture dishes, where further washing and disinfection is performed with 70% ethanol followed by multiple washes using PBS containing an antibiotic mixture (50 IU/ml penicillin, 50 μg/ml streptomycin, 250 μg/ml fungizone, 50 μg/ml gentamicin; all purchased from Invitrogen) until the solution becomes clear.

### 3. Isolation and Cultivation of RD-CLMSC and H-CLMSC

Dissection of umbilical cord tissue is first performed to separate the umbilical cord amniotic membrane from Wharton's jelly (i.e. the matrix of umbilical cord) and other internal components. The isolated amniotic membrane is then cut into small pieces (0.5 cm×0.5 cm) for cell isolation. Explant is performed by placing the pieces of umbilical cord amniotic membrane on tissue culture dishes at different cell culture conditions for isolation of mesenchymal stem cells.

For mesenchymal cell isolation/cultivation, the explants were submerged in PTT-6 medium (CellResearch Corp, Singapore) and maintained in a CO₂ cell culture incubator at 37° C. The medium was changed every 2 or 3 days. Cell outgrowth was monitored under light microscopy. At a confluence of about 70%, cells were trypsinized (0.05% trypsin/0.02% EDTA) for further expansion or for cryo-preservation. H-CLMSC (#CLMC43) was also obtained from the CRC tissue bank.

### Example 2: Conditioned Medium Derived from Red Deer Cord Lining Mesenchymal Stem Cells (RD-CLMSCs), Human Cord Lining Mesenchymal Stem Cells (H-CLMSCs) and Human Foreskin Fibroblasts (FSFs)

Cryovials containing RD-CLMSC, H-CLMSC and Human Foreskin Fibroblast (FSF) as control were retrieved from storage and quick thawed in a 37° C. water bath. FSF was given to CRC (CellResearch Corporation, Singapore) as a gift from the Stem Cell and Wound Healing Research Group, Department of Surgery, Yong Loo Lin School of Medicine, National University of Singapore.

PTT-6 medium (CellResearch Corporation, Singapore) was used for culturing RD-CLMSCs and H-CLMSCs at 37° C. with 5% CO₂. DMEM with 10% FCS was used for culturing FSFs at 37° C. with 5% CO₂. The cell culture medium was changed every 2 to 3 days. At 80% to 100% confluency, the cell culture medium was removed and cells may optionally be rinsed in PBS before cultured in DMEM basal medium, wherein the medium optionally comprises a water-soluble antioxidant but no growth factors or serum. Culture dishes were incubated for further 48 hours and spent medium was collected into centrifuge tubes for centrifugation at 1800 rpm for 10 minutes. The supernatant was collected as conditioned medium (CM) into labelled tubes: Red Deer Cord Lining Mesenchymal Stem Cell Conditioned Medium (RD-CLMSC-CM), Human Cord Lining Mesenchymal Stem Cell Conditioned Medium (H-CLMSC-CM) and Human Foreskin Fibroblast Conditioned Medium (FSF-CM). Conditioned media were kept at −80° C. until use.

### Example 3: Expression of Elastin and Hyaluronic Acid (HA) of Human Dermal Fibroblasts (HDF) Cultured in Conditioned Medium of RD-CLMSC and FSF Vs. DMEM/10% FCS

The effect of conditioned medium derived from cultivation of the mesenchymal stem cells of the umbilical cord from red deer (RD-CLMSC) and Human Foreskin Fibroblast (FSF) on human skin was assessed in order to elucidate potential mechanisms of action of conditioned medium from different sources (RD-CLMSC and FSF) on human skin and whether such mechanisms of action are associated with promoting hair growth. For assessing the effect of conditioned medium on human skin, human dermal fibroblasts (HDF) were used and selected from the tissue bank of CellResearch Corporation (CRC) from sources listed in the following table, and were divided into Normal Fibroblasts (NF) for individuals below 60 years of age and Aged Skin Fibroblasts (asF) for individuals 60 years and older.

TABLE 2
HDF and donor profiles

| | Donor Profile | | |
|---|---|---|---|
| | (Male/Female, Ethnicity, | Site of donated | |
| Fibroblasts | Age of donor in years) | skin | Experiments |
| NF 110 | F, Chinese, 23 | Upper eyelid skin | Elastin / HA Expression |
| NF121 | M, Chinese, 54 | Upper eyelid skin | Elastin / HA Expression |
| NF129 | M, Caucasian, 51 | Temporal skin | Elastin / HA Expression |
| NF134 | M, Chinese, 32 | Dorsal forearm skin | Elastin / HA Expression |
| NF139 | F, Chinese, 40 | Forehead skin | Elastin / HA Expression |
| NF142 | F, Chinese, 40 | Upper eyelid skin | Elastin / HA Expression |
| NF153 | F, Malay, 53 | Upper eyelid skin | Elastin / HA Expression |
| NF172 | M, Caucasian, 51 | Upper eyelid skin | Elastin / HA Expression |
| NF214 | F, Caucasian, 44 | Cheek skin | Elastin / HA Expression |
| NF313 | M, Caucasian, 24 | Neck skin | Elastin / HA Expression |
| asF20 | F, Chinese, 69 | Upper eyelid skin | Elastin / HA Expression |
| asF6 | F, Chinese, 62 | Upper eyelid skin | Elastin / HA Expression |
| asF61 | M, Caucasian, 61 | Forehead skin | Elastin / HA Expression |
| asF74 | F, Chinese, 67 | Upper eyelid skin | Elastin / HA Expression |
| | | | Cell Proliferation assay |
| asF75 | F, Chinese, 69 | Upper eyelid skin | Cell Proliferation assay |
| asF76 | M, Caucasian, 73 | Scalp skin | Cell Proliferation assay |

### Method

HDF were seeded in 96-wells plates at a density of 10,000 cells/well in DMEM/10% FCS (LifeTech Holdings, cat.no. 10270106). At approximately 80% confluence, medium was removed and remaining cells in the wells washed once with PBS. Cells were then cultured in DMEM/10% FCS (negative control) or RD-CLMSC-CM for 48 hours followed by immunocytochemical staining for hyaluronic acid (HA) (My BioSource, cat.no. MBS2025717) and elastin (LifeTech Holdings, cat.no. MA 127129); FIG. 1A and FIG. 2A. Briefly, cells were washed with 1×PBS before fixation in cold methanol for 10 mins followed by blocking with 2.5% normal horse serum (Vector Laboratories cat.no.PK-7200) for 20 mins in a humid chamber. Cells were then incubated with primary HA antibodies (1:50) and elastin antibodies (1:50) for 2 hours in room temperature. They were washed with TBS (VWR Life Science cat.no.788) and 1% Tween solution (Sigma Cat.no.P9416) followed by incubation with secondary antibodies (Vector Laboratories cat.no.PK-7200) for 15 mins. Washing was performed a further three times with 1×TBS/1% tween solution before incubation with ABC Reagent (Vector Laboratories cat.no.PK-7200) for 15 mins in room temperature. Finally, DAB (Dako cat.no. K3468) was added for colorimetric development. Harris Haematoxylin (Sigma Cat.No.HHS16) was used for counter staining. Images were taken with a bright field phase contrast microscope (Olympus) at 10× magnification. Expression of Elastin and Hyaluronic Acid were quantified with ImageJ (National Institutes of Health, United States).

### Results

To compare the effects of RD-CLMSC-CM and DMEM/10% FCS (control) on the regulation of elastin and HA expression in HDF, and to assess if these effects were dependent on the age of the donors and the sites from which skin tissue were procured, HDF (n=14) isolated from donors from 23 to 73 years in age and from multiple donor sites (forehead, eyelid, cheek, neck, temple) were selected from a skin library for this set of experiments. The cells were subjected to 48 hours culture in either DMEM/10% FCS or RD-CLMSC-CM before immunocytochemical staining for elastin and HA. Expression levels were quantified as optical densities using ImageJ (National Institutes of Health, United States). As shown in FIGS. 1A and 1B and FIGS. 2A and 2B, RD-CLMSC-CM upregulated the expression of elastin by 56% and HA by 83% in HDF as compared to DMEM/10% FCS. Overall, this set of experiments demonstrates significant upregulation of HDF elastin and hyaluronic acid expression by the conditioned medium of the invention. This effect appeared to be independent of the donor age or the donor site from which the skin was derived.

### Conclusion

Without wishing to being bound by theory, the upregulation of elastin and HA expression present in RD-CLMSC-CM can be postulated to be due to the presence of growth factors including TGF-βs, IGFs, PDGF-BB and FGF-7 present in RD-CLMSC-CM.

### Example 4: Proliferative Effect of Human Dermal Fibroblasts (HDF) in Conditioned Medium Derived from Cultivation of H-CLMSC, RD-CLMSC and FSF Vs. DMEM/10% FCS

### Background

The proliferative properties of Red Deer Cord Lining Mesenchymal Stem Cell Conditioned Medium (RD-CLMSC-CM), Human Cord Lining Mesenchymal Stem Cell Conditioned Medium (H-CLMSC-CM) and Human Foreskin Fibroblast Conditioned Medium (FSF-CM) were investigated and compared to DMEM/10% FCS (control) by exposing aged human dermal fibroblasts (HDF) to these medium over a period of 5 days (FIG. 3). Slow proliferating aged HDF were used in this assay instead of the normal proliferating HDF to amplify the difference in the proliferative effects of the different CMs. Superior proliferative properties suggest the presence of other protein growth factors present in conditioned medium which might be exerting a positive influence on HDF proliferation.

### Method

Aged skin dermal fibroblasts (asF) were isolated from the skin of donors aged 60 years and above (asF74/asF75/asF76, Table 2). asF were seeded equally (in triplicate) into 24 well plates containing DMEM/10% FCS and incubated at 37° C. and 5% CO₂ for 24 hours to allow cellular adherence. After 24 hours, medium was removed from the plates and the wells rinsed once with PBS. At Day 0, HDF were cultured in DMEM/10% FCS (control), FSF-CM, H-CLMSC-CM or RD-CLMSC-CM. Medium was changed on Day 2. On Day 5, medium was removed and the cells were rinsed once with PBS. Cells were trypsinised and observed under the microscope to ensure that all the cells were dislodged before neutralization by the addition of 1 ml DMEM/10% FCS. Cell suspensions were centrifuged at 1800 rpm for 10 mins, supernatants were discarded and the pellet at the bottom of each tube resuspended with 1 ml DMEM. 50 μl of cell suspension was mixed with 50 μl of Trypan Blue (Sigma cat. no. T8154) and incubated for 10 mins. Total cells count was performed using the Countess-II Automated Cell Counter system (Life technologies cat no. AMQAX10001).

### Results

As shown in FIG. 3, in comparison to DMEM/10% FCS (control), the highest increase in average total cell counts was seen in the RD-CLMSC-CM group (113% increase over control), followed by H-CLMSC-CM (112%), and FSF-CM (16%). These results show that the proliferative factors in RD-CLMSC are as efficacious as those in H-CLMSC-CM and demonstrate cross species efficacy without toxic effects on HDF. Without being to be bound by theory, the superior proliferative properties of CLMSC-CM (RD and H) over FSF-CM and the FBS control suggests the presence of other protein growth factors present in the CLMSC-CM which might be exerting a positive influence on HDF proliferation.

### Example 5: Pro-Migrative Properties of RD-CLMSC-CM Compared to FSF-CM Assessed in Ab In-Vitro “Scratch” Wound Assay

### Background

The in vitro “scratch” assay mimics cellular mobility during wound healing and is a well-developed method to measure migration of cells.

### Method

To create a monolayer of cells, each 100-mm dish was seeded with 100,000 HDF and cultured in DMEM/10% FCS at 37° C. and 5% CO₂ until 100% confluent. A ‘scratch’ was created by scraping the monolayer in a straight line using a p200 pipette tip. The culture dish was then rinsed once with PBS to remove any loose cells. The cells were immersed in FSF-CM or RD-CLMSC-CM for 5 days with images taken on Day 0 and Day 5 to document the migration of cells from the edge of the gap.

### Results

As shown in FIG. 4, the pro-migrative properties of RD-CLMSC-CM was compared against FSF-CM by measuring regions that remained unfilled by HDFs after 5 days of culturing in the test CMs. On day 5 all “scratches” in the RD-CLMSC-CM treatment group were completely filled while FSF-CM treatment group were only about 80% filled. These results demonstrate the pro-migrative properties of RD-CLMSC-CM are more potent then FSF-CM. The significant increase in the mobility of HDFs cultured in RD-CLMSC-CM can be attributed to the pro-migrative effects of VEGF, TGF-β1 and HGF.

### Example 6: Promoting Hair Induction Ability with Conditioned Medium Derived from Cultivation of RD-MLSCs

### Background

The aim of this study is to assess the capability of conditioned medium derived from cultivation of mesenchymal stem cells of the umbilical cord as shown in Example 2 to stimulate improved hair growth from underactive or dormant hair follicles in males suffering from androgenic alopecia (AGA). As an exemplary conditioned medium of the present invention, CALECIM®, a novel cosmetic product formulated from conditioned medium of the present invention was used, which contains proteins, in particular growth factors, and exosomes, naturally secreted by mesenchymal stem cells of the umbilical cord.

### Methods

Patients with manifestation of hair thinning/hair loss were selected. Patients, who do not return for follow-up review as scheduled; patients who display any adverse reaction to the CALECIM® product; Thyroid disfunction; Deficiencies of vitamins and iron; Hormone imbalances; Smokers; Pregnant and breastfeeding women; Male patients Norwood Scale 5 and above; Female patients Ludwig Scale 3 and above were excluded from the study.

Once patients were selected photographs were taken before the first treatment; see FIG. 6A to FIG. 6G. Treatments were carried out weekly, usually with a total of 6 treatments performed. The second set of photos were taken during and/or on completion of treatments; see FIG. 6A to FIG. 6G.

The protocol for each treatment was as follows:
Scalp disinfection with 0.5% Chlorhexidine.
2.5 ml of CALECIM® Advanced Hair System formulated from conditioned medium derived from cultivation of red deer mesenchymal stem cells of the umbilical cord was applied and needled into the treatment area using a derma stamp with 1 mm, 1.2 mm and 1.5 mm long microneedles.
An additional 2.5 ml was applied following the derma stamp treatment and gently rubbed into the scalp.
Patients were advised not to wash their hair following the treatment for at least 24 hours and optimally 48 hours.

### Results

As shown in FIGS. 6A-6G, all patients had objective improvement in hair density and coverage over the CALECIM® Advanced Hair System treated area as assessed by the treating physician. All patients reported that they felt improvement in hair quality and coverage as well. Patients received 3 and 6 months follow up to review further progress.

### Example 7: Conditioned Medium Derived from Cultivation of RD-MLSCs Stimulates Improved Hair Growth in Patients with Androgenic Alopecia (AGA)

### Background

Androgenic Alopecia (AGA) also known as male and female pattern baldness is the most common cause of hair loss in the world today. It can affect up to 50% of Caucasian males. AGA has a number of causes and is widely agreed to be multi-factorial with hereditary, environmental, dietary & hormonal influences. Effectively, hair follicle growth cycles reduce in time and the hair produced with in these cycles becomes shorter and thinner. Over time, the follicle becomes completely inactive within the scalp. The aim of this study is to assess the capability of the CALECIM® product formulated from conditioned medium derived from cultivation of red deer mesenchymal stem cells of the umbilical cord as shown in Example 2 to stimulate improved hair growth from underactive or dormant hair follicles in males suffering from AGA.

### Methods

A total of 5 male patients with AGA graded 3-4 on the Norwood scale of hair loss were selected. All participants were screened for vitamin deficiencies, iron deficiency and thyroid function. Patients with abnormal results were excluded from the study. Smokers were also excluded in the study. Once patients were selected photographs were taken before the first treatment. Treatments were carried out weekly, with a total of 6 treatments performed. The second set of photos were taken on completion of the 6 weekly treatments; see FIG. 5.

The protocol for each treatment was as follows:
Scalp disinfection with 0.5% Chlorhexidine.
2.5 ml of a composition containing 80% conditioned medium, 20% aqueous hyaluronic acid (CALECIM® Advanced Hair System) formulated from conditioned medium derived from cultivation of red deer mesenchymal stem cells of the umbilical cord was applied and needled into the treatment area using a derma stamp with 2 mm long microneedles.
An additional 2.5 ml was applied following the derma stamp treatment and gently rubbed into the scalp.
Patients were advised not to wash their hair following the treatment for at least 24 hours and optimally 48 hours.

### Results

As shown in FIG. 5, at week 6 all patients had objective improvement in hair density and coverage over the area treated with the composition containing conditioned medium as assessed by the treating physician. All patients reported that they felt improvement in hair quality and coverage as well. Patients received 3 and 6 months follow up to review further progress.

### Example 8: Case Study-Conditioned Medium Derived from Cultivation of RD-MLSCs Stimulates Improved Hair Growth in a Patient with COVID-19-Induced Scalp Alopecia

### Background

COVID-19-induced scalp Alopecia can induce telogen effluvium (TE) which refers to diffuse scalp alopecia. A patient with severe chronic TE with no spontaneous recovery after 6 months of hair loss and no response to conventional treatment of minoxidil and LED light treatment (Hairmax) was treated with a composition comprising conditioned medium derived from cultivation of mesenchymal stem cells of the umbilical cord (CALECIM® Advanced Hair System).

### Method and Results

The scalp of the patient was progressively treated with rubbing with an alcohol swab, then 1 ml of a composition comprising conditioned medium derived from cultivation of mesenchymal stem cells of the umbilical cord was applied and massaged in till dry. The patient was advised to wait until evening to wash his hair. Five treatments of conditioned medium derived from cultivation of mesenchymal stem cells of the umbilical cord spaced a month apart were administered. The patient had progressive improvement in hair density with regrowth of frontal hairline and villus hair in the prefrontal region (FIG. 7). Hair fragility was reversed with no breakage of the hair on combing.

### Example 9: Growth Promotion Effect of Conditioned Medium on Cultured Human Follicle Dermal Papilla Cells (HFDPC)

### Testing Protocol and Procedure

Hair growth is controlled by a unique repetitive cycle comprised of anagen, catagen and telogen phases. Dermal papilla cells (DPCs), a group of specialized fibroblasts within the hair follicle bulb, have an essential function in the control of hair growth not only in the normal hair cycle but also in the pathogenesis of certain conditions, for example in androgenetic alopecia. Therefore, factors affecting the functions of DPCs in hair loss are of great importance from the therapeutic viewpoint. The proliferation of HFDPC is evaluated by measuring their metabolic activities using 2-(2-methoxy-4-nitrophenyl)-3-(4-nitrophenyl)-5-(2,4-disulfophenyl)-2H-tetrazolium (WST-8), which is a highly water-soluble tetrazolium salt. HFDPCs are either treated with different concentrations of Minodixil (0.0125-200 μg/mL; see Table 3, left column, serving as the reference) or conditioned medium of the invention at different dilutions (PC, dilution range from 10-1280 times; see third column of Table 3). The negative control (NC) refers to HFDPCs incubated with basal medium only.

### Analysis Result

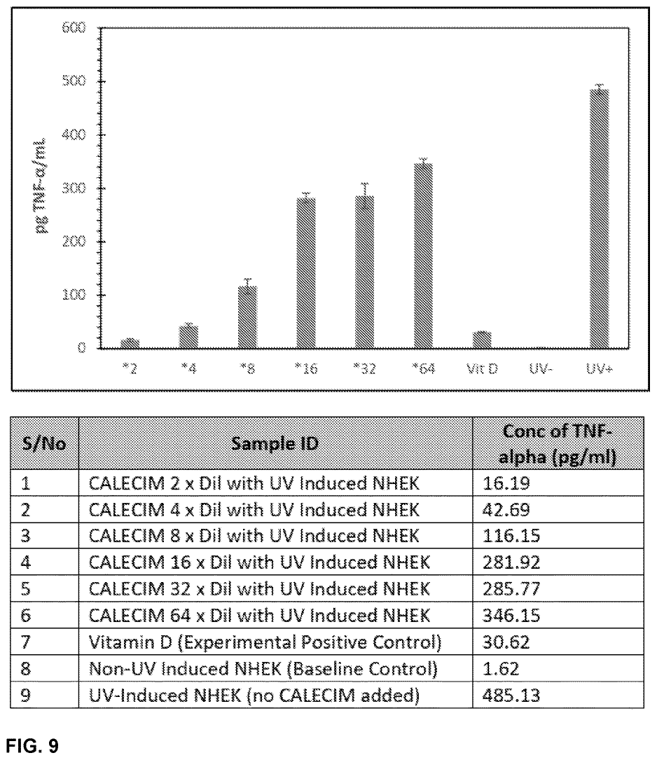

The results are shown in Table 3 and illustrated in FIG. 9. For HFDPC treated with the crude extract, when the concentration is 0.048 mg/mL, there are around 10% more cells in comparison with that of control. For HFDPC treated minoxidil (reference), when diluted 320 times, there are around 20% more cells than that of negative control.

TABLE 3
cell count of HFDPC treated with conditioned medium (right), Minoxidil
(left, as reference compound) and negative control (NC)

| Concentration | | | | Conditioned |
|---|---|---|---|---|
| of | Minoxidil | | NC | medium |
| Minoxidil | (% of | Dilution | (% of | (% of |
| (ug/mL) | control) | factor | control) | control) |
| 200 | 43 ± 2.6 | 10 | 50.6 ± 1.8 | 79.6 ± 6.8 |
| 20 | 98.5 ± 7.7 | 20 | 83.5 ± 8.3 | 105.4 ± 7.5 |
| 2 | 124.5 ± 10.2 | 40 | 93.8 ± 7.3 | 101 ± 5.8 |
| 0.2 | 123.2 ± 8.4 | 80 | 95.3 ± 7.5 | 103.4 ± 7.1 |
| 0.1 | 120.8 ± 8.8 | 160 | 106.1 ± 6 | 111.9 ± 10.2 |
| 0.05 | 120.8 ± 4.8 | 320 | 101.2 ± 9.6 | 124.1 ± 9.5 |
| 0.025 | 92.2 ± 1.4 | 640 | 87 ± 9.2 | 81.1 ± 7.4 |
| 0.0125 | 94 ± 0.6 | 1280 | 75.7 ± 8.8 | ± 2.4 |

### Statistical Analysis

All data are shown as the mean±standard deviation (SD). For comparison of different treatments between 2 groups, the Student's t test was used. p<0.05 was considered significant, p<0.01 would normally be considered highly significant, and p<0.001 extremely significant. After calculation, p is 0.03.

### Conclusion

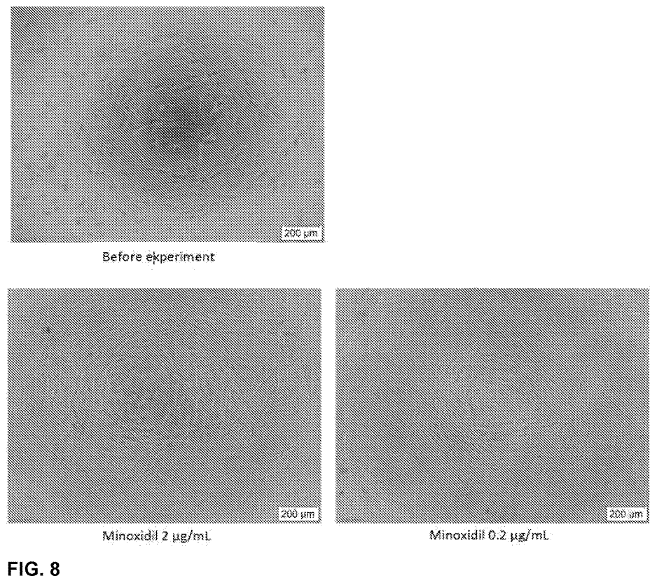

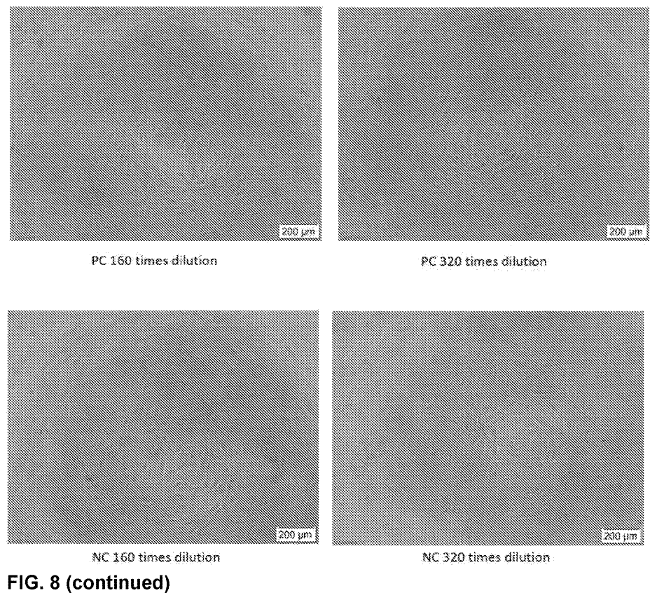

When the sample that was treated with conditioned medium according to the present invention was diluted 160 and 320 times, there are around 10% and 20% more cells compared to negative control-treated cells (see also FIG. 8); this growth promotion effect in this model experiment is similar to the reference compound, minoxidil, for which the optimal concentration is between 0.2 to 2 μg/mL. Or in other word, in this experiment the conditioned medium of the invention has been shown to increase dermal papilla cell proliferation by up to about 24%, comparable to the most effective concentrations of Minoxidil. It is noted here that minoxidil is known to have unwanted and serious side effects including but not limited itching or skin rash, burning of the scalp, inflammation and soreness of the root of the hair, reddened skin, swelling of face and increased hair loss; see, for example, https://www.drugs.com/sfx/rogaine-side-effects.html. To the contrary, no side effects have so far been observed for the conditioned medium of the invention. Further, minoxidil is reported to be effective for treatment of hair loss in only 40% of the patients, while the conditioned medium of the present application has been found to be free of side effects and effective in inducing, stimulating and/or promoting hair growth and hair regeneration in up to 95% of the patients included in the present studies (data not shown). Thus, this example shows that the conditioned medium of the present invention represents a clear improvement over minoxidil.

### Example 10: Anti-Inflammatory Activity of Conditioned Medium (CALECIM) on UV Induced Primary Normal Human Epidermal Keratinocytes (NHEK)

### Testing Procedure

The anti-inflammatory activity of a sample is tested against UVB induced NHEK cells in 6-well culture plates. First, NHEK cells are seeded into 6-well plates at a density of 100,000 cells/well. After 24 hours, the cells are washed with warm PBS once and induced by UVB (40 mJ/cm₃). Then, PBS is removed and replaced with fresh medium containing samples or Vitamin D (4 μM). The cells are incubated at 37° C., 5% CO₂ for another 24 hours. Cell culture supernatants are collected and stored at −80° C. before use. Sample preparation: Add 1 ml of B into 49 ml of A to make the sample to be tested. The sample was diluted by 2, 4, 8, 16, 32 and 64 times using NHEK culture medium.

### TNF-α Measurement

a. Removable 8-well strips are labeled for the experiment. The micro-well strips are washed twice with approximately 400 μl wash buffer per well with thorough aspiration of micro-well contents between washes.
b. 100 μl of each standard are added into standard wells. 50 μl sample diluent and 50 μl sample are added into appropriate wells. Then 50 μl Biotin-conjugate is added to all wells.
c. The well is covered and incubated for 2 hours at room temperature with gentle shaking.
d. The solution is discarded and washed 4 times with 1× Wash Solution. Wash by filling each well with Wash Buffer (400 μl) using a multi-channel Pipette. Complete removal of liquid at each step is essential to good performance. After the last wash, remove any remaining Wash Buffer by aspirating or decanting. Invert the plate and blot it against clean paper towels.
e. 100 μl of prepared Streptavidin solution is added to each well. Incubate for 1 hour at room temperature with gentle shaking.
f. Solution is discarded. Repeat the wash as in step d.
g. 100 μl of TMB Substrate Solution is added to each well. Incubate for 30 minutes at room temperature in the dark.
h. 100 μl of Stop Solution is added to each well. Read at 450 nm immediately.

### Analysis Result

Keratinocytes are the major target of UVB and play a central role in inflammatory and immune modulatory changes, through the UV-induced release of pro-inflammatory cytokines (IL-1, IL-6, IL-8, IL-10, GM-CSF and TNF-α), cyclooxygenase products (PGE2), and matrix degrading enzymes as metalloproteinase (MMP). The production of one exemplarily inflammatory cytokine, TNF-α, is measured after UVB at 40 mJ/cm² of NHEK cells. As expected, the exposure to UVB induces an increase of the release of TNF-α (1.6 μg/ml versus 485 μg/ml). The sample shows strongest inhibition on TNF-α production at dilution times of 2 and 4, which is comparable to that of Vitamin D; see FIG. 9.

### Conclusion

In this experiment conditioned medium of the invention reduces the expression/production of a key inflammatory cytokine (TNF-α) by 30×, comparable to Vitamin D.

### Example 11: Conditioned Medium Derived from Cultivation of RD-MLSCs is Effective in Improved Hair Growth in Both Male and Female Patients with Androgenic Alopecia (AGA)

### Participants

Study participants (female n=5, male n=5) with mean age of 34.1±14.2 years were recruited and enrolled in one site located in Vienna, Austria. Patients with manifestation and subjective sensation of hair thinning/hair loss were primarily recruited over social media and study recruitment platforms.

An on-site screening was performed to identify eligible subjects according to Ex- and Inclusion criteria, presented in Table 4, the latter including the Hamilton-Norwood Scale and the Ludwig scale for male and female hair loss patients, respectively. All subjects consented to the use of their demographic as well as study-outcome data for scientific and marketing purposes.

TABLE 4
Inclusion and exclusion criteria

| Inclusion Criteria | Exclusion Criteria |
|---|---|
| Male or female subjects (ratio 1:1) | Patients, who do not return for follow-up |
| Manifestation of hair thinning/hair loss. | review as scheduled. |
| Individual sensation of having less hair, | Patients, who display any adverse |
| suffering from hair loss and thinning hair | reaction to the conditioned medium. |
| Age 18-60 years inclusive | Known Thyroid disfunction |
| | Known deficiencies of vitamins and iron |
| | Smokers |
| | Pregnancy and breastfeeding women |

### Treatment Protocol

Each of the 10 patients disinfected their scalp with 0.5% Chlorhexidine prior to application. The treatment areas were subjectively chosen according to the patient's area of interest and varied slightly from patient to patient. 2.5 ml of a conditioned medium composition of the invention (containing 80% conditioned medium, 20% aqueous hyaluronic acid) was applied onto the treatment area. Afterwards, the respective zone was treated with micro-needling using a 0.5 mm needle length derma stamp, including 3 passes in alternating directions and an additional 2.5 ml conditioned medium composition was applied and gently rubbed into the scalp. The patients were advised not to wash their hair following the treatment for at least 24 hours and optimally 48 hours.

This treatment cycle was repeated weekly for 12 weeks in total. The patients continued with this weekly treatment at home, they were given a derma stamp and product for each week and returned to the clinic at weeks 4, 6, 7, 10 and 12 for on-site assessments. The assessments included subject reported outcomes such as psychological questionnaires, as well as objective measurement of hair growth using the HairMetrix® software by Canfield Scientific.

### Measurements

Phototrichograms for the assessment of hair changes during the treatment period were documented for all patients by 2 trained evaluators by means of the HairMetrix® D200-evo System, using the HairMetrix® software by Canfield Scientific Inc (4 Wood Hollow Road, Parsippany, NJ 07054, USA). Canfield's non-invasive hair consultation tool provides results immediately during the consultation without cutting the hair. HairMetrix® ensures objective data, and the photographs can be taken by study personnel, no extra laboratory assessment is needed.

### Standardized Questionnaires

For the assessment of the patients' quality of life and subjective treatment experience standardized questionnaires have been used (questionnaires not shown).

### Results

10 patients, mean age of 34.1±14.2 y, 50% female, as presented in Table 5, applied the conditioned according to the treatment protocol, as described in the method section. Notably, male patients with Norwood type 4, 5 and 6 hair loss (meaning severe hair loss) participated in the study.

TABLE 5
Basic demographics of patients with hair loss (n = 10) applying
conditioned medium for 12 weeks on their capillitium according
to the treatment protocol, as presented in the method section.

| | | | Norwood-Hamilton/Ludwig |
|---|---|---|---|
| Patient | Sex | Age (years) | classification stage for hair loss |
| 1 | W | 23 | 0-1 |
| 2 | W | 31 | 1-2 |
| 3 | M | 24 | 1 |
| 4 | W | 57 | 1 |
| 5 | W | 19 | 1 |
| 6 | M | 52 | 5 |
| 7 | W | 51 | 1 |
| 8 | M | 23 | 6 |
| 9 | M | 25 | 3-4 |
| 10 | M | 35 | 6 |

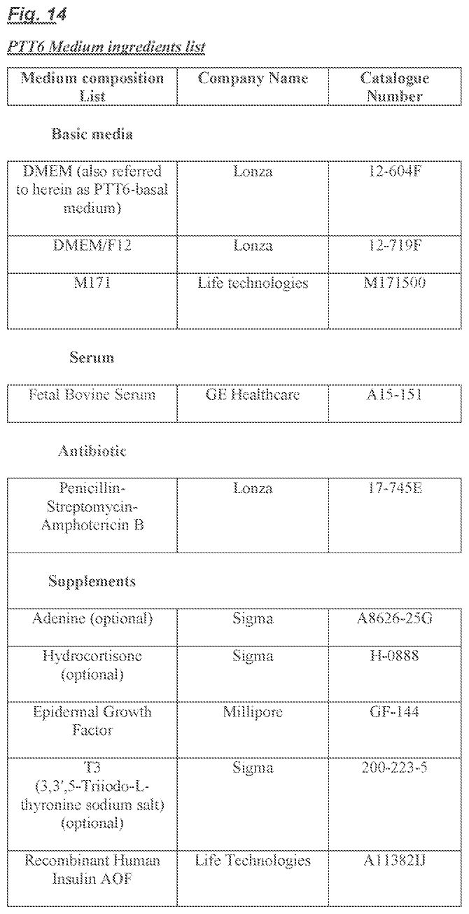

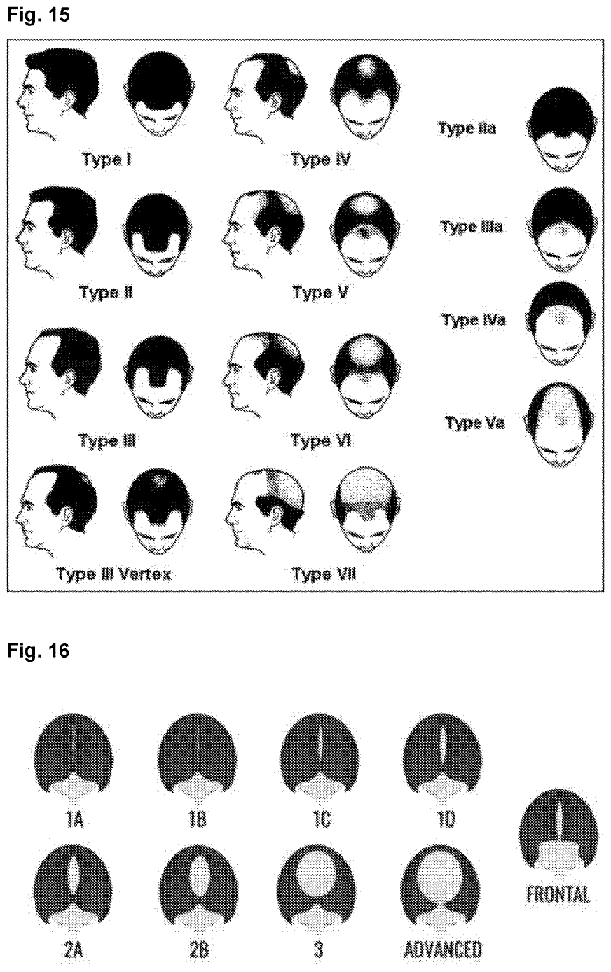

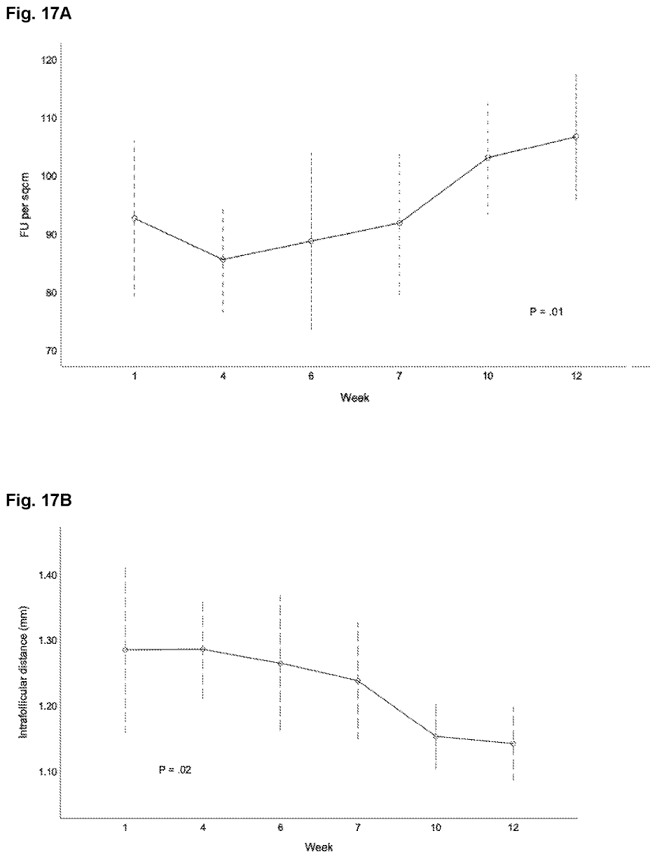

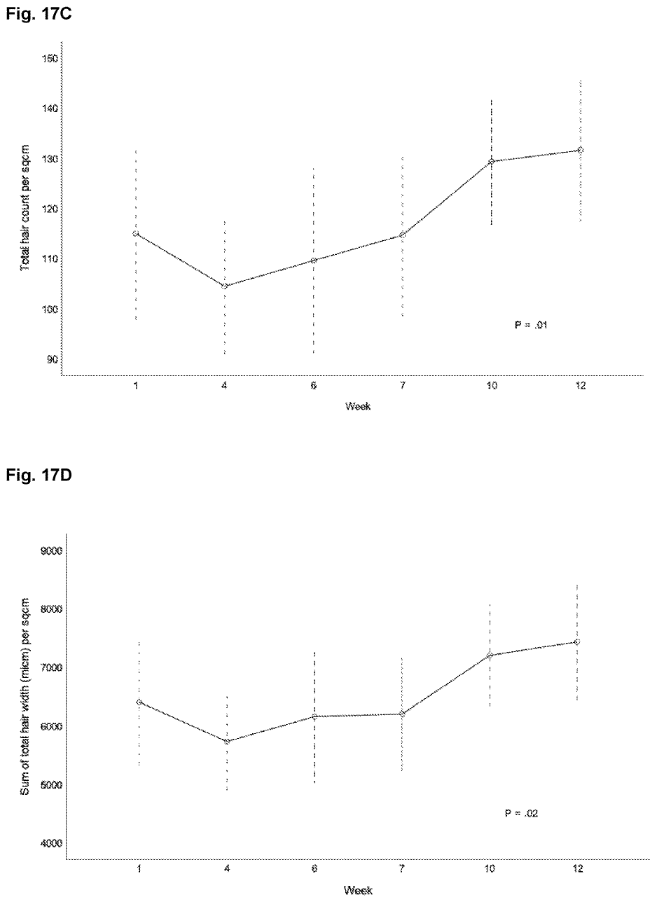

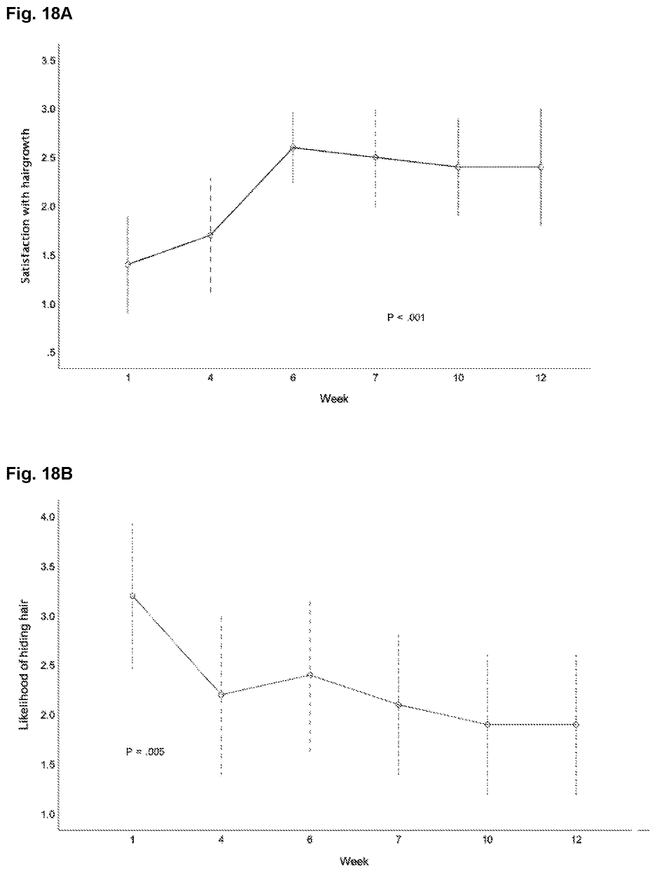

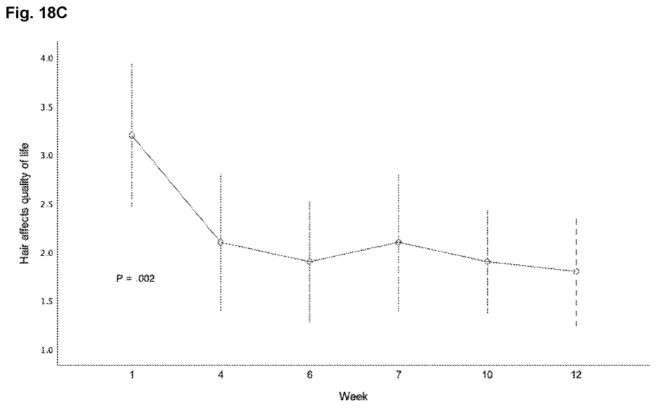

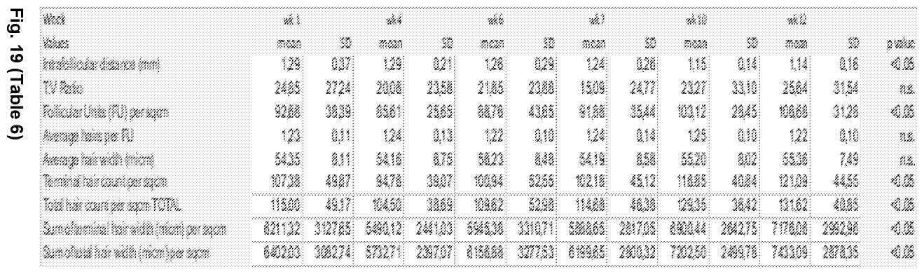

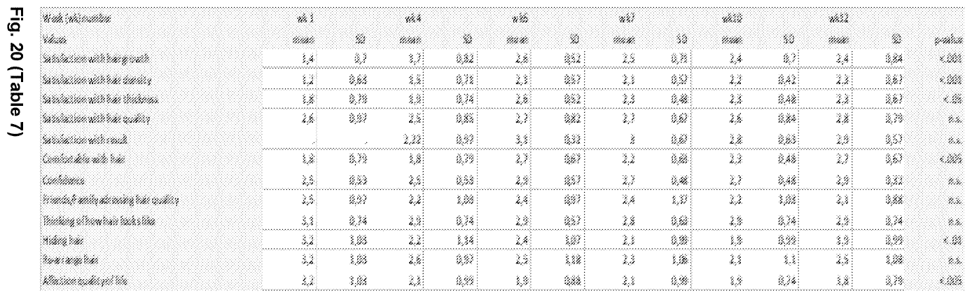

After 12 weeks of treatment, a significant amplification of the follicular units per square centimeter (sqcm) (P<. 05) with an associated decreased intra-follicular distance (P<. 05) was detected, as illustrated in FIG. 17A and FIG. 17B. Likewise, the total hair count per sqcm increased (P<. 05) and thus, the sum of hair width per sqcm (P<. 05), as shown in Table 6 and FIG. 17C and FIG. 17D. Notably, there was neither change in the number of hairs per FU nor in the average hair width. No adverse effects were reported. On the patient's subjective basis, the standardized questionnaires demonstrated a significant improvement of hair growth, less need to hide hair and an increased quality of life associated with hair loss, as presented in Table 7 and FIG. 18A to 18C. Nine out of ten patients said they would recommend the conditioned medium product, showing that the conditioned medium is effective to also treat severe hair loss, for example, of Norwood type 4, 5 and 6.

### Example 12: Encapsulation of Conditioned Medium Derived from Cultivation of RD-MLSCs in Liposomal Formulation and Use of this Formulation for Improving Hair Growth in Patients with Androgenic Alopecia (AGA)

Pro-Lipo™ Neo, a mixture of phospholipids commercially available from Lucas Meyer Cosmetics, was used to encapsulate conditioned medium derived from cultivation of RD-MLSCs to form bilamellar liposomes that are suitable for topical administration. This liposomal formulation was then used for treatment of patients suffering from AGA. In this study, conditioned medium was used together with other compounds that are reported to prevents and halts hair loss and/or at the same time stimulate hair growth. Capixyl™, a bio-mimetic peptide (Acetyl tetrapeptide-3), available from Lucas Meyer Cosmetics, ribose, menthol and caffeine were used as compounds for which hair growth stimulating effects have been reported. The encapsulated hair serum produced in this experiment had the following composition:

### Encapsulated Hair Serum

a. conditioned medium: 50% (v/v)
b. Capixyl™ (acetyl tetrapeptide-3): 2% (indicative, v/v))
c. Riboxyl (Ribose): 0.5% (indicative, v/v)
d. Pro-Lipo Neo (Liposomal Mixture)—Delivery system: 20% (v/v)
e. Menthol: 0.1% (indicative, v (v))
f. Caffeine: 2% (indicative, v/v))
   with the balance being excipient

The encapsulated hair serum was formulated as follows:

### Step 1: Liposomal Encapsulation of Active Ingredients

1. Thaw the conditioned medium (CM).
2. Combine CM, Capixyl™, Riboxyl, caffeine and PLN by stirring under low shear.
3. Stir for 30 minutes until homogenous.
   Step 2: Incorporation of Menthol into Serum Base
1. Dissolve menthol in the excipient base
2. Turn off the heating to cool down the excipient base.
   Step 3: Incorporation of Liposomal-Encapsulated Actives into Serum Base
1. Combine liposomal-encapsulated active ingredients, excipient base and preservatives by stirring under low shear until homogenous.
2. Ensure that the pH of the final product is 7.
   Since the liposomal formulation should improve cutaneous absorption of the conditioned medium after topical administration of the scalp, the hair serum so produced was topically applied on the scalp of patients suffering from AGA without micro needling and use of a derma stamp. Over the course of the treatment with this liposomal formulation of conditioned medium, significant improvement of hair growth was observed (data not shown), showing the effectiveness of this formulation for inducing and promoting hair growth.

The invention is also characterized by the following items.
1. A method of inducing, stimulating and/or promoting hair growth and/or hair regeneration, wherein the method comprises treating the hair of a subject with conditioned medium derived from cultivation of mesenchymal stem cells of the umbilical cord.
2. A method of alleviating and/or reducing hair loss and/or hair thinning, wherein the method comprises treating the hair of a subject with conditioned medium derived from cultivation of mesenchymal stem cells of the umbilical cord.
3. The method of item 1 or 2, wherein the conditioned medium is obtained or obtainable by cultivating the mesenchymal stem cells of the umbilical cord in a culture medium comprising DMEM (Dulbecco's modified eagle medium), F12 (Ham's F12 Medium), M171 (Medium 171) and FBS (Fetal Bovine Serum).
4. The method of item 3, wherein the culture medium comprises DMEM in a final concentration of about 55 to 65% (v/v), F12 in a final concentration of about 5 to 15% (v/v), M171 in a final concentration of about 15 to 30% (v/v) and FBS in a final concentration of about 1 to 8% (v/v).
5. The method of item 3 or 4, wherein the culture medium comprises DMEM in a final concentration of about 57.5 to 62.5% (v/v), F12 in a final concentration of about 7.5 to 12.5% (v/v), M171 in a final concentration of about 17.5 to 25.0% (v/v) and FBS in a final concentration of about 1.75 to 3.5% (v/v).
6. The method of any of items 3-5, wherein the culture medium comprises DMEM in a final concentration of about 61.8% (v/v), F12 in a final concentration of about 11.8% (v/v), M171 in a final concentration of about 23.6% (v/v) and FBS in a final concentration of about 2.5% (v/v).
7. The method of any of items 3-6, wherein the culture medium further comprises
(i) Epidermal Growth Factor (EGF) in a final concentration of 1 ng/ml to 20 ng/ml, or
(ii) wherein the culture medium comprises EGF in a final concentration of 10 ng/ml, or
(iii) wherein the culture medium comprises Insulin in a final concentration of 1 g/ml to 10 μg/ml, or
(iv) wherein the culture medium comprises Insulin in a final concentration of 5 μg/ml, or
(v) wherein the culture medium further comprises at least one of the following supplements: adenine, hydrocortisone, and 3,3′,5-Triiodo-L-thyronine sodium salt (T3), or
(vi) wherein the culture medium comprises all three of adenine, hydrocortisone, and 3,3′,5-Triiodo-L-thyronine sodium salt (T3), wherein optionally the culture medium comprises adenine in a final concentration of 0.01 to 0.1 μg/ml adenine, hydrocortisone in a final concentration of 0.1 to 10 μg/ml hydrocortisone and/or 3,3′,5-Triiodo-L-thyronine sodium salt (T3) in a final concentration of 0.5 to 5 ng/ml.
8. The method of any of items 1-7, wherein the mesenchymal stem cells of the umbilical cord are human or red deer mesenchymal stem cells of the umbilical cord.
9. The method of any of items 1-8, wherein the conditioned medium is derived from cultivation of a mesenchymal stem cell population of the amniotic membrane of the umbilical cord, from cultivation of a mesenchymal stem cell population of Wharton's jelly of the umbilical cord (WJ), from cultivation of a perivascular (PV) mesenchymal stems cell population, or from cultivation of a mixed mesenchymal stem cell population of the umbilical cord (MC).
10. The method of any of items 1-9, wherein the conditioned medium is applied topically to the skin, particularly to the scalp, of a subject.
11. The method of any of items 1-10, wherein the conditioned medium is applied to the hair line of a subject.
12. The method of any of items 1-11, wherein the conditioned medium is administered about once, twice or three times a week for a period of time of three weeks, of four weeks, or five weeks or of six weeks, or of seven weeks, or of eight weeks or of ten weeks or more weeks.
13. The method of any of items 1-12, wherein the subject is suffering from hair loss/hair thinning, particularly inflammatory-induced hair loss/hair thinning such as chronic inflammatory-induced hair loss.
14. The method of any of items 1-13, wherein the subject is suffering from a disease or condition associated with hair loss/hair thinning, particularly alopecia such as androgenic alopecia, alopecia areata, alopecia totalis, alopecia universalis or COVID-19 induced alopecia.
15. A method of producing a conditioned medium, the method comprising
(a) cultivating mesenchymal stem cells of the umbilical cord in a culture medium comprising DMEM (Dulbecco's modified eagle medium), F12 (Ham's F12 Medium), M171 (Medium 171) and FBS (Fetal Bovine Serum);
(b) removing the mesenchymal stem cells of the umbilical cord from the culture medium;
(c) optionally cultivating the mesenchymal stem cells of the umbilical cord in a further culture medium, optionally wherein the cell culture medium comprises a water-soluble antioxidant, wherein the conditioned medium is obtained by collecting the cell culture medium.
16. The method of item 15, wherein the cell culture medium does not comprise a growth factor and/or serum and/or wherein the cells are cultivated at a concentration of about 1 million cells per 1 ml.
17. The method of item 16, wherein the mesenchymal stem cells of the umbilical cord are red deer mesenchymal stem cells of the umbilical cord.
18. The method of item 16 or 17, wherein the conditioned medium derived from mesenchymal stem cells of the umbilical cord is used for inducing, stimulating and/or promoting hair growth and/or hair regeneration; and/or for alleviating and/or reducing hair loss and/or hair thinning of a subject.
19. The method of any of items 16-18, wherein the conditioned medium is diluted in control medium.
20. The method of any of items 1-14, wherein the conditioned medium is obtained or obtainable by the method according to any of items 15-19.
21. A conditioned medium derived from mesenchymal stem cells of the umbilical cord obtained or obtainable by the method according to any of items 15-19.
22. A composition comprising the conditioned medium of item 21.
23. Use of conditioned medium derived from cultivation of mesenchymal stem cells of the umbilical cord for inducing, stimulating and/or promoting hair growth and/or hair regeneration; and/or for alleviating and/or reducing hair loss and/or hair thinning of a subject.
24. The use of item 23, wherein the conditioned medium derived from cultivation of mesenchymal stem cells of the umbilical cord is obtained or obtainable by the method according to any of items 15-19.
25. The use of conditioned medium derived from cultivation of mesenchymal stem cells of the umbilical cord for the manufacture of a medicament for inducing, stimulating and/or promoting hair growth and/or hair regeneration; and/or for alleviating and/or reducing hair loss and/or hair thinning of a subject.
26. The use of conditioned medium derived of item 25, wherein the use comprises treating the hair of a subject with conditioned medium derived from cultivation of mesenchymal stem cells of the umbilical cord.
27. The use of item 25 or 26, wherein the conditioned medium is obtained or obtainable by cultivating the mesenchymal stem cells of the umbilical cord in a culture medium comprising DMEM (Dulbecco's modified eagle medium), F12 (Ham's F12 Medium), M171 (Medium 171) and FBS (Fetal Bovine Serum).
28. The use of item 27, wherein the culture medium comprises DMEM in a final concentration of about 55 to 65% (v/v), F12 in a final concentration of about 5 to 15% (v/v), M171 in a final concentration of about 15 to 30% (v/v) and FBS in a final concentration of about 1 to 8% (v/v).
29. The use of item 27 or 28, wherein the culture medium comprises DMEM in a final concentration of about 57.5 to 62.5% (v/v), F12 in a final concentration of about 7.5 to 12.5% (v/v), M171 in a final concentration of about 17.5 to 25.0% (v/v) and FBS in a final concentration of about 1.75 to 3.5% (v/v).
30. The use of any of items 26-28, wherein the culture medium comprises DMEM in a final concentration of about 61.8% (v/v), F12 in a final concentration of about 11.8% (v/v), M171 in a final concentration of about 23.6% (v/v) and FBS in a final concentration of about 2.5% (v/v).
31. The use of any of items 26-30, wherein the culture medium further comprises
(i) Epidermal Growth Factor (EGF) in a final concentration of 1 ng/ml to 20 ng/ml, or
(ii) wherein the culture medium comprises EGF in a final concentration of 10 ng/ml, or
(iii) wherein the culture medium comprises Insulin in a final concentration of 1 μg/ml to 10 μg/ml, or
(iv) wherein the culture medium comprises Insulin in a final concentration of 5 μg/ml, or
(v) wherein the culture medium further comprises at least one of the following supplements: adenine, hydrocortisone, and 3,3′,5-Triiodo-L-thyronine sodium salt (T3), or
(vi) wherein the culture medium comprises all three of adenine, hydrocortisone, and 3,3′,5-Triiodo-L-thyronine sodium salt (T3), wherein optionally the culture medium comprises adenine in a final concentration of 0.01 to 0.1 μg/ml adenine, hydrocortisone in a final concentration of 0.1 to 10 μg/ml hydrocortisone and/or 3,3′,5-Triiodo-L-thyronine sodium salt (T3) in a final concentration of 0.5 to 5 ng/ml.
32. The use of any of items 25-31, wherein the mesenchymal stem cells of the umbilical cord are human or red deer mesenchymal stem cells of the umbilical cord.
33. The use of any of items 25-32, wherein the conditioned medium is derived from cultivation of a mesenchymal stem cell population of the amniotic membrane of the umbilical cord, from cultivation of a mesenchymal stem population of the Wharton's jelly of the umbilical cord (WJ), from cultivation of a perivascular (PV) mesenchymal stems cell population, or from cultivation of a mixed mesenchymal stem cell population of the umbilical cord (MC).
34. The use of any of items 25-33, wherein the use comprises topically applying the conditioned medium i to the skin, particularly to the scalp, of a subject.
35. The use of any of items 25-34, wherein the use comprises applying the conditioned medium to the hair line of a subject.
36. The use of any of items 25-35, wherein the conditioned medium is administered about once, twice or three times a week for a period of time of three weeks, of four weeks, or five weeks or of six weeks, or of seven weeks, or of eight weeks or of ten weeks or more weeks.
37. The use of any of items 25-36, wherein the subject is suffering from hair loss/hair thinning, particularly inflammatory-induced hair loss/hair thinning such as chronic inflammatory-induced hair loss.
38. The use of any of items 25-37, wherein the subject is suffering from a disease or condition associated with hair loss/hair thinning, particularly alopecia such as androgenic alopecia, alopecia areata, alopecia totalis, alopecia universalis or COVID-19 induced alopecia.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

What is claimed is:

1. A non-therapeutic method of inducing, stimulating and/or promoting hair growth and/or hair regeneration, wherein the method comprises treating the hair of a subject with conditioned medium derived from cultivation of a mesenchymal stem cell population of the umbilical cord, wherein the mesenchymal stem cell population is a mesenchymal stem cell population of the amniotic membrane of the umbilical cord or a mesenchymal stem population of the Wharton's jelly of the umbilical cord,
wherein the conditioned medium is obtained by
a) cultivating the mesenchymal stem cells of the umbilical cord in a culture medium comprising DMEM (Dulbecco's modified eagle medium), F12 (Ham's F12 Medium), M171 (Medium 171) and FBS (Fetal Bovine Serum),
wherein the culture medium comprises DMEM in a final concentration of 55 to 65% (v/v), F12 in a final concentration of 5 to 15% (v/v), M171 in a final concentration of 15 to 30% (v/v) and FBS in a final concentration of 1 to 8% (v/v).
b) removing the mesenchymal stem cells of the umbilical cord from the culture medium; and cultivating the mesenchymal stem cells of the umbilical cord in a further culture medium, optionally wherein the further cell culture medium comprises a water-soluble antioxidant,
wherein the further medium is a basal medium suitable for cultivation of mesenchymal stem cells, and
c) collecting the further cell culture medium.

2. A non-therapeutic method of alleviating and/or reducing hair loss and/or hair thinning, wherein the method comprises treating the hair of a subject with conditioned medium derived from cultivation of a mesenchymal stem cell population of the umbilical cord, wherein the mesenchymal stem cell population is a mesenchymal stem cell population of the amniotic membrane of the umbilical cord or a mesenchymal stem population of the Wharton's jelly of the umbilical cord,
wherein the conditioned medium is obtained by
(a) cultivating the mesenchymal stem cells of the umbilical cord in a culture medium comprising DMEM (Dulbecco's modified eagle medium), F12 (Ham's F12 Medium), M171 (Medium 171) and FBS (Fetal Bovine Serum),
wherein the culture medium comprises DMEM in a final concentration of 55 to 65% (v/v), F12 in a final concentration of 5 to 15% (v/v), M171 in a final concentration of 15 to 30% (v/v) and FBS in a final concentration of 1 to 8% (v/v).
(b) removing the mesenchymal stem cells of the umbilical cord from the culture medium; and cultivating the mesenchymal stem cells of the umbilical cord in a further culture medium, optionally wherein the further cell culture medium comprises a water-soluble antioxidant,
wherein the further medium is a basal medium suitable for cultivation of mesenchymal stem cells, and
(c) collecting the further cell culture medium.

3. The method of claim 1 or 2, wherein the culture medium comprises DMEM in a final concentration of 57.5 to 62.5% (v/v), F12 in a final concentration of 7.5 to 12.5% (v/v), M171 in a final concentration of 17.5 to 25.0% (v/v) and FBS in a final concentration of 1.75 to 3.5% (v/v).

4. The method of claim 3, wherein the culture medium comprises DMEM in a final concentration of 61.8% (v/v), F12 in a final concentration of 11.8% (v/v), M171 in a final concentration of 23.6% (v/v) and FBS in a final concentration of 2.5% (v/v).

5. The method of any of claims 3-4, wherein the culture medium further comprises
(i) Epidermal Growth Factor (EGF) in a final concentration of 1 ng/ml to 20 ng/ml, or
(ii) wherein the culture medium comprises EGF in a final concentration of 10 ng/ml, or
(iii) wherein the culture medium comprises Insulin in a final concentration of 1 μg/ml to 10 μg/ml, or
(iv) wherein the culture medium comprises Insulin in a final concentration of 5 μg/ml, or
(v) wherein the culture medium further comprises at least one of the following supplements: adenine, hydrocortisone, and 3,3′,5-Triiodo-L-thyronine sodium salt (T3), or
(vi) wherein the culture medium comprises all three of adenine, hydrocortisone, and 3,3′,5-Triiodo-L-thyronine sodium salt (T3),
wherein optionally the culture medium comprises adenine in a final concentration of 0.01 to 0.1 μg/ml adenine, hydrocortisone in a final concentration of 0.1 to 10 μg/ml hydrocortisone and/or 3,3′,5-Triiodo-L-thyronine sodium salt (T3) in a final concentration of 0.5 to 5 ng/ml.

6. The method of any of claims 1-5, wherein the further cell culture medium does not comprise a growth factor and/or is a serum-free medium and/or wherein the cells are cultivated at a concentration of 1 million cells per 1 ml.

7. The method of claim 6, wherein the further medium is a serum free basal medium.

8. The method of claim 7, wherein the basal medium is selected from the group consisting of Dulbecco's Modified Eagle Medium (DMEM), DMEM-F12, RPMI media, EpiLlfe medium, and Medium 171.

9. The method of any of claims 6 to 8, wherein the conditioned medium is diluted in a carrier medium, wherein the final concentration of the conditioned medium is preferably from 10% to 90% (v/v) of the total volume of the composition that contains the conditioned medium, including a final concentration of 20%, of 25%, of 30%, of 35%, of 40%, of 45%, of 50%, of 55%, of 60%, of 65%, of 70%, of 75% or of 80%.

10. The method of any of claims 1-9, wherein the mesenchymal stem cell population of the umbilical cord is a human or a red deer mesenchymal stem cell population of the umbilical cord.

11. The method of any of claims 1-10, wherein treating comprises topically applying the conditioned medium to the skin, particularly to the scalp, of a subject or injecting the conditioned medium into the skin, particularly the scalp of a subject.

12. The method of any of claims 1-11, wherein treating comprises applying the conditioned medium to the hair line of a subject.

13. The method of any of claims 1-12, wherein the conditioned medium is administered once, twice or three times a week for a period of time of three weeks, of four weeks, or five weeks or of six weeks, or of seven weeks, or of eight weeks or of ten weeks or more weeks.

14. The method of any of claims 1-13, wherein the subject is suffering from hair loss/hair thinning, particularly inflammatory-induced hair loss/hair thinning, hair loss/hair thinning due to a viral or bacterial infection, hair loss due to drug treatment or hormone-related hair loss.

15. The method of claim 14, wherein the inflammatory-induced hair loss/hair thinning is chronic inflammatory-induced hair loss.

16. The method of claim 14, wherein the hormone-related hair loss/hair thinning is menopausal related hair loss or postpartum related hair loss.

17. The method of claim 14, wherein the hair loss due to drug treatment is hair loss due to cancer chemotherapy.

18. The method of any of claims 1-17, wherein the subject is suffering from a disease or condition associated with hair loss/hair thinning, particularly telogen effluvium, alopecia such as androgenic alopecia, alopecia areata, alopecia universalis or COVID-19 induced alopecia.

19. The method of claim 18, wherein the alopecia areata is alopecia areata monolocularis, alopecia areata multilocularis, ophiasis, alopecia totalis or alopecia universalis.

20. The method of any of claims 1 to 19, wherein the hair loss is male hair loss or female hair loss.

21. The method of claim 20, wherein the subject is a male and wherein the male has been diagnosed with hair loss of Type I, Type II, Type III, Type IV, Type V or Type VI hair loss according to the Norwood classification.

22. The method of claim 20, wherein the subject is a female and wherein the female has been diagnosed with hair loss of Type I (including Type Ia, Type Ib, Type Ic and Type Id), Type II (Type IIa and Type IIb) or Type III hair loss according to the Ludwig classification.

23. A method of producing a conditioned medium, the method comprising
(a) cultivating mesenchymal stem cells of the umbilical cord, wherein the mesenchymal stem cells of the umbilical cord are derived from a mesenchymal stem cell population of the amniotic membrane of the umbilical cord or from a mesenchymal stem population of the Wharton's jelly of the umbilical cord, in a culture medium comprising DMEM (Dulbecco's modified eagle medium), F12 (Ham's F12 Medium), M171 (Medium 171) and FBS (Fetal Bovine Serum), wherein the culture medium comprises DMEM in a final concentration of 55 to 65% (v/v), F12 in a final concentration of 5 to 15% (v/v), M171 in a final concentration of 15 to 30% (v/v) and FBS in a final concentration of 1 to 8% (v/v);
(b) removing the mesenchymal stem cells of the umbilical cord from the culture medium; and
(c) cultivating the mesenchymal stem cells of the umbilical cord in a further culture medium, wherein the further medium is a basal medium suitable for cultivation of mesenchymal stem cells, and wherein the further cell culture medium comprises a water-soluble antioxidant,
wherein the conditioned medium is obtained by collecting the further cell culture medium.

24. The method of claim 23, wherein the further cell culture medium does not comprise a growth factor and/or is a serum-free medium and/or wherein the cells are cultivated at a concentration of 1 million cells per 1 ml.

25. The method of claim 24, wherein the mesenchymal stem cells of the umbilical cord are human or red deer mesenchymal stem cells of the umbilical cord.

26. The method of any of claim 24 or 25, wherein the further medium is a serum free medium.

27. The method of claim 26, wherein the basal medium is selected from the group consisting of Dulbecco's Modified Eagle Medium (DMEM), DMEM-F12, RPMI media, EpiLlfe medium, and Medium 171. 28 The method of any of claims 23 to 27, wherein the culture medium comprises DMEM in a final concentration of 57.5 to 62.5% (v/v), F12 in a final concentration of 7.5 to 12.5% (v/v), M171 in a final concentration of 17.5 to 25.0% (v/v) and FBS in a final concentration of 1.75 to 3.5% (v/v). 29 The method of claim 28, wherein the culture medium comprises DMEM in a final concentration of 61.8% (v/v), F12 in a final concentration of 11.8% (v/v), M171 in a final concentration of 23.6% (v/v) and FBS in a final concentration of 2.5% (v/v).

30. The method of any of claims 23 to 29, wherein the conditioned medium is diluted in a carrier medium, wherein the final concentration of the conditioned medium is preferably from 10% to 90% (v/v) of the total volume of the composition that contains the conditioned medium, including a final concentration of 20%, of 25%, of 30%, of 35%, of 40%, of about 45%, of 50%, of 55%, of 60%, of 65%, of 70%, of 75% or of 80%.

31. The method of claim 30, wherein the carrier medium is a physiological buffer, water, an aqueous solution of a extracellular matrix component, or a basal medium.

32. The method of claim 31, wherein the aqueous solution of an extracellular matrix component is an aqueous solution of hyaluronic acid, an aqueous solution of collagen or an aqueous solution of fibronectin.

33. The method of any of claims 23 to 32, wherein the conditioned medium is encapsulated into liposomes.

34. The method of claim 33, wherein the liposomes are made from a phospholipid mixture.

35. A conditioned medium derived from mesenchymal stem cells of the umbilical cord obtained or obtainable by the method according to any of claims 23 to 34.

36. A composition comprising the conditioned medium of claim 35.

37. The use of conditioned medium derived from cultivation of a mesenchymal stem cell population of the umbilical cord for the manufacture of a medicament for inducing, stimulating and/or promoting hair growth and/or hair regeneration; and/or for alleviating and/or reducing hair loss and/or hair thinning of a subject,
wherein the mesenchymal stem cell population is a mesenchymal stem cell population of the amniotic membrane of the umbilical cord or a mesenchymal stem population of the Wharton's jelly of the umbilical cord,
wherein the conditioned medium is obtained by
a) cultivating the mesenchymal stem cells of the umbilical cord in a culture medium comprising DMEM (Dulbecco's modified eagle medium), F12 (Ham's F12 Medium), M171 (Medium 171) and FBS (Fetal Bovine Serum),
wherein the culture medium comprises DMEM in a final concentration of 55 to 65% (v/v), F12 in a final concentration of 5 to 15% (v/v), M171 in a final concentration of 15 to 30% (v/v) and FBS in a final concentration of 1 to 8% (v/v).
b) removing the mesenchymal stem cells of the umbilical cord from the culture medium; and cultivating the mesenchymal stem cells of the umbilical cord in a further culture medium, optionally wherein the further cell culture medium comprises a water-soluble antioxidant,
wherein the further medium is a basal medium suitable for cultivation of mesenchymal stem cells, and
c) collecting the further cell culture medium.

38. The use of claim 37, wherein the use comprises treating the hair of a subject with conditioned medium derived from cultivation of mesenchymal stem cells of the umbilical cord.

39. The use of claim 37 or 38, wherein the culture medium comprises DMEM in a final concentration of 57.5 to 62.5% (v/v), F12 in a final concentration of 7.5 to 12.5% (v/v), M171 in a final concentration of 17.5 to 25.0% (v/v) and FBS in a final concentration of 1.75 to 3.5% (v/v).

40. The use of any of claims 37 to 39, wherein the culture medium comprises DMEM in a final concentration of 61.8% (v/v), F12 in a final concentration of 11.8% (v/v), M171 in a final concentration of 23.6% (v/v) and FBS in a final concentration of 2.5% (v/v).

41. The use of any of claims 37 to 40, wherein the culture medium further comprises
(i) Epidermal Growth Factor (EGF) in a final concentration of 1 ng/ml to 20 ng/ml, or
(ii) wherein the culture medium comprises EGF in a final concentration of 10 ng/ml, or
(iii) wherein the culture medium comprises Insulin in a final concentration of 1 μg/ml to 10 μg/ml, or
(iv) wherein the culture medium comprises Insulin in a final concentration of 5 μg/ml, or
(v) wherein the culture medium further comprises at least one of the following supplements: adenine, hydrocortisone, and 3,3′,5-Triiodo-L-thyronine sodium salt (T3), or
(vi) wherein the culture medium comprises all three of adenine, hydrocortisone, and 3,3′,5-Triiodo-L-thyronine sodium salt (T3),
wherein optionally the culture medium comprises adenine in a final concentration of 0.01 to 0.1 μg/ml adenine, hydrocortisone in a final concentration of 0.1 to 10 μg/ml hydrocortisone and/or 3,3′,5-Triiodo-L-thyronine sodium salt (T3) in a final concentration of 0.5 to 5 ng/ml.

42. The use of any of claims 37 to 41, wherein the mesenchymal stem cells of the umbilical cord are human or red deer mesenchymal stem cells of the umbilical cord.

43. The use of any of claims 37 to 42, wherein the use comprises topically applying the conditioned medium to the skin, particularly to the scalp, of a subject.

44. The use of any of claims 37 to 43, wherein the use comprises applying the conditioned medium to the hair line of a subject.

45. The use of any of claims 37 to 44, wherein the conditioned medium is administered once, twice or three times a week for a period of time of three weeks, of four weeks, or five weeks or of six weeks, or of seven weeks, or of eight weeks or of ten weeks or more weeks.

46. The use of any of claims 37 to 45, wherein the subject is suffering from hair loss/hair thinning, particularly inflammatory-induced hair loss/hair thinning, hair loss/hair thinning due to a viral or bacterial infection, hair loss due to drug treatment or hormone-related hair loss.

47. The use of claim 46, wherein the inflammatory-induced hair loss/hair thinning is chronic inflammatory-induced hair loss.

48. The use of claim 47, wherein the hormone-related hair loss/hair thinning is menopausal related hair loss or postpartum related hair loss.

49. The use of claim 46, wherein the hair loss due to drug treatment is hair loss due to cancer chemotherapy.

50. The use of any of claims 37-47, wherein the subject is suffering from a disease or condition associated with hair loss/hair thinning, particularly telogen effluvium, alopecia such as androgenic alopecia, alopecia areata, alopecia universalis or COVID-19 induced alopecia.

51. The use of claim 50, wherein the alopecia areata is alopecia areata monolocularis, alopecia areata multilocularis, ophiasis, alopecia totalis or alopecia universalis.

52. The use of any of claims 37 to 60, wherein the hair loss is male hair loss or female hair loss.

53. The use of claim 52, wherein the subject is a male and wherein the male has been diagnosed from hair loss of Type I, Type II, Type III, Type IV or Type V hair loss according to the Norwood classification.

54. The use of claim 52, wherein the subject is a female and wherein the female has been diagnosed with hair loss of Type I (including Type Ia, Type Ib, Type Ic and Type Id), Type II (Type IIa and Type IIb) or Type III hair loss according to the Ludwig classification.


SEQUENCE LISTING (17 sequences)

SEQ ID NO: 1 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 2 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 3 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 4 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 5 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 6 - protein, 211 aa, Homo sapiens [sequence omitted]

SEQ ID NO: 7 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 8 - protein, 210 aa, Homo sapiens
MGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 9 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 10 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 11 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 12 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 13 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 14 - protein, 259 aa, Homo sapiens
MARIPKTLKFVVVIVAVLLPVLAYSATTARQEEVPQQTVAPQQQRHSFKGEECPAGSHRSEHTGACNPCTEGVDYTNASNNEPSCFPCTVCKSDQKHKSSCTMTRDTVCQCKEGTFRNENSPEMCRKCSRCPSGEVQVSNCTSWDDIQCVEEFGANATVETPAAEETMNTSPGTPAPAAEETMNTSPGTPAPAAEETMTTSPGTPAPAAEETMTTSPGTPAPAAEETMTTSPGTPASSHYLSCTIVGIIVLIVLLIVFV

SEQ ID NO: 15 - protein, 335 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 16 - protein, 531 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVVLSPRYEIVIITVVAAAVIMGTAGLSTYLYNRQRKIKKYRLQQAQKGTPMKPNTQATPP

SEQ ID NO: 17 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK